SEQUENCE LISTING (32 sequences)

SEQ ID NO: 1 - DNA, 19 bp, ARTIFICIAL SEQUENCE
  Primer sequence for qRT-PCR
atggacaaggcgcagatcc

SEQ ID NO: 2 - DNA, 18 bp, ARTIFICIAL SEQUENCE
  Primer sequence for qRT-PCR
ctccgacttgtcccccat

SEQ ID NO: 3 - DNA, 20 bp, ARTIFICIAL SEQUENCE
  Primer sequence for qRT-PCR
atcccctgagggcacactta

SEQ ID NO: 4 - DNA, 21 bp, ARTIFICIAL SEQUENCE
  Primer sequence for qRT-PCR
ggaatggtgatctccgctgac

SEQ ID NO: 5 - DNA, 21 bp, ARTIFICIAL SEQUENCE
  Primer sequence for qRT-PCR
ttcctcaactgctacatcgct

SEQ ID NO: 6 - DNA, 20 bp, ARTIFICIAL SEQUENCE
  Primer sequence for qRT-PCR
cctgtcgctgtactcgttgg

SEQ ID NO: 7 - DNA, 21 bp, ARTIFICIAL SEQUENCE
  Primer sequence for qRT-PCR
cacagtccttcgccagatgag

SEQ ID NO: 8 - DNA, 23 bp, ARTIFICIAL SEQUENCE
  Primer sequence for qRT-PCR
ctacaccttgaagcattaaggct

SEQ ID NO: 9 - DNA, 22 bp, ARTIFICIAL SEQUENCE
  Primer sequence for qRT-PCR
agtttcttccgctctttgacag

SEQ ID NO: 10 - DNA, 22 bp, ARTIFICIAL SEQUENCE
  Primer sequence for qRT-PCR
tgccacctttggttacagtttc

SEQ ID NO: 11 - DNA, 20 bp, ARTIFICIAL SEQUENCE
  Primer sequence for qRT-PCR
gggctagacgtaggctcaca

SEQ ID NO: 12 - DNA, 22 bp, ARTIFICIAL SEQUENCE
  Primer sequence for qRT-PCR
ccaccatttttcattgggacca

SEQ ID NO: 13 - DNA, 21 bp, ARTIFICIAL SEQUENCE
  Primer sequence for qRT-PCR
gttcttcggagagcacctgtt

SEQ ID NO: 14 - DNA, 21 bp, ARTIFICIAL SEQUENCE
  Primer sequence for qRT-PCR
gagagtccggtgtcaatccag

SEQ ID NO: 15 - DNA, 20 bp, ARTIFICIAL SEQUENCE
  Primer sequence for qRT-PCR
ggctgtattcccctccatcg

SEQ ID NO: 16 - DNA, 22 bp, ARTIFICIAL SEQUENCE
  Primer sequence for qRT-PCR
ccagttggtaacaatgccatgt

SEQ ID NO: 17 - DNA, 21 bp, ARTIFICIAL SEQUENCE
  Primer sequence for qRT-PCR
ctacacgcagttgcagtacat

SEQ ID NO: 18 - DNA, 20 bp, ARTIFICIAL SEQUENCE
  Primer sequence for qRT-PCR
cagcaggatctggatctccc

SEQ ID NO: 19 - DNA, 22 bp, ARTIFICIAL SEQUENCE
  Primer sequence for qRT-PCR
tacaccaacctctcgtacatcg

SEQ ID NO: 20 - DNA, 23 bp, ARTIFICIAL SEQUENCE
  Primer sequence for qRT-PCR
catgtctgaagcgcagtaagatt

SEQ ID NO: 21 - DNA, 21 bp, ARTIFICIAL SEQUENCE
  Primer sequence for qRT-PCR
catgtacgttgctatccaggc

SEQ ID NO: 22 - DNA, 21 bp, ARTIFICIAL SEQUENCE
  Primer sequence for qRT-PCR
ctccttaatgtcacgcacgat

SEQ ID NO: 23 - DNA, 25 bp, ARTIFICIAL SEQUENCE
  Primer sequence for ChIP
ggcgaaacccctggaatattcccga

SEQ ID NO: 24 - DNA, 19 bp, ARTIFICIAL SEQUENCE
  Primer sequence for ChIP
agccttgggacaacgggag

SEQ ID NO: 25 - DNA, 18 bp, ARTIFICIAL SEQUENCE
  Primer sequence for ChIP
ctcctttccccgatcctc

SEQ ID NO: 26 - DNA, 18 bp, ARTIFICIAL SEQUENCE
  Primer sequence for ChIP
caacggctgcccaagaag

SEQ ID NO: 27 - DNA, 19 bp, ARTIFICIAL SEQUENCE
  Primer sequence for ChIP
gtcgcgctctcgaattcat

SEQ ID NO: 28 - DNA, 18 bp, ARTIFICIAL SEQUENCE
  Primer sequence for ChIP
cctccccatgcactcctc

SEQ ID NO: 29 - DNA, 22 bp, ARTIFICIAL SEQUENCE
  Primer sequence for ChIP
gtgtagaccttttagccgatgc

SEQ ID NO: 30 - DNA, 21 bp, ARTIFICIAL SEQUENCE
  Primer sequence for ChIP
gtgccagtacagtagcagtgg

SEQ ID NO: 31 - DNA, 20 bp, ARTIFICIAL SEQUENCE
  Primer sequence for ChIP
tactagcggttttacgggcg

SEQ ID NO: 32 - DNA, 24 bp, ARTIFICIAL SEQUENCE
  Primer sequence for ChIP
tcgaacaggaggagcagagagcga

(19) United States
(12) Patent Application Publication
(10) Pub. No.: US 2018/0369203 A1
(43) Pub. Date: Dec. 27, 2018

(54) METHODS OF TREATING CANCER BY ADMINISTERING A MEK INHIBITOR IN COMBINATION WITH A PROTEASOME INHIBITOR

(71) Applicant: The Jackson Laboratory, Bar Harbor, ME (US)

(72) Inventors: Chengkai Dai, Bar Harbor, ME (US); Zijian Tang, Bar Harbor, ME (US)

(73) Assignee: The Jackson Laboratory, Bar Harbor, ME (US)

(21) Appl. No.: 15/742,725

(22) PCT Filed: Aug. 11, 2015
(86) PCT No.: PCT/US2015/044662

§ 371 Date: Jan. 8, 2018

Related U.S. Application Data
(60) Provisional application No. 62/190,563, filed on Jul. 9, 2015.

(51) Int. Cl. A61K 31/4184; A61K 31/69; A61P 35/00; C12Q 1/6886

(52) U.S. Cl. CPC A61K 31/4184; A61K 31/69; A61K 2300/00; C12Q 1/6886; A61P 35/00

(57) ABSTRACT

Presently disclosed are methods of treating cancer comprising administering a MEK inhibitor in combination with a proteasome inhibitor. In some embodiment, the cancer is a solid tumor. In some instances, the cancer has at least one mutation chosen from a NF1, RAS (including N-, K-, and H-RAS), RAF (including A-, B-, and C-RAF), and MEK (including MEK1 and MEK2) mutation. In some embodiments, the cancer is resistant to treatment with at least one of a proteasome inhibitor or a MEK inhibitor. In some embodiments, the combination therapy produces a synergistic effect.

This invention was made with government support under grant 1DP2OD007070 awarded by the National Institutes of Health. The government has certain rights in the invention.

### FIELD

Methods of Treating Cancer with a MEK inhibitor in combination with a proteasome inhibitor

### BACKGROUND

Cancer treatments, especially those for difficult to treat cancers like melanoma, require further advancement in order to achieve the clinical benefits that patients require to resume a healthy life without significant morbidity and mortality from the disease.

Additional research has been required to understand the mechanisms of action of cancer, as well as the mechanisms of action of different classes of pharmaceutical agents. Once these mechanisms of action are recognized, new combination therapy approaches may be employed to enhance the mutual effects of the pharmaceutical agents based on these mechanisms of action.

Following environmental challenges, cells stimulate production of heat-shock proteins (HSPs). This HSP induction is the hallmark of the heat-shock, or proteotoxic stress, response (PSR) (Lindquist, 1986). As molecular chaperones, HSPs facilitate folding, transportation, and degradation of other proteins (Morimoto, 2008). In guarding the proteome against misfolding and aggregation, the PSR preserves proteostasis (Balch et al., 2008).

In vertebrates heat shock transcription factors (HSFs) govern the PSR. Among them is HSF1, the master regulator of this response (Morimoto, 2008; Xiao et al., 1999). As a multi-step process, HSF1 activation entails trimerization, nuclear translocation, posttranslational modifications, and DNA binding (Morimoto, 2008). Yet, prior understanding of this process was incomplete.

The HSF1-mediated PSR antagonizes many pathological conditions, including hyperthermia, heavy-metal toxification, ischemia and reperfusion, and oxidative damage, and impacts aging and neurodegeneration (Dai et al., 2012a). HSF1, not surprisingly, acts as a longevity factor (Hsu et al., 2003). In contrast, our and others' work has revealed a pro-oncogenic role of HSF1 (Dai et al., 2007; Dai et al., 2012b; Jin et al., 2011; Meng et al., 2010; Min et al., 2007). Despite its dispensability under non-stress conditions, HSF1 is crucial for tumor cells' growth and survival (Dai et al., 2007). Nonetheless, the mechanisms underlying its activation in malignancy were unclear.

Herein we report that RAS-MEK-ERK signaling critically regulates the PSR. It is MEK that phosphorylates and activates HSF1. MEK inhibition destabilizes the proteome, provoking protein aggregation and amyloidogenesis. Combinatorial proteasome blockade potently augments this tumor-suppressive amyloidogenic effect.

MEK inhibitors were known in the art, as were proteasome inhibitors, yet there was no reason or motivation to combine them prior to the present invention and each treatment had its limitations in efficacy, including drug resistance.

With this understanding, we have developed a method of treating cancer comprising administering a MEK inhibitor in combination with a proteasome inhibitor. We have identified HSF1 as a new substrate for MEK, which suppresses the HSF1-mediated proteotoxic stress response. We have also found that MEK inhibition disrupts proteostasis and provokes tumor-suppressive amylodogenesis. We believe that combining a MEK inhibitor with a proteasome inhibitor will offer further advantages to either treatment alone.

### SUMMARY

In accordance with the description, a method of treating cancer comprises administering a MEK inhibitor in combination with a proteasome inhibitor.

In some embodiments, the cancer is a solid tumor, such as, but not limited to biliary (cholangiocarcinoma), bladder cancer, brain cancer, breast cancer, cervical cancer, colorectal cancer, endometrial cancer, epidermoid carcinoma, esophageal carcinoma, gallbladder cancer, gastric (stomach) cancer, glioblastoma, glioma, head and neck cancers, hepatocellular (liver) carcinoma, kidney cancer, lung cancer, mesothelioma, non-small cell lung cancer, ovarian, pancreatic cancer, pediatric malignancies, prostate cancer, renal cancer, sarcomas, skin cancer (including melanoma), small bowel adenocarcinoma, small cell lung cancer, testicular cancer, or thyroid cancer.

In some instances, the cancer has at least one mutation chosen from a NF1, RAS (including N-, K-, and H-RAS), RAF (including A-, B-, and C-RAF), and MEK (including MEK1 and MEK2) mutation. For example, the RAS mutation may be in at least codon 12, 13, or 61. In some embodiments, the RAF mutation is in at least codon 600. In some embodiments, the MEK1 mutation is at least P124S or S203K or the MEK2 mutation is at least Q60P.

In some embodiments, the MEK inhibitor is selumetinib (AZD6244), trametinib (GSK1120212), binimetinib (MEK162), PD-325901, cobimetinib, PD184352 (CI-1040), U0126-EtOH, refametinib (RDEA119), PD98059, BIX 02189, pimasertib (AS-703026), SL-327, BIX 02188, AZD8330, TAK-733, honokiol, or PD318088, PD0325901, WX-554, GDC-0623, E6201, RO4987655, RO5126766.

In some embodiments, the proteasome inhibitor is bortezomib, lactacystin, disulfiram, epigallocatcechin-3-gallate, salinosporamide A, carfilzomib, oprozomib (ONX 0912), delanzomib (CEP-18770), MLN9708, epoxomicin, MG132, ixazomib (MLN2238), PI-1840, or celastrol.

In some embodiments, the proteasome inhibitor and the MEK inhibitor are administered at a dosage that does not create a therapeutic benefit when either agent is administered alone. In some embodiments, selumetinib is administered at about 5 mg/Kg and bortezomib is administered at about 0.5 mg/Kg.

In some embodiments, the cancer is resistant to treatment with at least one of a proteasome inhibitor or a MEK inhibitor. In some embodiments, the combination therapy produces a synergistic effect. In some embodiments, the cancer is resistant to treatment with at least one of a proteasome inhibitor or a MEK inhibitor.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-O show that MEK and ERK oppositely regulate the PSR. (A) NIH3T3 cells were treated with HS at 43° C. for 30 min, 10 μM tubastatin A for 5 hr, 40 μM VER155008 for 1 hr, 500 nM MG132 for 1 hr, 200 nM 17-DMAG for 1 hr, and 2.5 mM azetidine for 15 min (B) The dual ELK1 reporter system, comprising a serum response element (SRE)-driven secreted embryonic alkaline phosphatase (SEAP) plasmid and a CMV-driven Gaussia luciferase (GLuc) plasmid, was transfected into HEK293T cells. After 24 hr, cells were treatments as in (A) and recovered overnight before measuring reporter activities (mean±SD, n=6, ANOVA). (C and D) NIH3T3 cells were treated with 20 μM U0126 or 20 nM AZD6244 for 3 hr followed by HS and 4-hr recovery. mRNA levels were quantitated by qRT-PCR (mean±SD, n=3, Student's t-test). (E) Immediately after HS, nuclear proteins of NIH3T3 cells treated as in (C) were extracted to measure HSF1-DNA binding by an ELISA-based assay (mean±SD, n=3, ANOVA). (F) HEK293T cells were transfected with dual HSF1 reporter plasmids, a heat-shock element (HSE)-driven SEAP plasmid and a CMV-GLuc plasmid. After 24 hr, cells were treated with 20 μM U0126, 20 nM AZD6244, 1 μM FR180204, or 100 nM Sch772984 for 3 hr followed by HS for 30 min and overnight recovery (mean±SD, n=6, ANOVA). (G) HEK293T cells were treated with different inhibitors overnight. (H)-(K) A LacZ or MEK isoform plasmid was co-transfected with dual HSF1 reporter plasmids into HEK293T cells transduced with lentiviral shRNAs. After 24 hr, cells were heat shocked at 43° C. for 30 min followed by overnight recovery (mean±SD, n=3, ANOVA). (L)-(O) HEK293T cells were transfected with siRNAs for 48 hr followed by transfection with dual HSF1 reporter plasmids for 24 hr before HS (mean±SD, n=6, ANOVA). See also FIG. 8.

FIGS. 2A-J show that ERK, MEK, and HSF1 form a stress-inducible protein complex. (A and B) After HS at 43° C. for 30 min, endogenous HSF1 proteins were precipitated from HEK293T cells. WCL: whole cell lysate; HC: heavy chain. (C) Endogenous MEK1-HSF1 interactions were detected by PLA in HeLa cells using a rabbit anti-MEK1 antibody and a mouse anti-HSF1 antibody. Scale bars: 50 μm for LM, 10 μm for HM. (D and E) Endogenous MEK-HSF1 interactions were detected by IP in HEK293T cells stably expressing shRNAs. (F) Endogenous MEK-HSF1 interactions were detected in HEK293T cells transfected with siRNAs. (G) Endogenous HSF1-MEK and HSF1-GFP-ERK1 interactions were detected in HEK293T cells transfected with indicated plasmids. (H) Schematic depictions of three possible scenarios. P: phosphorylation. (I) Immediately after HS, HSF1-ERK interactions were detected by co-IP. (J) Endogenous ERK-HSF1 interactions were detected in HEK293T cells stably expressing shRNAs. (K and L) Endogenous ERK-MEK and ERK-HSF1 interactions were detected by PLA in HeLa cells. Scale bar: 50 μm for LM, 10 μm HM. See also FIG. 9.

FIGS. 3A-O shows that MEK phosphorylates Ser326 to activate HSF1. (A and B) HSF1 Ser326 phosphorylation was measured by immunoblotting in HEK293T cells stably expressing shRNAs or transfected with MEK1^{DD} plasmid. (C) GFP or MEK1^{DD} plasmids were co-transfected with dual HSF1 reporter plasmids into HEK293T cells (mean±SD, n=5, Student's t-test). (D) Control or ERK-targeting siRNAs, A (siERK1_1 and siERK2_1) and B (siERK1_3 and siERK2_2), were transfected into HEK293T cells stably expressing shRNAs. (E) GFP or FLAG-HSF1 plasmids were co-transfected with dual HSF1 reporter plasmids into HEK293T cells stably expressing shRNAs (mean±SD, n=6, ANOVA). (F) FLAG-HSF1 plasmids were transfected into HEK293T cells stably expressing HSF1-targeting shRNAs. HSF1-DNA binding was measured after HS as described in FIG. 1E using anti-FLAG antibodies. The results were normalized against nuclear FLAG-HSF1 levels (mean±SD, n=3, Student's t-test). (G) FLAG-HSF1 proteins were detected in HEK293T cells treated with 20 μg/ml cycloheximide. Co-expressed GFP proteins served as internal controls. (H) 100 ng purified GST-MEK1 proteins were incubated with U0126 at RT for 20 min followed by incubation with 400 ng purified His-HSF1 proteins at RT for 30 min HSF1 phosphorylation was detected by immunoblotting. (I) ERK complexes precipitated from HEK293T cells were treated with U0126 or FR180204, followed by incubation with 400 ng His-HSF1, 400 ng GST-ERK1, or 1000 ng MBP proteins. (J) Inactive GST-ERK1 proteins were incubated with 100 ng GST-MEK1 and 400 ng His-HSF1 proteins at RT for 30 min (K) LacZ or GFP-ERK1 plasmid was co-transfected with MEK1^{WT} or MEK1^{T292A,T386A} plasmid into HEK293T cells stably expressing MEK-targeting shRNAs. (L) HSF1 Ser326 phosphorylation was detected in HEK293T cells transfected with indicated plasmids. (M) HSF1 activities were measured by the dual reporter system in HEK293T cells transfected with indicated plasmids (mean±SD, n=6, ANOVA). (N) WM115 cells were treated with 20 nM AZD6244 or 20 μM U0126 overnight. (0) HSF1 ChIP assays were performed using WM115 cells treated with DMSO or 20 nM AZD6244 overnight. The results were normalized against the values of IgG controls (mean±SD, n=3, ANOVA). See also FIG. 10.

FIGS. 4A-R show that MEK preserves proteostasis. (A) GFP and GFP-GR plasmids were co-transfected into HEK293T cells followed by treatments with 20 nM AZD6244, 20 μM U0126, or 200 nM 17-DMAG for 4 hr. (B) GFP-GR plasmids were co-transfected into HEK293T cells with HA-Ub-K48 plasmids, which encode a mutant ubiquitin that can be conjugated to protein substrates only via lysine 48. Following treatments with 20 nM AZD6244 or 200 nM 17-DMAG for 4 hr, GFP-GR proteins were precipitated and ubiquitination was detected using anti-HA antibodies. (C) Denatured firefly luciferases were incubated with lysates of A2058 cells treated with DMSO or 20 nM AZD6244 (mean±SD, n=4, ANOVA). (D) A2058 cells were treated with 20 nM AZD6244, and ubiquitinated proteins were detected in both detergent-soluble and -insoluble fractions using Lys48-specific ubiquitin antibodies. (E) A2058 cells stably expressing LacZ or HSF1^{S326D} were treated with 20 nM AZD6244 for 8 hr. (F) C57BL/6J mice were i.p. injected with DMSO or AZD6244 three times a week for 2 weeks. S: spleen; K: kidney; L: liver. (G) Experimental procedures of MS-based quantitation of ubiquitinated peptides, two technical replicates per treatment. (H) Scatter plot of relative changes in peptide abundance between treated and control conditions. The green and red lines indicate 2.5-fold cutoffs. (I) The classification of the 68 proteins was performed using the PANTHER gene list analysis tool (pantherdb.org). (J) The Gene Ontology (GO) biological process enrichment analysis was performed using the web-based Enrichr software application. (K) Interaction network of the 68 proteins. Known and predicted protein interactions were derived from the STRING database (www.string-db.org), and the network was visualized using Cytoscape software. (L and M) V5-TOR1AIP2 or V5-RPL3 plasmids were co-transfected with HA-Ub-K48 plasmids into HEK293T cells. Following 20 nM AZD6244 treatment for 8 hr, proteins were precipitated with anti-V5 antibodies. (N) Following AZD6244 treatment, endogenous c-MYC proteins were precipitated from A2058 cells and immunoblotted with anti-ubiquitin antibodies. (O and P) V5-RPL15 and V5-RPL3 plasmids were co-transfected with HA-Ub-K48 plasmids into HEK293T cells stably expressing shRNAs. Cells were treated with 500 nM MG132 alone or co-treated with 20 nM AZD6244 for 8 hr. (Q) Endogenous RPL15 and RPL3 proteins were detected in A2058 cells treated with 20 nM AZD6244 alone or co-treated with 500 nM MG132. (R) Endogenous RPL15 and RPL3 proteins were detected in A2058 cells stably expressing LacZ or HSF1^{S326D} with AZD6244 treatment. See also FIG. 11.

FIGS. 5A-Y show MEK and proteasome inhibition provoke protein aggregation and amyloidogenesis. (A-B) WM115 cells treated with 20 nM AZD6244, 100 nM Bortezomib, or both for 24 hr were stained with Lys48-specific ubiquitin antibodies. Arrowheads mark ubiquitin-positive aggregates. Scale bar: 10 μm. Amounts of aggregates per cell were quantitated using ImageJ (median, n≥100, ANOVA). (C-F) Following transfection with polyQ79 plasmids alone or with both polyQ79 and HSF1^{S326D} plasmids for one day, HEK293T cells were treated with inhibitors as described in (A-B). Cells were either analyzed for aggregate size or stained with 10 μM ThT. (G) Treated tumor cell lines were stained with 10 μM ThT. Geometric means were used to calculate fold changes in ThT fluorescence intensity and the log 2(FC) values were presented as a heat map. (H) HEK293T cells were transfected with LacZ or polyQ79 plasmid. Following treatments, AOs were quantitated by ELISA using A11 antibodies (mean±SD, n=3, ANOVA). (I) Intrinsic AOs were detected in human tumor cell lines (mean±SD, n=3, ANOVA). U and K) A2058 cells stably expressing LacZ or HSF1^{S326D} were treated for 24 hr. Amyloids were quantitated by ELISA (mean±SD, n=3, Student's t-test). (L) 20 μM synthetic Aβ1-42 peptides were incubated at RT with gentle shaking with 20 μg lysates of A2058 cells treated with inhibitors. AF formation was monitored by ThT binding (mean±SD, n=3, ANOVA). (M) For TEM studies (left panel 80,000×, right panel 200,000×), 20 μM synthetic Aβ1-42 peptides were incubated with A2058 cell lysates in PBS at 37° C. with gentle shaking for 2 days. Scale bars: 100 nm. (N) HEK293T cells stably expressing different shRNAs were stained with 10 μM ThT. (O and P) After pre-incubation with 10 μM ThT for 6 hr, A2058 cells were treated for 24 hr. Amyloids were quantitated (mean±SD, n=3, Student's t-test). (Q and R) After pre-incubation with 10 μM ThT for 6 hr or transfection with 100 ng A11 antibodies using JBS-Proteoducin for 16 hr, A2058 cells were treated for 24 hr. Viable cells were quantitated by CellTiter® blue (mean±SD, n=6, Student's t-test). (S and T) A2058 cells stably expressing LacZ or HSF1^{S326D} were treated with DMSO or 20 nM AZD6244. Viable cells were quantitated (mean±SD, n=6, ANOVA). Relative changes in viable cells after treatment were calculated by normalizing the values of AZD6244-treated cells against the values of DMSO-treated cells at each time point. (U)-(W) Following treatments with 20 nM AZD6244, 100 nM Bortezomib, or both for 24 hr, AOs were quantitated in primary MEFs and human cells (mean±SD, n=3, ANOVA). (X and Y) Cells were treated with 50 μM Q-VD-OPh overnight and AOs were quantitated (mean±SD, n=3, Student's t-test). See also FIG. 12.

FIGS. 6A-N show combined MEK and proteasome inhibition exerts potent tumor-suppressive effects. (A) After treatments with 20 nM AZD6244, 100 nM Bortezomib, or both for 24 hr, viable cells were quantitated by CellTiter® Blue (mean±SD, n=6, ANOVA). (B and C) 1×10⁶ A2058 cells were s.c. injected into NOD/SCID mice. After 7 days, mice were treated with DMSO, 5 mg/Kg AZD6244, 0.5 mg/Kg Bortezomib, or the combination via i.p. injection three times a week. Tumor volumes were measured using a caliper weekly (mean±SEM, ANOVA). Tumor growth curves were fitted to exponential growth models to derive tumor-doubling time (DT). Kaplan-Meier survival curve was plotted for each group (Log-rank test). (D) Proteins were detected by immunoblotting, 3 tumors per group. (E) Tumor lysates were used to quantitate AOs, 5 tumors per group (mean±SD, n=3, ANOVA). (F) Tumor lysates were used to seed Aβ1-42 peptides, 5 tumors per group (mean±SD, n=3, ANOVA). (G-H) Tumor sections were stained with CR, 5 tumors per group. Ten random fields were taken for each section. Scale bar: 50 μm. Total CR fluorescence in each field was quantitated using ImageJ and normalized against total nuclei (median, n=50, ANOVA). (I) Following CR staining, tumor sections were visualized under polarized light microscopy. Scale bar: 50 μm. (J) Following staining with AF-specific antibodies (OC), sections of tumors receiving combined treatment were further stained with CR. Scale bar: 50 μm. (K) 1×10⁶ A2058 cells stably expressing firefly luciferase transgene were i.v. injected into NOD/SCID mice. Treatments were initiated one day after as described in (B) for 6 weeks. Body weights were monitored weekly (mean±SD, n=10, ANOVA). (L) Detection of metastases by in vivo bioluminescence imaging. (M) Representative micrographs illustrate metastatic melanomas in the lung, skeletal muscle, pelvic adipose tissue, and ovary. T: tumors; L: lung; M: muscle; B: bone; A: adipose tissue; OF: ovarian follicle. Scale bar: 500 μm. (N) Combined MEK and proteasome inhibition prevents melanoma metastasis (Barnard's exact test). See also FIG. 13 and Table 2.

FIGS. 7A-H show amyloidogenesis suppresses tumor growth. (A) Sections of melanomas receiving combined treatment were stained with cleaved caspase 3 antibodies followed by CR staining Arrowheads and arrows indicate condensed and fragmented nuclei, respectively. Scale bar: 50 μm. (B) 1×10⁶ A2058 cells were s.c. injected into NOD/SCID mice. After 7 days, mice were treated with 1 mg/30 g CR via i.p. injection one day prior to combined treatment. Tumor volumes were measured weekly (mean±SD, ANOVA). (C) Lysates of CR-treated tumors exhibited absorbance at 498 nm, 3 tumors per group (mean±SD, n=3). Lysis buffer containing CR served as a positive control. (D) Kaplan-Meier survival curves were compared (Log-rank test). (E and F) Both detergent-soluble and -insoluble fractions of tumor lysates were used to quantitate amyloids, 3 tumors per group (mean±SD, n=3, Student's t-test). (G) Proteins were detected by immunoblotting, 3 tumors per group. (H) Schematic depiction of the interplay among MEK, ERK, and HSF1, and its role in regulating proteome stability. Balanced proteostasis suppresses toxic protein aggregation and amyloidogenesis, thereby facilitating tumorigenesis. See also FIG. 14.

FIGS. 8A-H illustrate that MEK and ERK inversely regulate the PSR. This figure is related to FIG. 1. (A) Cell viability is not impaired by transient exposure to proteotoxic stressors. NIH3T3 cells were treated with diverse stressors as described in FIG. 1A. Cell viability was measured by a Guava EasyCyte™ flow cytometer using Guava ViaCount™ reagents (mean±SD, n=5, Student's t test). (B) MEK blockade suppresses HSF1 activation triggered by diverse proteotoxic stressors. Following transfection with dual HSF1 reporter plasmids, HEK293T cells were treated with diverse stressors as described in FIG. 1B. After treatments, stressors were removed and cells were incubated with fresh medium for 24 hrs before measuring reporter activities (mean±SD, n=5, Student's t test). (C) MEK blockade impairs and ERK blockade enhances HSF1 activation. Following co-transfection of GFP or HSF1 plasmid with dual HSF1 reporter plasmids, HEK293T cells were treated with 20 μM U0126, 20 nM AZD6244, 1 μM FR180204, or 100 nM Sch772984 for 24 hrs before measuring reporter activities (mean±SD, n=6, ANOVA). (D) Both MEK and ERK blockade inactivate ELK1. HEK293T cells transfected with dual ELK1 reporter plasmids were treated with 20 μM U0126 20 μM U0126, 20 nM AZD6244, 1 μM FR180204, or 100 nM Sch772984 for 24 hrs before measuring reporter activities (mean±SD, n=5, ANOVA). (E and F) ERK1 knockdown does not reduce ERK2 mRNAs. Levels of ERK1 and ERK2 mRNAs were quantitated in HEK293T cells transfected with ERK1-targeting siRNAs by qRT-PCR (mean±SD, n=3, ANOVA). (G and H) ERK2 knockdown does not reduce ERK1 mRNAs. Levels of ERK2 and ERK1 mRNAs were quantitated in HEK293T cells transfected with ERK2-targeting siRNAs by qRT-PCR (mean±SD, n=3, ANOVA).

FIGS. 9A-F demonstrates that MEK physically interacts with HSF1. This figure is related to FIG. 2. (A and B) HSF1 is co-precipitated with MEK1 and MEK2. A FLAG-HSF1 plasmid was co-transfected with either HA-MEK1 or MEK2-V5 plasmid into HEK293T cells. HA-Raptor and LacZ-V5 plasmids served as negative controls. Following heat shock at 43° C. for 30 min, HSF1 proteins were immunoprecipitated using anti-FLAG affinity resin. (C and D) Validation of MEK1 and HSF1 antibodies. HeLa cells stably expressing scramble or MEK1/2-targeting shRNAs were immunostained with rabbit anti-MEK1 antibodies. HeLa cells stably expressing scramble or HSF1-targeting shRNAs were immunostained with mouse anti-HSF1 antibodies. Scale bar: 50 μm. (E) ERK suppresses HSF1 activation. GFP or GFP-ERK1 plasmid was co-transfected with dual HSF1 reporter plasmids into HEK293T cells. After 24 hrs, cells were heat shocked at 43° C. for 30 min followed by overnight recovery (mean±SD, n=6, Student's t test). (F) Validation of ERK1/2 antibodies. HeLa cells were transfected with control or ERK1/2-targeting (combined siERK1_3 and siERK2_2) siRNAs. Three days after transfection, cells were immunostained with rabbit anti-ERK1/2 antibodies. Scale bar: 50 μm.

FIGS. 10A-H illustrate that MEK phosphorylates HSF1 at Ser326. This figure is related to FIG. 3. (A) Validation of phospho-HSF1 Ser326 antibodies. Either FLAG-HSF1^{WT} or FLAG-HSF1^{S326A} plasmid was transfected into HEK293T cells stably expressing HSF1-targeting shRNAs. Following heat shock at 43° C. for 30 mins, phosphorylated and total HSF1 proteins were detected by immunoblotting. (B) MEK blockade impairs HSF1 Ser326 phosphorylation. NIH3T3 cells were treated with 20 μM U0126 for 3 hrs followed by heat shock at 43° C. for 30 mins HSF1 Ser326 phosphorylation was detected by immunoblotting. (C) MEK blockade impairs and ERK blockade enhances HSF1 Ser326 phosphorylation. Following overnight treatments with 20 μM U0126, 20 nM AZD6244, 1 μM FR180204, or 100 nM Sch772984, HSF1 Ser326 phosphorylation was detected by immunoblotting in HEK293T cells. (D-E) S326A mutation impairs HSF1 nuclear translocation. HEK293T cells were transfected with FLAG-HSF1^{WT} or FLAG-HSF1^{S326A} plasmids. Following heat shock at 43° C. for 30 mins, Cellular FLAG-tagged HSF1 proteins were immunostained with anti-FLAG monoclonal antibodies and images were captured by fluorescence microscopy. Scale bar: 50 μm for LM, 10 μm for HM. Cytoplasmic and nuclear fluorescent signals were quantitated using CellProfiler cell image analysis software and calculated as C/N ratios (median, n>150, ANOVA). (F) Acute MEK depletion diminishes HSF1 proteins. HEK293T cells were transiently transfected with scramble or MEK1/2-targeting shRNAs for 4 days. HSF1 proteins were detected by immunoblotting. (G) ERK-mediated HSF1 Ser307 phosphorylation depends on MEK. HEK293T cells stably expressing scramble or MEK1/2-targeting shRNAs were transfected with ERK1/2-targeting siRNAs as described in FIG. 3D. Levels of phosphor-HSF1 Ser307 were measured by immunoblotting. (H) HSF1 Ser326 phosphorylation inhibits Ser307 phosphorylation. HEK293T cells stably expressing HSF1-targeting shRNAs were transfected with FLAG-HSF1^{WT} or FLAG-HSF1^{S326D} plasmids. Following 20 nM AZD6244 treatment for 8 hrs, HSF1 Ser326 and Ser307 phosphorylation was detected by immunoblotting.

FIGS. 11A-P show that MEK regulates proteome stability. This figure is related to FIG. 4. (A) HSF1 stabilizes GR-GFP proteins. Both GR-GFP and HA-Ub-K48 plasmids were co-transfected with LacZ or FLAG-HSF1 plasmids into HEK293T cells stably expressing scramble or HSF1-targeting shRNAs. HA-Ub-K48 plasmids encode a mutant ubiquitin that can be conjugated to protein substrates only via lysine 48. GFP-GR proteins were precipitated and ubiquitination was detected by anti-HA immunoblotting. (B) HSF1 maintains cellular chaperoning capacity. Denatured firefly luciferases were incubated with proteins extracted from HEK293T cells stably expressing scramble or HSF1-targeting shRNAs that were transfected with LacZ or FLAG-HSF1 plasmids (mean±SD, n=5, ANOVA). (C) MEK blockade destabilizes GR proteins. HEK293T cells co-transfected with GFP and GR-GFP plasmids were treated with 200 nM 17-DMAG, 20 nM AZD6244, or 100 nM Bortezomib for 4 hrs. Levels of GFP and GR-GFP proteins were detected by immunoblotting. (D and E) MEK inhibition has no impacts on proteasomal activities. Following treatments with 20 μM U0126, 20 nM AZD6244, or 100 nM Bortezomib for 4 hrs, the chymotrypsin-like, trypsin-like, and caspase-like proteasomal activities in A2058 cells (D) and HEK293T cells (E) were measured in vitro using 25 μM Z-LLL-AMC, Boc-LRR-AMC, and Z-LLE-AMC substrates respectively (mean±SD, n=3, ANOVA). (F) MEK deficiency diminishes cellular chaperoning capacity. In vitro luciferase refolding assays were performed using lysates of HEK293T cells stably expressing scramble or MEK1/2-targeting shRNAs (mean±SD, n=5, ANOVA). (G) HSF1 suppresses protein ubiquitination. LacZ or FLAG-HSF1 plasmids were transfected into HEK293T cells stably expressing scramble or HSF1-targeting shRNAs. Lys48-specific protein ubiquitination was detected by immunoblotting in both detergent-soluble and -insoluble fractions. (H) AZD6244 promotes HSF1 ubiquitination. A2058 cells were treated with 20 nM AZD6244. Endogenous HSF1 proteins were immunoprecipitated and blotted with anti-ubiquitin antibodies. (I) MEK deficiency promotes protein ubiquitination. Lys48-specific protein ubiquitination was detected by immunoblotting in both detergent-soluble and -insoluble fractions of HEK293T cells that stably expressed scramble or MEK1/2-targeting shRNAs. (J) S326D mutation renders HSF1 proteins resistant to AZD6244-induced destabilization. A2058 cells stably expressing LacZ or HSF1^{S326D} were treated with 20 nM AZD6244 overnight. (K) Reproducibility of mass spectrometry analyses. The histogram of distribution of coefficient of variation (CV) of technical duplicates was plotted. (L)-(N) MEK inhibition reduces ribosomal protein levels. V5-tagged ribosomal proteins and HA-Ub-K48 were co-expressed in HEK293T cells. Following treatment with 20 nM AZD6244 for 8 hrs, ribosomal proteins were immunoprecipitated with anti-V5 antibodies and ubiquitination of these ribosomal proteins was detected by immunoblotting with anti-HA antibodies. (O and P) MEK and HSF1 deficiency destabilize ribosomal proteins. Endogenous RPL15 and RPL3 proteins were detected in HEK293T cells stably expressing scramble, MEK1/2-targeting, or HSF1-targeting shRNAs with and without 500 nM MG132 treatment for 24 hrs.

FIG. 12A-U shows that MEK and proteasome inhibition disrupt proteostasis. This figure is related to FIG. 5. (A-B) AZD6244 depletes HSF1 proteins in melanoma cells. WM115 and A2058 cells were treated with 20 nM AZD6244, 100 nM Bortezomib, or both for 24 hrs. (C) Quantitation of protein aggregate size. Four days after transfection with LacZ or polyQ79 plasmids, detergent-insoluble fractions of HEK293T cells were extracted to quantitate particle sizes using a Multisizer™ 3 coulter counter. (D) HSF1 suppresses protein aggregation. HEK293T cells stably expressing scramble or HSF1-targeting shRNAs were co-transfected with polyQ79 and LacZ or FLAG-HSF1 plasmids for 2 days. Aggregate sizes were measured. (E and F) Detection of amyloids in intact cells by ThT and CR. HEK293T cells transfected with LacZ or polyQ79 plasmid were stained with 10 μM ThT (E) or 50 nM CR (F) and analyzed by flow cytometry. (G) Following treatments, endogenous amyloids in WM115 cells were detected by CR staining (H and I) HSF1 suppresses amyloidogenesis. HEK293T cells stably expressing scramble or HSF1-targeting shRNAs were transfected with either LacZ or HSF1 plasmid. Levels of amyloid oligomers and fibrils were quantitated by ELISA (mean±SD, n=3, ANOVA). (J) HSF1-deficient cells exhibit enhanced amyloid seeding capacity. 20 μM synthetic Aβ1-42 peptides were incubated with 20 μg lysates of HEK293T cells stably expressing scramble or HSF1-targeting shRNAs at RT with gentle shaking Amyloid formation was monitored by ThT binding (mean±SD, n=3, ANOVA). (K) Lysates of A2058 cells treated with MEK and proteasome inhibitors exhibit enhanced seeding capacities. Synthetic Aβ1-42 peptides were incubated with lysates of A2058 cells treated with DMSO, 20 nM AZD6244, 100 nM Bortezomib, and combined 20 nM AZD6244 and 100 nM Bortezomib. Amyloid fibrils were detected in both soluble and insoluble fractions by ELISA using amyloid fibril-specific (OC) antibodies (mean±SD, n=3, ANOVA). (L and M) PSMB5 knockdown impairs proteasomal function and enhances amyloid seeding. HEK293T cells were transfected with two independent PSMB5-targeting shRNAs. (N)-(R) Genetic inhibition of MEK and proteasome disrupts proteostasis and provokes amyloidogenesis. HEK293T cells were transduced with lentiviral shRNAs targeting MEK1/2, PSMB5, or both. Proteostasis disruption was evidenced by increased protein ubiquitination (N). Amyloidogenic effects were measured by in vitro amyloid seeding assays (O, mean±SD, n=3, ANOVA, and P) and quantitation of endogenous AOs and AFs levels (Q and R, mean±SD, n=3, ANOVA). For TEM studies (P, left panel 80,000×, right panel 200,000×), 20 μM synthetic Aβ1-42 peptides were incubated with cell lysates in PBS at 37° C. with gentle shaking for 26 hrs. Scale bars: 100 nm. Of note, seeding with lysates of scramble cells only resulted in formation of disordered short protofibrils. In contrast, seeding with lysates of MEK- or PSMB5-deficient cells generated a dense lattice of fiber-like structures or abundant mature fibrils. (S) CR improves the growth and survival of melanoma cells treated with MEK and proteasome inhibitors. A2058 cells were co-treated with 10 μM CR and different inhibitors for 24 hrs. Viable cells were quantitated by CellTiter-Blue® reagents (mean±SD, n=6, Student's t test). (T) Primary tissues are resistant to amyloidogenesis induced by MEK inhibition. C57BL/6J mice were treated with different doses of AZD6244 and levels of amyloid oligomers in different tissues were measured by ELISA (mean±SD, n=2, ANOVA). (U) Severe proteotoxic stress is capable of inducing amyloidogenesis in MEFs. NIH3T3 cells were treated with different concentrations of Bortezomib for 24 hrs. Levels of endogenous amyloid oligomers were quantitated by ELISA (mean±SD, n=3, ANOVA).

FIG. 13A-J illustrates combined MEK and proteasome inhibition disrupts proteostasis and impedes in vivo tumor growth. This figure is related to FIG. 6. (A) Combined AZD6244 and Bortezomib treatment potently suppresses melanoma growth. Tumors were dissected when mice were sacrificed and weighed (mean±SD, ANOVA). (B) Combined AZD6244 and Bortezomib treatment prevents body weight loss. Body weights were recorded before xenografting and at sacrifice. Tumor weights were subtracted from total body weights to derive net body weights after xenografting. Results are presented as body weight changes before and after xenografting (paired Student's t-test). (C-F) AZD6244 suppresses Bortezomib-induced PSR and aggravates protein ubiquitination. Quantitation of immunoblotting results in FIG. 6D (mean±SD, n=3, ANOVA). (G) Amyloid oligomer levels are inversely correlated with tumor weights. (H) Combined AZD6244 and Bortezomib treatment markedly enhances ThT staining of tumors. Nuclei were stained with SYTO62. Scale bar: 50 μm. (I) Combined AZD6244 and Bortezomib treatment does not induce amyloidogenesis in normal tissues. Amyloid oligomers were assayed in 3 mouse spleens per group by ELISA (mean±SD, n=3, ANOVA). (J) Combined AZD6244 and Bortezomib treatment aggravates protein ubiquitination but fails to induce apoptosis in mouse spleens. Lys48-specific protein ubiquitination and caspase 3 cleavage were measured by immunoblotting in 3 spleens per group.

FIG. 14A-B shows that CR treatment promotes tumor growth and antagonizes tumor suppression imposed by combined MEK and proteasome inhibition. This figure is related to FIG. 7. (A) CR treatment stimulates in vivo melanoma growth and renders melanomas partially resistant to combined MEK and proteasome inhibition. Tumors were harvested at sacrifice and weighed (mean±SD, n=10, ANOVA). (B) CR treatment exacerbates body weight loss in tumor-bearing mice. Body weights were recorded before xenografting and at sacrifice. To derive net body weights of melanoma-bearing mice, tumor weights were subtracted from total body weights. Results are presented as body weight changes before and after xenografting (paired Student's t-test).

### DESCRIPTION OF THE SEQUENCES

Table 1 provides a listing of certain sequences referenced herein.

TABLE 1
Description of the Sequences

| | | |
|---|---|---|
| | | SEQ |
| Description | Sequences | ID NO |

Primer Sequences for qRT-PCR

| | | |
|---|---|---|
| Ms_Hspa1a-F | ATGGACAAGGCGCAGATCC | 1 |
| Ms_Hspa1a-R | CTCCGACTTGTCCCCCAT | 2 |
| Ms_Hspb1-F | ATCCCCTGAGGGCACACTTA | 3 |
| Ms_Hspb1-R | GGAATGGTGATCTCCGCTGAC | 4 |
| Ms_Hspa41-F | TTCCTCAACTGCTACATCGCT | 5 |
| Ms_Hspa41-R | CCTGTCGCTGTACTCGTTGG | 6 |
| Ms_Hspd1-F | CACAGTCCTTCGCCAGATGAG | 7 |
| Ms_Hspd1-R | CTACACCTTGAAGCATTAAGGCT | 8 |
| Ms_Hspe1-F | AGTTTCTTCCGCTCTTTGACAG | 9 |
| Ms_Hspe1-R | TGCCACCTTTGGTTACAGTTTC | 10 |
| Ms_Hsph1-F | GGGCTAGACGTAGGCTCACA | 11 |
| Ms_Hsph1-R | CCACCATTTTTCATTGGGACCA | 12 |
| Ms_Cryab-F | GTTCTTCGGAGAGCACCTGTT | 13 |
| Ms_Cryab-R | GAGAGTCCGGTGTCAATCCAG | 14 |
| Ms_Actb-F | GGCTGTATTCCCCTCCATCG | 15 |
| Ms_Actb-R | CCAGTTGGTAACAATGCCATGT | 16 |
| Hs_ERK1_F | CTACACGCAGTTGCAGTACAT | 17 |
| Hs_ERK1_R | CAGCAGGATCTGGATCTCCC | 18 |
| Hs_ERK2_F | TACACCAACCTCTCGTACATCG | 19 |
| Hs_ERK2_R | CATGICTGAAGCGCAGTAAGATT | 20 |
| Hs_ACTB_F | CATGTACGTTGCTATCCAGGC | 21 |
| Hs_ACTB_R | CTCCTTAATGTCACGCACGAT | 22 |

Primer Sequence for ChIP

| | | |
|---|---|---|
| Hs_HSPA1A-F | GGCGAAACCCCTGGAATATTCCCGA | 23 |
| Hs_HSPA1A-R | AGCCTTGGGACAACGGGAG | 24 |
| Hs_HSPA4L-F | CTCCTTTCCCCGATCCTC | 25 |
| Hs_HSPA4L-R | CAACGGCTGCCCAAGAAG | 26 |
| Hs_HSPB1-F | GTCGCGCTCTCGAATTCAT | 27 |
| Hs_HSPB1-R | CCTCCCCATGCACTCCTC | 28 |
| Hs_HSPD1-F | GTGTAGACCTTTTAGCCGATGC | 29 |
| Hs_HSPD1-R | GTGCCAGTACAGTAGCAGTGG | 30 |
| Hs_GAPDH-F | TACTAGCGGTTTTACGGGCG | 31 |
| Hs_GAPDH-R | TCGAACAGGAGGAGCAGAGAGCGA | 32 |

### Description of the Embodiments

### I. Methods of Treating Cancer

A method of treating cancer comprises administering a MEK inhibitor in combination with a proteasome inhibitor. We have identified HSF1 as a new substrate for MEK, which activates the HSF1-mediated proteotoxic stress response. We have also found that MEK inhibition disrupts proteostasis and provokes tumor-suppressive amyloidogenesis. We believe that combining a MEK inhibitor with a proteasome inhibitor will offer further advantages.

In some embodiments, the cancer is a solid tumor. In some embodiments, the solid tumor is biliary (cholangiocarcinoma), bladder cancer, brain cancer, breast cancer, cervical cancer, colorectal cancer, endometrial cancer, epidermoid carcinoma, esophageal carcinoma, gallbladder cancer, gastric (stomach) cancer, glioblastoma, glioma, head and neck cancers, hepatocellular (liver) carcinoma, kidney cancer, lung cancer, mesothelioma, non-small cell lung cancer, ovarian, pancreatic cancer, pediatric malignancies, prostate cancer, renal cancer, sarcomas, skin cancer (including melanoma), small bowel adenocarcinoma, small cell lung cancer, testicular cancer, or thyroid cancer. In some embodiments, the solid tumor is melanoma.

The method may provide additional advantages when the cancer has at least one mutation. For instance, the cancer may have at least one mutation chosen from a NF1, RAS (including N-, K-, and H-RAS), RAF (including A-, B-, and C-RAF), and MEK (including MEK1 and MEK2) mutation.

A RAS mutation may be present in at least codon 12, 13, or 61. A RAF mutation may be in at least codon 600. A MEK1 mutation may be in at least P124S or S203K. A MEK2 mutation may be in at least Q60P. Mutations in patient samples may be determined using known and available techniques.

The MEK inhibitor may be chosen from, but is not limited to, selumetinib (AZD6244), trametinib (GSK1120212), binimetinib (MEK162), PD-325901, cobimetinib, PD184352 (CI-1040), U0126-EtOH, refametinib (RDEA119), PD98059, BIX 02189, pimasertib (AS-703026), SL-327, BIX 02188, AZD8330, TAK-733, honokiol, or PD318088, PD0325901, WX-554, GDC-0623, E6201, RO4987655, RO5126766.

The proteasome inhibitor may be chosen from, but is not limited to, bortezomib, lactacystin, disulfiram, epigallocatcechin-3-gallate, salinosporamide A, carfilzomib, oprozomib (ONX 0912), delanzomib (CEP-18770), MLN9708, epoxomicin, MG132, ixazomib (MLN2238), PI-1840, or celastrol.

In some embodiments, wherein the proteasome inhibitor and the MEK inhibitor are administered at a dosage that does not create a therapeutic benefit when either agent is administered alone. In some embodiments, the selumetinib may be administered at 5 mg/Kg and the Bortezomib may be administered at 0.5 mg/Kg.

The method may present additional advantages when the cancer is resistant to treatment with at least one of a proteasome inhibitor or a MEK inhibitor. In some embodiments, the combination therapy may produce a synergistic effect. In some embodiments, the cancer is resistant to treatment with at least one of a proteasome inhibitor or a MEK inhibitor (meaning a proteasome inhibitor administered without a MEK inhibitor and/or a MEK inhibitor administered without a proteasome inhibitor), yet the combination of the two agents overcomes the resistance that may be associated with one or both alone.

### II. Pharmaceutical Compositions and Administration

The MEK inhibitor and the proteasome inhibitor may be prepared in separate compositions or they may be formulated into a single combined dosage form. For example, the inhibitors may be prepared as a tablet or capsule. Both agents may be coformulated in a single tablet or capsule, as separate sections in a bilayer tablet or capsule, or in separate tablets or capsules. In another embodiment, the inhibitors may be prepared in a dry powdered form to be mixed with water for injection prior to administration through a parenteral route of administration. In such an embodiment, they may be coformulated in the same vial or they may be prepared separately for administration to the patient.

If the inhibitors are formulated separately, they may be administered at the same time or in sequential order, including on either the same day or different days.

### EXAMPLES

### Example 1. Experimental Procedures

A. Proximity Ligation Assay

Cells were fixed with 4% formaldehyde in PBS for 15 min at RT. After blocking with 5% goat serum in PBS with 0.3% Triton X-100, cells were incubated with a pair of rabbit and mouse primary antibodies 1:200 diluted in the blocking buffer overnight at 4° C. Following incubation with Duolink® PLA® anti-rabbit Plus and anti-mouse Minus probes (OLINK Bioscience) at 37° C. for 1 hr, ligation, rolling circle amplification, and detection were performed using Duolink® In Situ Detection Reagents Red (OLINK Bioscience). Nuclei were stained with Hoechst 33342. Signals were visualized using a Leica TCS SP5 confocal microscope.

B. CR and ThT Staining of Tumor Sections

Following deparaffinization and rehydration, tumor sections were stained with 0.5% CR in PBS at RT for 20 min followed by differentiation in alkaline solutions (0.01% NaOH, 50% alcohol). Nuclei were stained with either Hoechst 33342 or hematoxylin. Fluorescence was visualized using a Leica TCS SP5 confocal microscope and the birefringence visualized using a Leica DM5000B upright microscope equipped with polarized light filters. For ThT staining, sections were stained with 0.2% ThT in PBS at RT for 10 min, rinsed in 1% acetic acid for 2 min, and washed with ddH₂O for 3 times. Nuclei were stained with SYTO® 62 (Life Technologies).

C. Melanoma Xenograft Models

A2058 cells were s.c. injected into the left flanks of 9-week-old female NOD.CB17-Prkdc<scid>/J (NOD/SCID) mice (The Jackson Laboratory). For CR treatment, mice were i.p. injected with PBS or CR one day prior to combined AZD6244 and Bortezomib treatments. Tumor volumes were calculated following the formula 4/3πR³. For experimental metastasis, engineered A2058 cells were transplanted into 10-week-old female NOD/SCID mice via tail vein injections. All mouse experiments were performed under a protocol approved by The Jackson Laboratory Animal Care and Use Committee.

D. Statistical Methods

All statistical analyses were performed using Prism 6.0 (GraphPad software). Statistical significance: *p<0.05; **p<0.01; ***p<0.001.

E. Supplemental Experimental Procedures

1. Sequences

Primer sequences for qRT-PCR are provided in Table 1 (SEQ ID NOS: 1-22) and primer sequences for ChIP are provided in Table 1 (SEQ ID NOS: 23-32).

2. Cells, Tissues and Reagents

All tumor cell lines except WM115, WM278, and A2058 were described previously (Dai et al., 2007). WM115 and WM278 cells were a kind gift from Dr. Luke Whitesell. A2058 cells were purchased from ATCC. All cell cultures were maintained in DMEM supplemented with 10% fetal bovine serum. Primary human mammary epithelial cells (PHMC) were purchased from Lonza and cultured in complete mammary epithelial cell medium (ScienCell Research Laboratories) on poly-L-lysine-coated plates. Primary human Schwann cells (PHSC) were purchased from ScienCell Research Laboratories and cultured in complete Schwann cell medium (ScienCell Research Laboratories) on poly-L-lysine-coated plates.

Antibodies against HSP72 (ADI-SPA-812), HSP25 (ADI-SPA-801), HSP27 (G3.1), and phospho-MBP T98 (P12) were purchased from Enzo® Life Sciences; rat monoclonal HSF1 (10H8) antibody, mouse monoclonal HSF1 (E-4) antibody, rabbit HSF1 antibody (H-311), rabbit p-HSF1 Ser307, rabbit MEK1 antibody (C-18), rabbit MEK2 antibody (N-20), Ubiquitin antibody (P4D1)-HRP, and rabbit c-Myc antibody (N-262) were from Santa Cruz Biotechnology; antibodies against total MEK1/2 (D1A5), phospho-MEK1/2 S218/222 (41G9), total ERK1/2 (137F5), phospho-ERK1/2 T202/Y204 (D13.14.4E), phospho-MSK1 T581, MEK1 (61B12), cleaved caspase 3 Asp175 (D3E9), GFP (D5.1), and GST tag (91G1) were from Cell Signaling Technology; antibodies against phospho-MEK1 T292, phospho-MEK1 T386, and Lys48-specific ubiquitin (Apu2) were from Millipore; and Total MSK1 antibody, βActin antibody-HRP, GAPDH antibody-HRP, HA-antibody-HRP, and FLAG antibody-HRP conjugates were from GenScript. Antibodies against p-HSF1 Ser326 (EP1713Y), 6×His tag (GT359), RPL15, and RPL3 were from GeneTex. Mouse monoclonal anti-V5 antibody was from Life Technologies.

The following chemicals were purchased from commercial sources: U0126 (Cell Signaling Technology), FR180204 (Tocris Bioscience), (S)-MG132 (Cayman Chemical), VER155008 (Tocris Bioscience), 17-DMAG (LC Laboratories), Bortezomib (LC Laboratories), AZD6244 (ChemieTek), tubastatin A (ChemieTek), azetidine (Bachem Americas), and Q-VD-OPH (APExBio).

The following purified recombinant proteins were purchased from commercial sources: His-tagged human HSF1 proteins (Enzo Life Sciences); GST and GST-tagged active human MEK1 (SignalChem); GST-tagged inactive human ERK1 proteins (Life Technologies); and bovine myelin basic proteins (Sigma-Aldrich).

Cytoplasmic and nuclear fractions were prepared using the NE-PER Nuclear protein Extraction Kit from Thermo Scientific.

The plasmids used in this study include: pLenti6-LacZ-V5 and pLenti6-MEK2-V5 (generated from pDONR223 vectors via Gateway® LR reaction), pMCL-HA-MEK1 from Natalie Ahn (Addgene#40808), pMCL-HA-MEK1 T292A, T386A (generated by HA-MEK1 site-directed mutageneses), pGFP-ERK1 from Rony Seger (Addgene#14747), pHSE-SEAP and pSRE-SEAP from Clontech Laboratories Inc., pCMV-Gaussia luciferase from ThermoFisher Scientific Inc., HA-Q79-GFP from Junying Yuan (Addgene#21159), HA-Q79 (generated from HA-Q79-GFP plasmid by removing GFP sequence), pRK5-HA-Raptor from David Sabatini (Addgene#8531), pK7-GR-GFP from Ian Macara (Addgene#15534), pRK5-HA-Ubiquitin-K48 from Ted Dawson (Addgene#17605), pLX304-TOR1AIP2-V5 (DNASU#HsCD00436680), pLX304-RPL3-V5 (DNASU#HsCD00435139), pLX304-RPL15-V5 (DNASU#HsCD00439802), pLX304-RPS20-V5 (DNASU#HsCD00443007), pLX304-RPL24-V5 (DNASU#HsCD00442995), pBabe-FLAG-HSF1 from Robert Kingston (Addgene#1948), pBabe-FLAG-HSF1 S326A and S326D (generated from FLAG-HSF1 by site-directed mutagenesis), and pLenti6-FLAG-HSF1 (generated via Gateway® LR reaction).

3. Real-Time Quantitative RT-PCR

Total RNAs were extracted using RNA STAT-60 reagent (Tel-Test, Inc.), and RNAs were used for reverse transcription using a Verso cDNA Synthesis kit (Thermo Fisher Scientific). Equal amounts of cDNA were used for quantitative PCR reaction using a DyNAmo SYBR Green qPCR kit (Thermo Fisher Scientific). Signals were detected by an ABI 7500 Real-Time PCR System (Applied Biosystems). The sequences of individual primers for each gene are listed in the Supplemental Materials.

4. Transfection and Luciferase Reporter Assay

Plasmids were transfected with TurboFect transfection reagent (Thermo Scientific). SEAP and luciferase activities in culture supernatants were quantitated using a Ziva® Ultra SEAP Plus Detection Kit (Jaden BioScience) and a Gaussia Luciferase Glow Assay Kit (Thermo Scientific), respectively. Luminescence signals were measured by a VICTORS Multilabel plate reader (PerkinElmer).

5. An ELISA-Based HSF1-DNA Binding Assay

Biotinylated ideal HSE (5′-CTAGAAGCTTCTAGAAGCTTCTAG-3′ (bolding indicates the nucleotide sequences recognized by HSF1, biotin added to the 5′ end)) oligonucleotides were self-annealed to form double-stranded DNA probes in annealing buffer (10 mM Tris, pH 7.5, 50 mM NaCl, 1 mM EDTA). To immobilize these probes for HSF1 binding, 100 μl of 500 nM biotinylated HSE probes diluted in PBS were added to Neutravidin-coated 96-well plates (Thermo Fisher Scientific) and incubated at 4° C. overnight. After washing with PBS once, wells were incubated with 200 μl SuperBlock blocking buffer (Thermo Fisher Scientific) at RT for 1 hr. After washing with 1×DNA binding buffer (10 mM Tris, 50 mM NaCl, 1 mM EDTA, 5% glycerol, pH7.5) once, 100 μl nuclear proteins diluted in 1×DNA binding buffer were added to each well and incubated at RT for 40 min. The captured protein-DNA binding was stabilized by immediately incubating the wells with 1% formaldehyde diluted in 1×DNA binding buffer at RT for 5 min Following washing 3 times with 1×DNA binding buffer, each well was incubated with 100 μl rabbit HSF1 antibodies (B7109, Assay Biotechnology Company, Inc.) diluted 1:1000 in SuperBlock blocking buffer at RT for 2 hrs. After TBS-T washing, each well was incubated with HRP-conjugated anti-mouse IgG secondary antibodies diluted in the blocking buffer at RT for 1 hr. Following extensive TBS-washing, colorimetric signals were developed using 1-Step Ultra TMB-ELISA substrate (Thermo Fisher Scientific).

6. siRNA and shRNA Knockdown

The negative control siRNA, which targets no known genes in human and mouse, was purchased from Thermo Fisher Scientific (D-001810-01). siERK1_1 (SIHK1207), siERK1_2 (SIHK1208), siERK1_3 (SIHK1209), siERK2_1 (SIHK1183), and siERK2_2 (SIHK1184) siRNAs were purchased from Sigma-Aldrich. siRNAs were transfected at 10 nM final concentration using Mission® siRNA transfection reagent (Sigma-Aldrich). Lentiviral shRNA targeting MEK1 (TRCN0000002329), MEK2 (TRCN0000007006), MEK1/2 (TRCN0000007007), and PSMB5 (TRC0000003918 and TRC0000003919) were purchased from Thermo Fisher Scientific. Lentiviral scramble and HSF1-targeting (hA6) shRNAs were described previously (Dai et al., 2007).

7. Immunoblotting and Immunoprecipitation

Whole-cell protein extracts were prepared in cold cell-lysis buffer (100 mM NaCl, 30 mM Tris-HCl pH 7.6, 1% Triton X-100, 30 mM NaF, 1 mM EDTA, 1 mM sodium orthovanadate, and Halt™ protease inhibitor cocktail from Thermo Scientific). Proteins were separated on SDS-PAGE gels and transferred to nitrocellulose membranes. Primary antibodies were applied in wash buffer overnight at 4° C. Peroxidase-conjugated secondary antibodies were applied at room temperature for 1 hr, and signals were visualized by SuperSignal West chemiluminescent substrate (Thermo Fisher Scientific), followed by exposure to films.

For immunoprecipitation, cells were lysed in CHAPS buffer (40 mM HEPES pH7.4, 120 mM NaCl, 2 mM EDTA, 0.3% CHAPS, 10 mM glyerophosphate, 50 mM NaF, and Halt™ protease inhibitor cocktail). Lysates were incubated with normal rabbit IgG (Santa Cruz Biotechnology), HSF1 antibodies (H-311, Santa Cruz Biotechnology), ERK1/2 antibodies (Cell Signaling Technology), or anti-FLAG G1 affinity resin slurry (GenScript) at 4° C. overnight. Protein G resin (GenScript) was used to precipitate immunocomplexes. After washing 3 times with lysis buffer, proteins were eluted from beads with 30 μl 0.1M glycine, pH2.5, before being subjected to SDS-PAGE.

8. In Vitro Kinase Assays

Immunoprecipitated ERK complexes were re-suspended in 1× in vitro kinase buffer (25 mM MOPS pH7.2, 12.5 mM β-glycerolphosphate, 25 mM MgCl₂, 5 mM EGTA, 2 mM EDTA, 0.25 mM DTT, and Halt™ protease inhibitor cocktail from Thermo Scientific), and incubated at RT for 20 min with U0126 or FR180204. Following addition of 100 μM ATP and purified recombinant His-tagged HSF1, GST-tagged ERK1, or bovine MBP proteins, kinase reactions were incubated at 25° C. for 30 min with gentle shaking in a thermomixer. Samples were boiled for 5 min to stop reactions.

9. Luciferase Refolding Assay

Recombinant firefly luciferase proteins (Promega) were denatured by incubating with denaturing buffer (25 mM HEPES, pH7.5, 50 mM KCl, 5 mM MgCl₂, 5 mM β-mercaptoethanol, and 6M guanidine HCl) at 37° C. for 20 min. To perform the refolding assay, 200 nM denatured luciferases diluted in refolding buffer (25 mM HEPES, pH7.5, 50 mM KCl, 5 mM MgCl₂, 10 mM DTT, and 1 mM ATP) were incubated with 5 mg/ml cell lysates extracted in passive lysis buffer (10 mM Tris-HCl pH7.5, 2 mM DTT, 1% Triton X-100, and 2 mM EDTA). At different time points, 20 μl refolding mixtures were removed and incubated with D-Luciferin (PerkinElmer) diluted in refolding buffer. Luminescence signals were measured by a VICTOR³ Multilabel plate reader (PerkinElmer).

10. Chromatin Immunoprecipitation

1×10⁷ cells were fixed with 1% formaldehyde for 8 min, and 125 mM glycine was added to stop the crosslinking After washing with cold PBS, cells were collected and lysed in cytoplasm extraction buffer (20 mM Tris-HCl, 85 mM KCl, 0.5% Triton X-100, pH8.0) for 10 min followed by centrifugation at 5,000 rpm for 5 min Pellets were further lysed in nuclei extraction buffer (50 mM Tris-HCl, 1% SDS, 10 mM EDTA, pH8.0) for 10 min and sonicated to shear chromatin to fragments with an average length of 500 bp. After centrifugation at 16,000×g for 15 min, supernatants were collected and 10% were saved as the inputs. To pre-clear the supernatants, 25 μl ChIP-grade protein G agarose beads (Cell Signaling Technology) were added and incubated at 4° C. for 3 hr. Pre-cleared supernatants were incubated with 4 μg rabbit anti-HSF1 antibodies (H-311, Santa Cruz Biotechnology) or 4 μg normal rabbit IgGs at 4° C. overnight followed by incubation with 25 μl ChIP-grade protein G beads at 4° C. for 3 hr. Beads were pelleted by brief centrifugation and sequentially washed with low-salt buffer, high-salt buffer, LiCl buffer, and TE buffer. After the final wash, 50 μl Chelex-100 resins were added to each sample and to the inputs, and the mixtures were boiled at 99° C. for 10 min. To reverse crosslinking, 40 μg proteinase K were added and incubated at 65° C. for 1 hr, and then boiled at 99° C. for 10 min After centrifugation at 16,000 g for 2 min, 2 μl supernatants were used for real time qPCR. IP signals were normalized against input signals.

11. Soluble and Insoluble Protein Fractionation

Equal numbers of cells were incubated with cell-lysis buffer containing 1% Triton X-100 on ice for 20 min. The crude lysates were first centrifuged at 500×g for 2 min at 4° C. The supernatants were further centrifuged at 20,000×g for 20 min at 4° C. The final supernatants and pellets were collected as detergent-soluble and -insoluble fractions, respectively. Insoluble fractions were further sonicated in 2% SDS at high intensity using a Bioruptor® Sonication System (Diagenode Inc.) for SDS-PAGE.

12. ThT and CR Staining for Flow Cytometry

After washing with PBS, cells were fixed by 4% formaldehyde at RT for 30 min Following fixation and PBS washing, cells were re-suspended in 2 ml penetration buffer (0.5% Triton X-100, 3 mM EDTA) and incubated on ice for 30 min Following washing with PBS, cells were stained with 10 μM ThT or 50 nM CR dissolved in PBS for 30 min ThT fluorescence was measured by a FACSCalibur™ flow cytometer (BD Biosciences).

13. Amyloid Oligomer and Fibril Quantitation by ELISA

To quantitate soluble amyloid prefibrillar oligomers, 20 μg soluble cellular proteins diluted in PBS were incubated for each well in a 96-well ELISA plate at 4° C. overnight followed by blocking (5% non-fat milk in PBS-T) at RT for 1 hr. Each well was incubated with 100 μl amyloid oligomer antibodies (A11, 1:1000 diluted in blocking buffer) at RT for 2 hr. After washing with PBS-T, goat anti-rabbit Ab HRP conjugates (1:5000 diluted in blocking buffer) were added to each well and incubated at RT for 1 hr. Following washing, 100 μl 1-Step™ Ultra TMB-ELISA substrates (Thermo Fisher Scientific) were added to each well.

To quantitate amyloid fibrils, detergent-insoluble proteins were extracted. Briefly, whole-cell lysates were centrifuged at 500×g for 2 min at 4° C. The supernatants were further centrifuged at 20,000×g for 20 min at 4° C. The final pellets were collected as detergent-insoluble fractions and solubilized by sonication for 10 min in PBS with 2% SDS. Following protein quantitation, 10 μg of solubilized proteins diluted in PBS were added to each well and incubated at 37° C. without cover overnight to dry the wells. The following steps were identical to the oligomer detection with the exception of the use of amyloid fibril antibodies (OC) as the primary Ab.

14. Transmission Electron Microscopy

Following in vitro seeding, amyloid fibrils were pelleted and re-suspended in distilled H₂O. One drop of fibril solution was placed on a 200-mesh carbon-coated nickel grid (Electron Microscopy Sciences). After 1 min, the remaining liquid was wicked. Immediately, a drop of 2% uranly acetate solution was placed on the grid for 1 min After wicking, the grids were air-dried and examined under a JEOL 1230 transmission electron microscope (JEOL USA Inc.) operating at 80 kV.

15. Bioluminescence Imaging

Before imaging, XenoLight RediJect D-luciferin (150 mg/kg) was i.p. injected into NOD/SCID mice that were previously injected with luciferase-expressing A2058 cells. Mice were anesthetized with isoflurane, and luminescence signals were recorded using a Xenogen IVIS® Lumina II system (Caliper Life Sciences). Images of both dorsal and ventral positions were captured. The total photon flux of each mouse was quantified using Living Image® software.

16. Measurement of Aggregate Size

Equal numbers of cells from different samples were lysed with cold cell lysis buffer. Following centrifugation at 20,000×g for 15 min at 4° C., detergent-insoluble pellets were further extracted with RIPA buffer 3 times. The final insoluble pellets were re-suspended in 10% SDS by pipetting and immediately subjected to aggregate sizing using a Multisizer™ 3 Coulter Counter equipped with a 20 μm aperture (Beckman Coulter).

17. Immunofluorescence Staining

Following fixation with 4% formaldehyde in PBS at RT for 15 min, cells were blocked with 5% goat serum in PBS containing 0.3% Triton X-100 at RT for 60 min and incubated with Lys48-specific ubiquitin Abs (Millipore, 1:500 dilution in blocking buffer) overnight at 4° C. For tumor sections, antigens were retrieved in 10 mM sodium citrate buffer followed by blocking. Sections were incubated with either cleaved caspase 3 Asp175 Abs (Cell Signaling Technology, 1:500 dilution in blocking buffer) or amyloid fibril (OC) Abs (StressMarq Biosciences, 1:200 dilution in blocking buffer) overnight at 4° C. After washing with PBS, sections were incubated with Alexa Fluor® 568 or 488 goat anti-rabbit IgG Abs (Life Technologies, 1:1000 dilution in blocking buffer). Following Hoechst 33342 nuclear counterstaining, fluorescent images were captured by a Leica TCS SP5 confocal microscope.

18. Ubiquitination Proteomics

A2058 cells were grown in 150 mm culture dishes and treated with DMSO or 20 nM AZD6244 for 8 hrs. 2×10⁸ cells receiving the same treatment were pooled and snap frozen in liquid nitrogen. Global quantitative analysis of cellular ubiquitination was conducted through the UbiScan® service (Cell Signaling Technology), which combines enrichment of ubiquitinated peptides by an ubiquitin branch (K-ϵ-GG) monoclonal antibody with liquid chromatograph tandem mass spectrometry (LC-MS/MS). Two technical replicates were analyzed for each treatment.

19. In Vitro Amyloid Seeding

Cells were suspended in 2% PBS and sonicated to prepare lysates. Seeding experiments were performed in 96-well black microplates, 100 μl reaction volume per well. Each reaction contained 20 μg cellular proteins diluted in 80 μl PBS and 10 μl of 200 μM synthetic human Aβ1-42 peptides (GenScript) dissolved in 0.01M NaOH. Reactions were incubated at RT with gentle shaking. To detect amyloid formation, 10 μl of 100 μM ThT (Sigma) dissolved in PBS were added to the reaction and fluorescence was measured at Ex450 nm/Em482 nm.

### Example 2. MEK and ERK Inversely Regulate the PSR

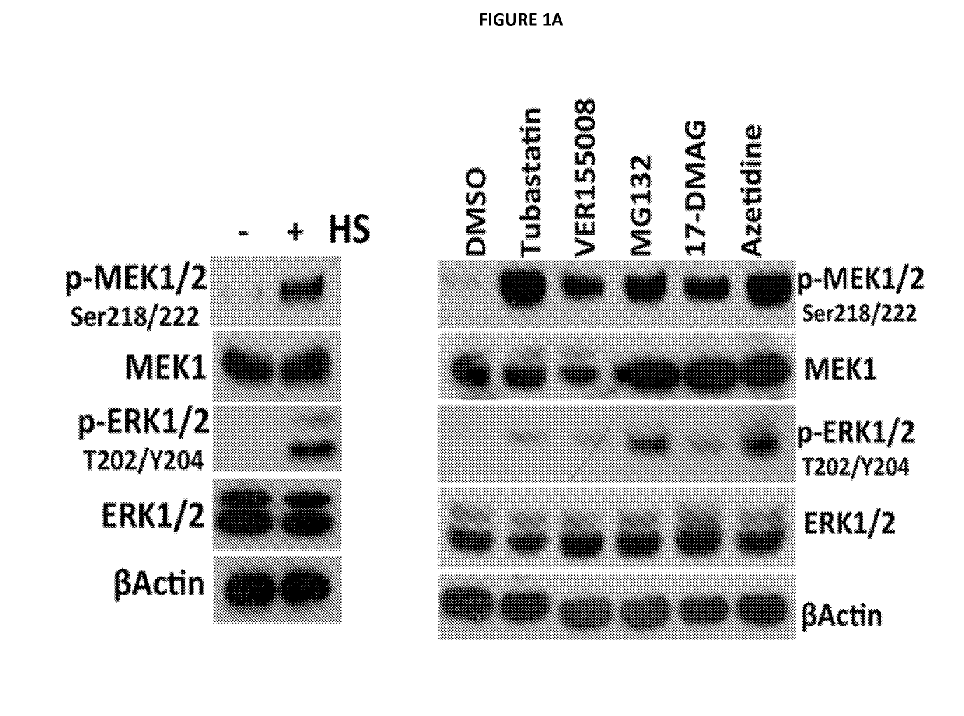

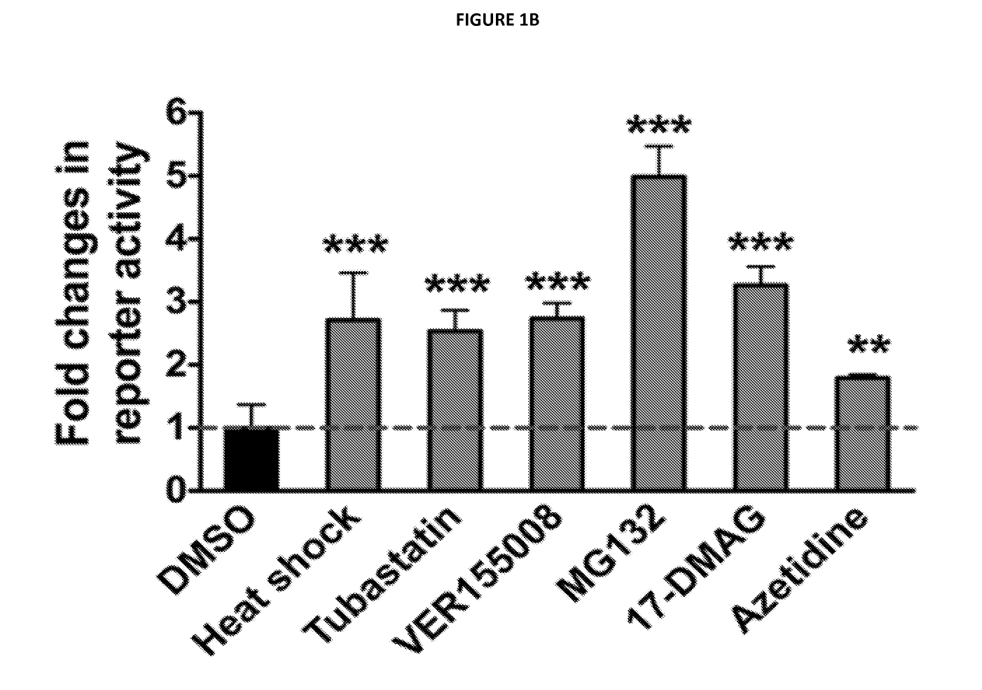

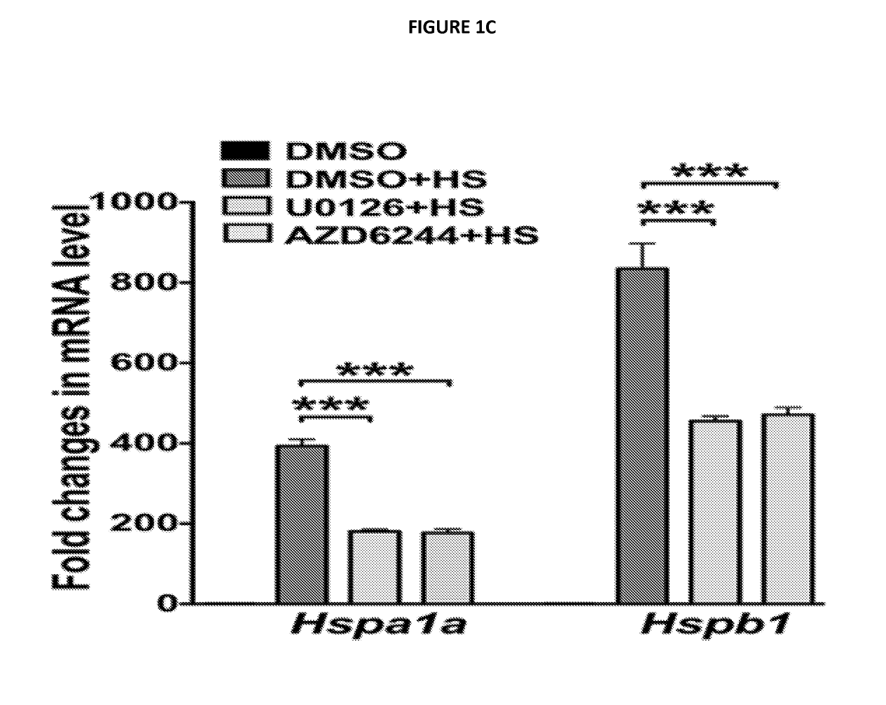

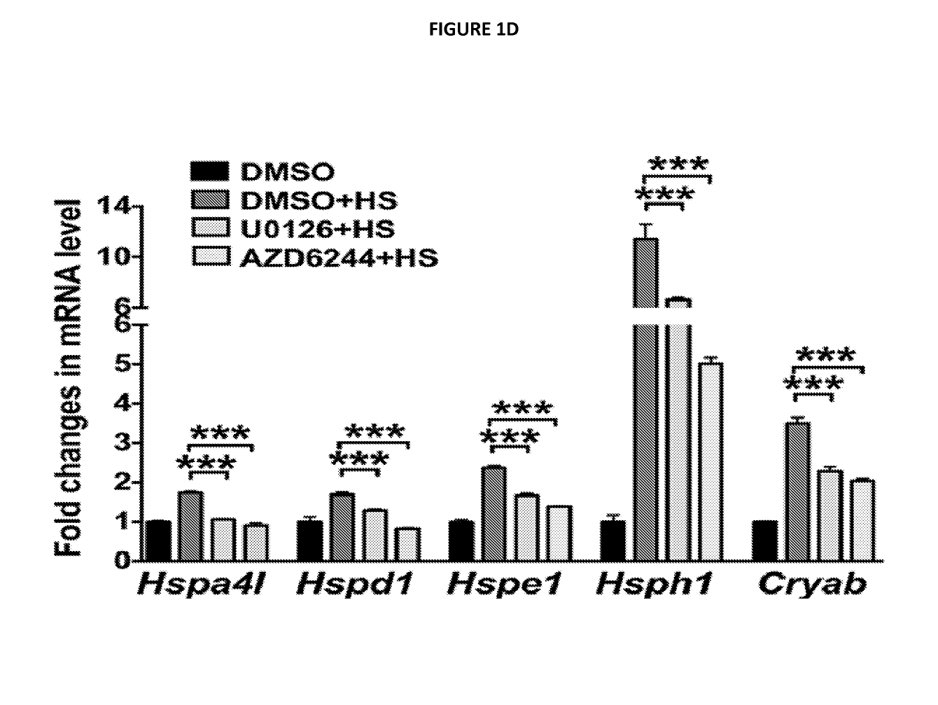

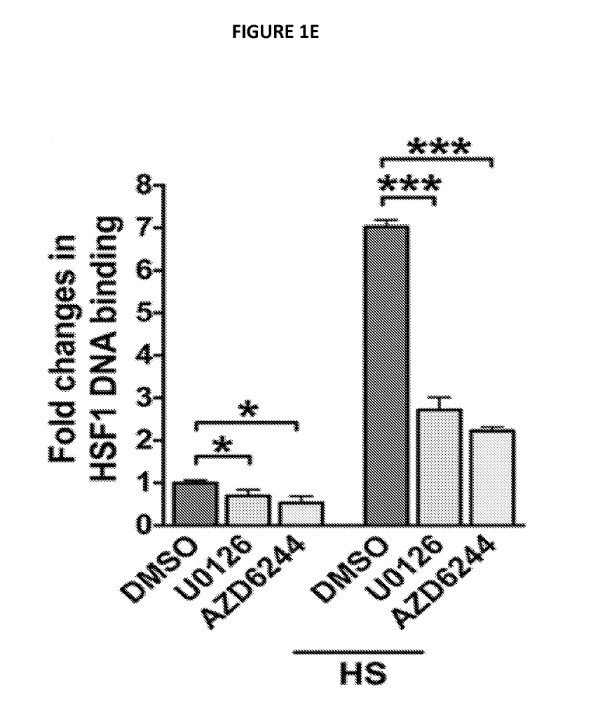

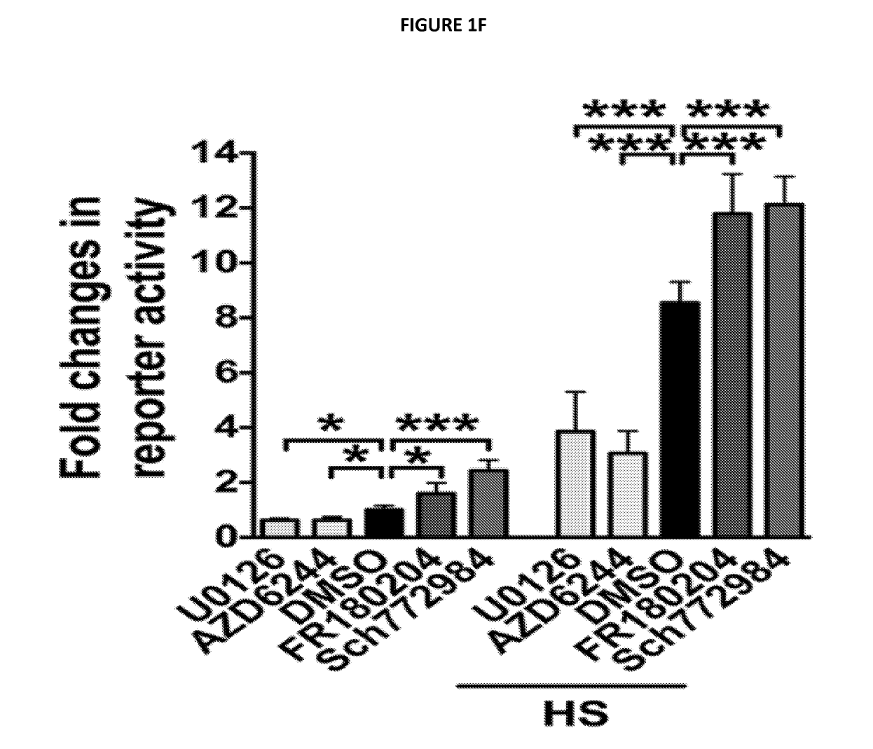

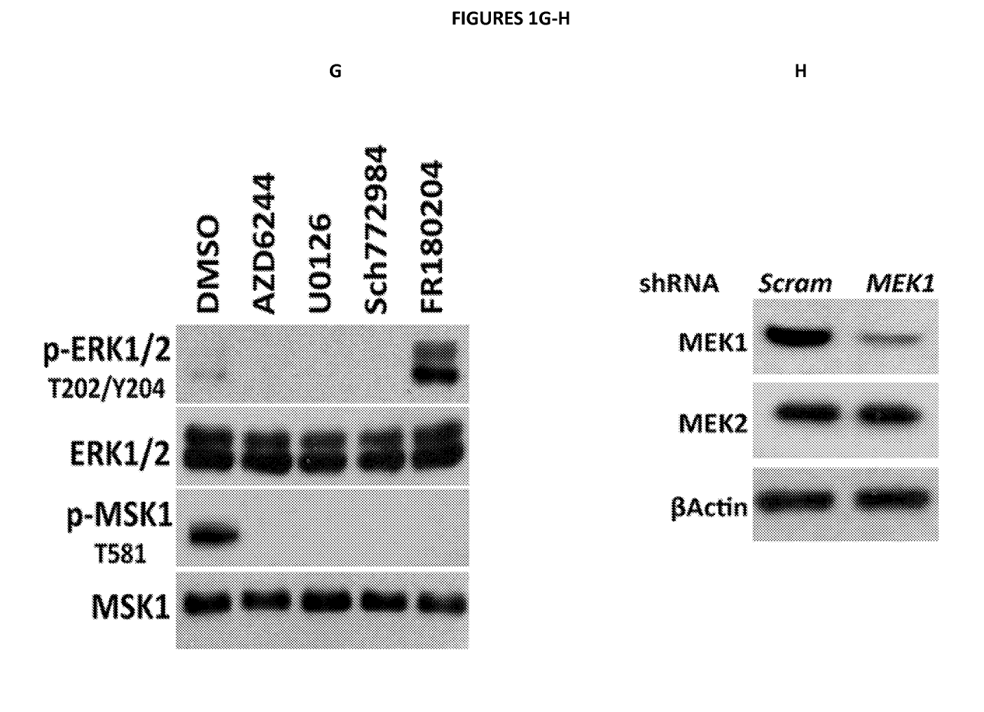

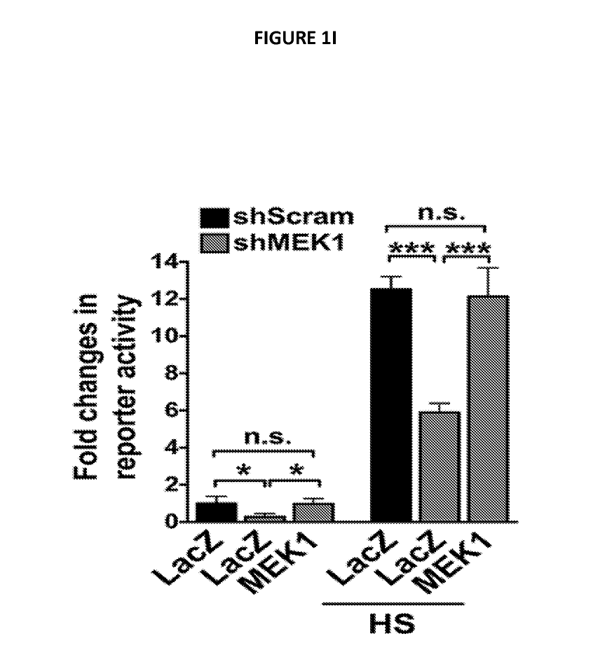

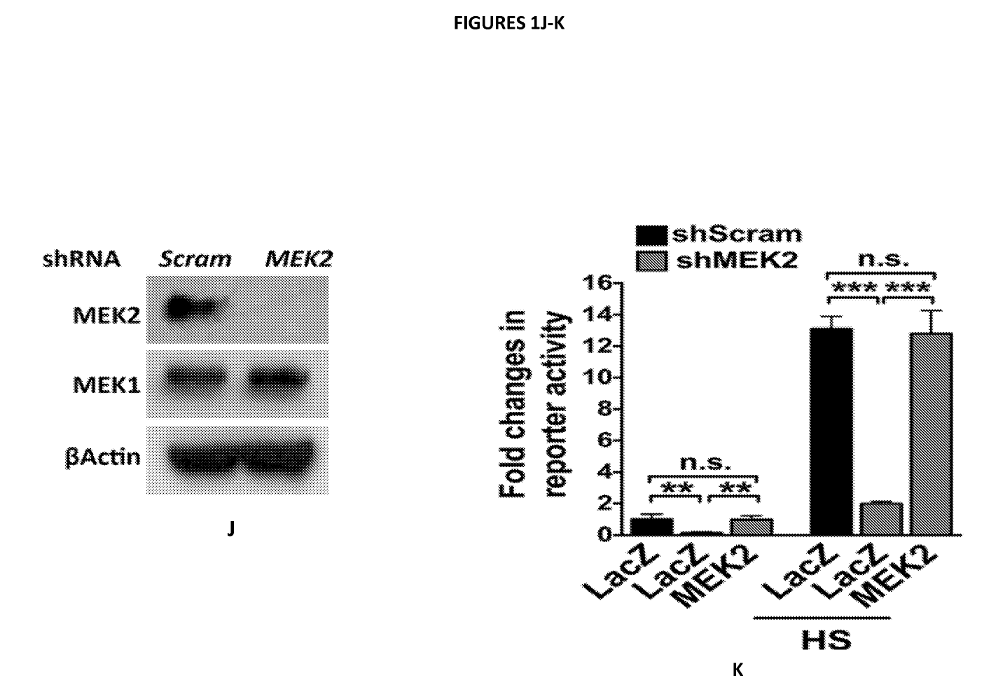

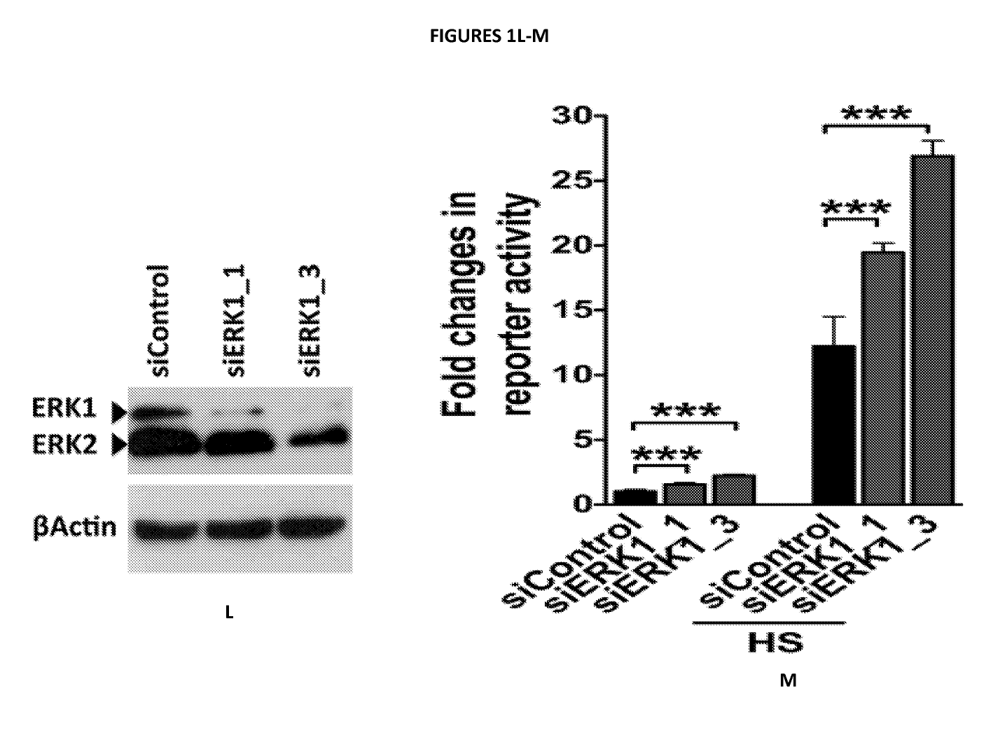

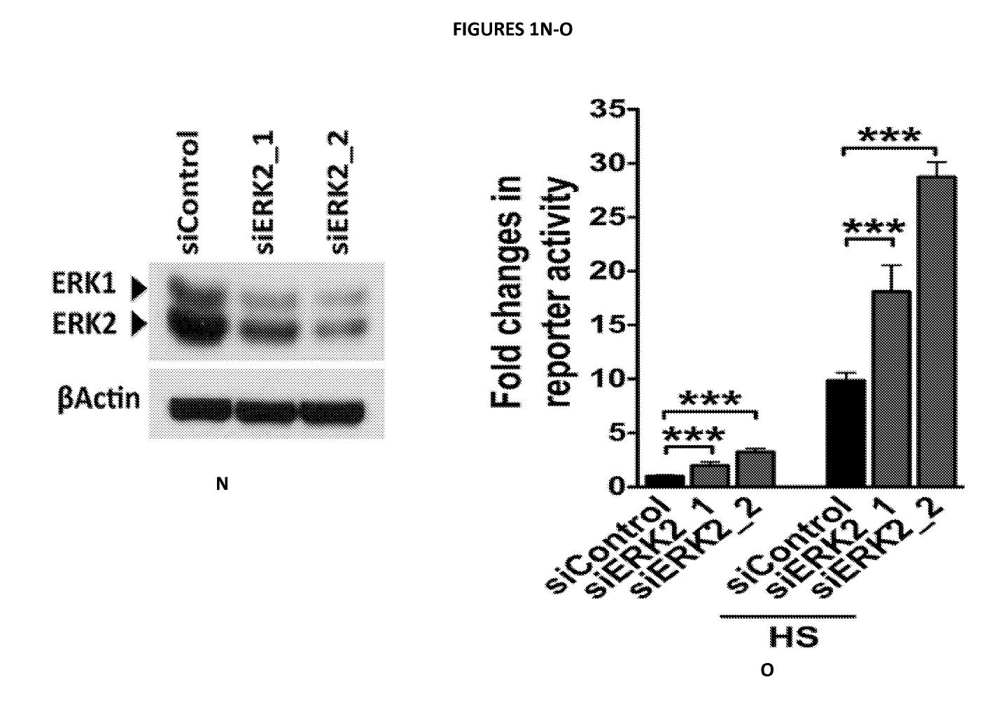

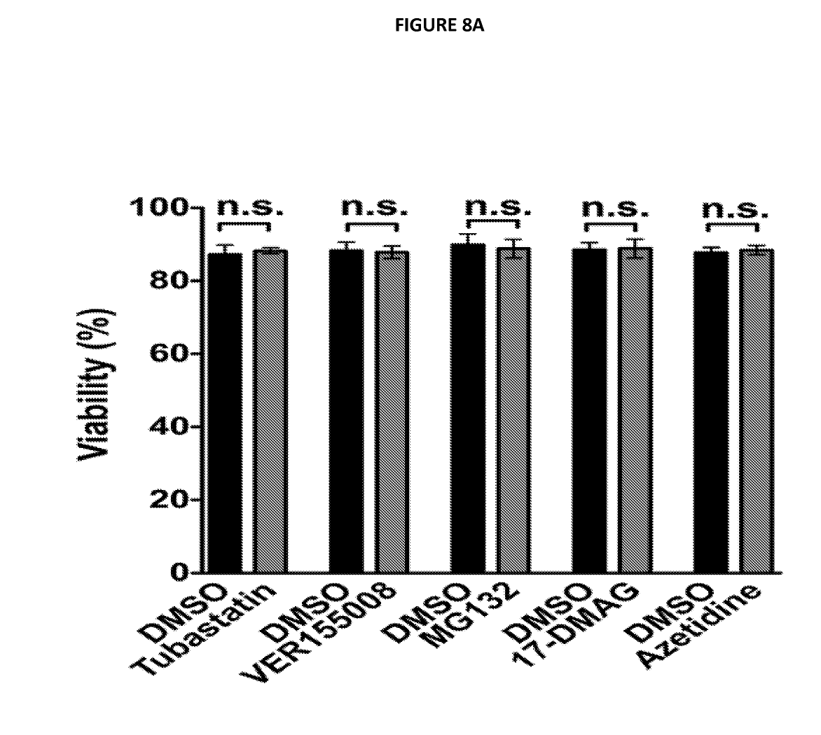

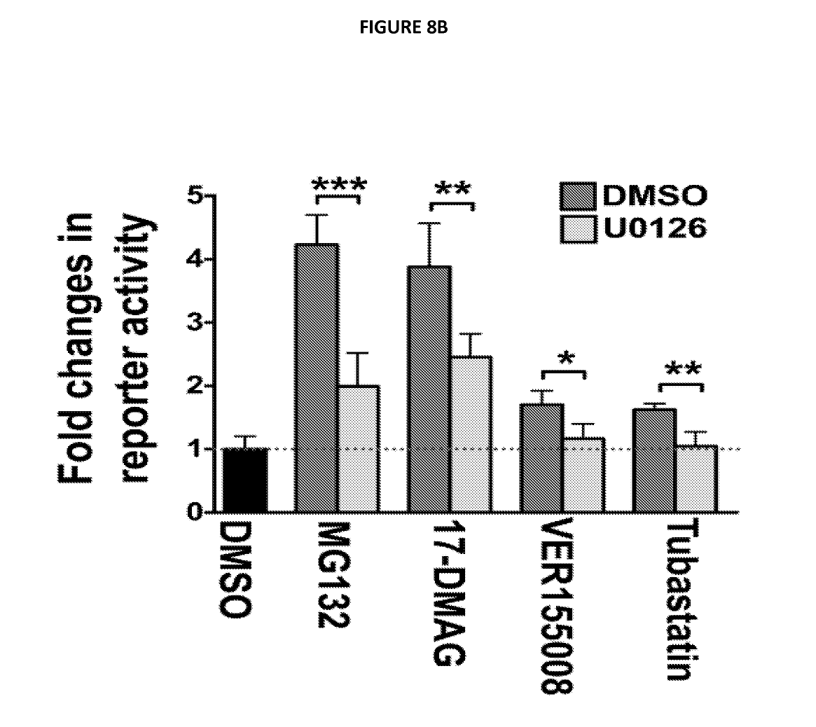

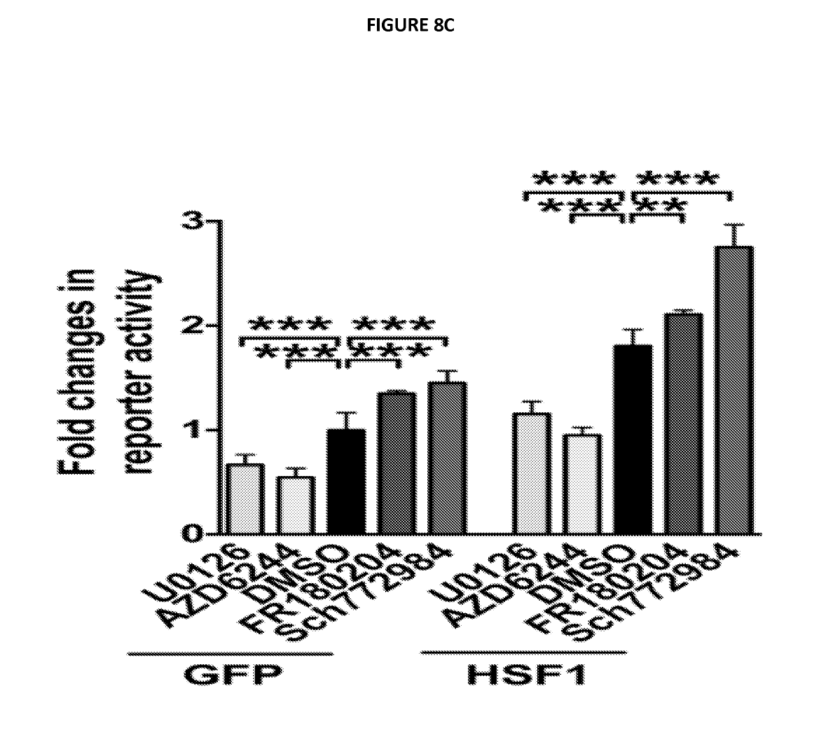

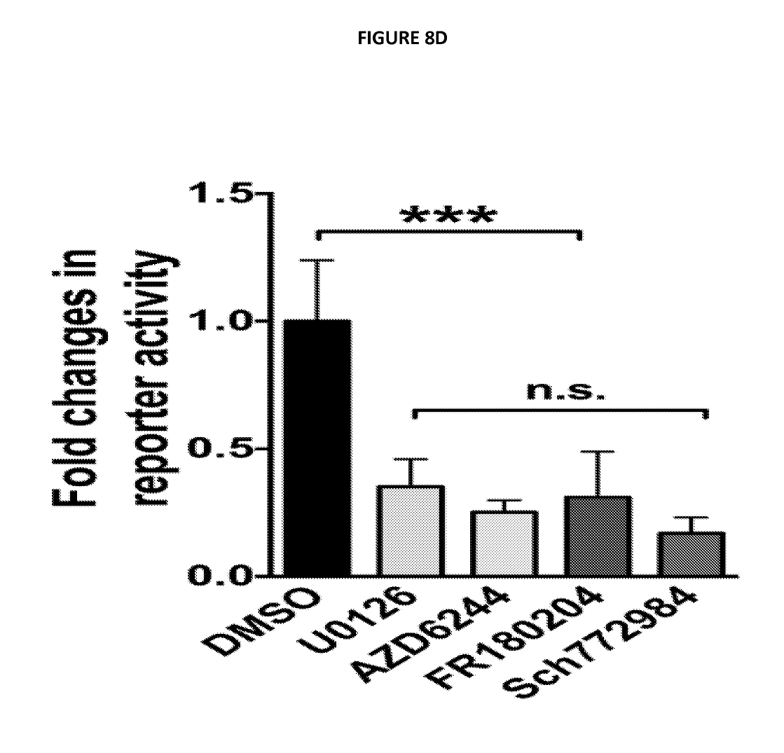

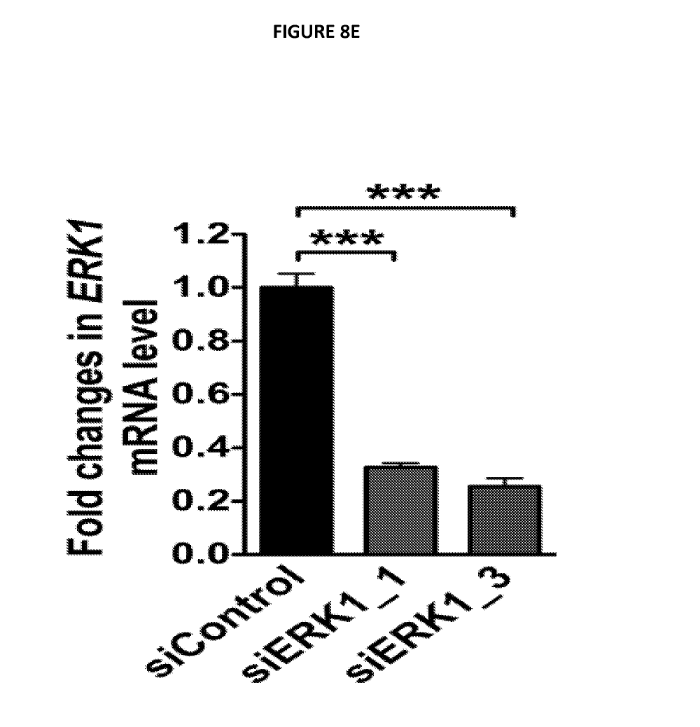

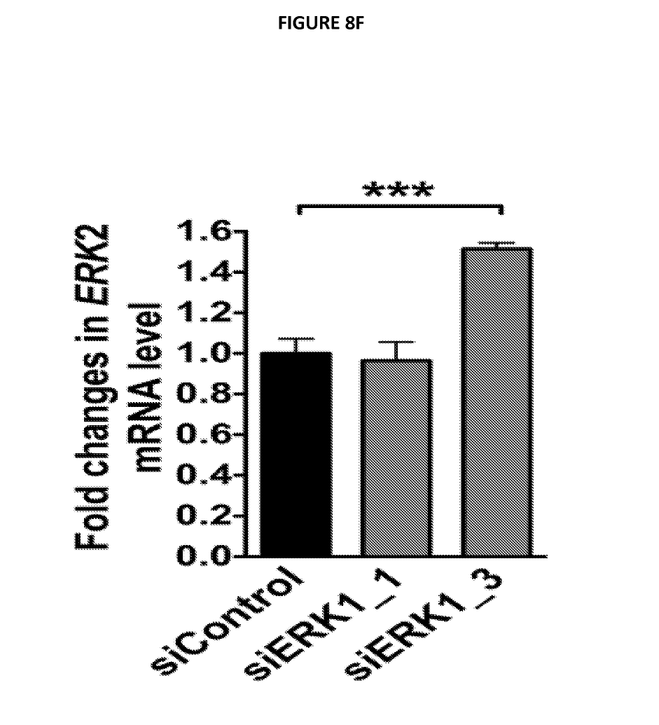

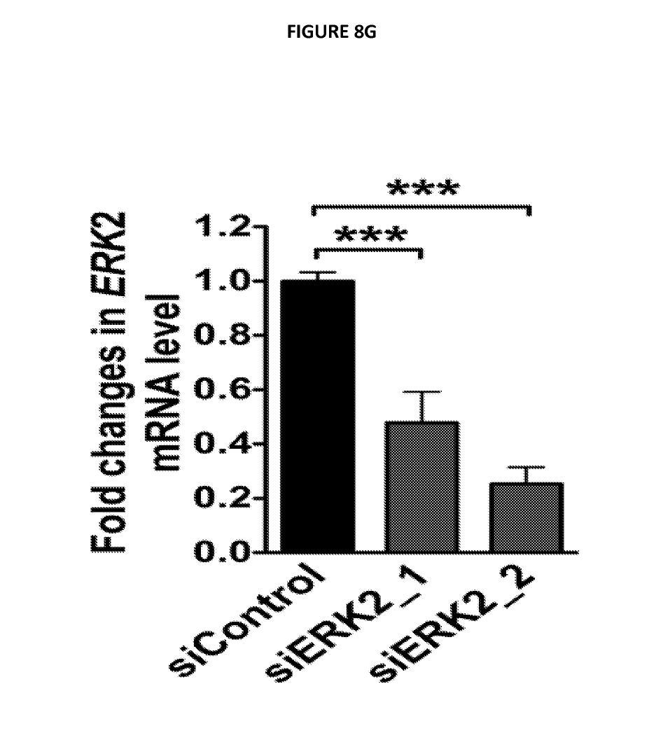

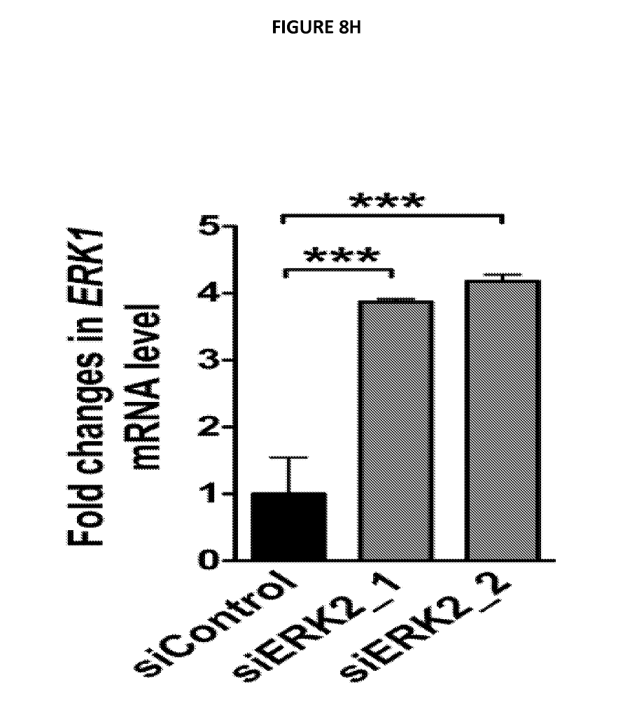

Phosphorylation notably impacts HSF1 activation (Guettouche et al., 2005), suggesting a key role of signaling pathways. To illuminate how such pathways regulate the PSR, we first examined their responses to stress, focusing on RAS-MEK-ERK signaling. To inflict proteotoxic stress, we applied stressors with diverse mechanisms of action, including heat shock (HS), proteasome inhibitor MG132, histone deacetylase 6 inhibitor tubastatin, amino-acid analog azetidine, and HSP inhibitors (17-DMAG for HSP90 and VER155008 for HSP70) (Kawaguchi et al., 2003; Massey et al., 2010; Morimoto, 2008; Neckers and Workman, 2012). Transient exposure to stressors did not impair cell viability (FIG. 8A), but elevated phosphorylation of MEK and ERK (FIG. 1A), two key components of this pathway. MEK Ser218/222 and ERK Thr202/Tyr204 phosphorylation signify their active state (Dhillon et al., 2007; Roux and Blenis, 2004). Congruently, all stressors activated ELK1 (FIG. 1B), a transcription factor downstream of ERK (Roux and Blenis, 2004).

To determine whether MEK-ERK signaling regulates the PSR, we employed U0126 and AZD6244, two specific MEK1/2 inhibitors (Favata et al., 1998; Yeh et al., 2007). Both inhibitors impeded the HS-induced transcription of Hsp genes, and impaired the DNA-binding capacity and transcriptional activation of HSF1 (FIGS. 1C-1F and 8B), suggesting that MEK activates the HSF1-mediated PSR. ERK, phosphorylated by MEK (Ahn et al., 1991), is widely recognized as the master effector of this pathway (Dhillon et al., 2007; Roux and Blenis, 2004). Surprisingly, ERK inhibitors, FR180204 and Sch772984 (Ohori et al., 2005; Morris et al., 2013), activated HSF1 (FIGS. 1F and 8C). Both MEK and ERK inhibitors impaired two ERK-mediated events—MSK1 phosphorylation and ELK1 activation (FIGS. 1G and 8D; Roux and Blenis, 2004). While MEK inhibitors reduced ERK phosphorylation, two ERK inhibitors showed distinct effects (FIG. 1G). Sch772984 suppressed ERK phosphorylation, likely due to ERK conformational changes that block MEK-mediated phosphorylation (Morris et al., 2013); conversely, FR180204 promoted ERK phosphorylation (FIG. 1G), suggesting feedback MEK activation.

The impacts of MEK and ERK inhibitors on HSF1 were validated via genetic depletions of MEK and ERK (FIGS. 1I-1O). While depletion of one ERK isoform diminished the other isoform at the protein level (FIGS. 1L and 1N), mRNA levels of the isoform not targeted were elevated (FIG. 8E-8H), suggesting posttranscriptional mechanisms underlying reduced proteins. These results not only pinpoint RAS-MEK-ERK signaling as a key regulator of the PSR, but also reveal divergent impacts of MEK and ERK on HSF1.

### Example 3. MEK Physically Interacts with HSF1

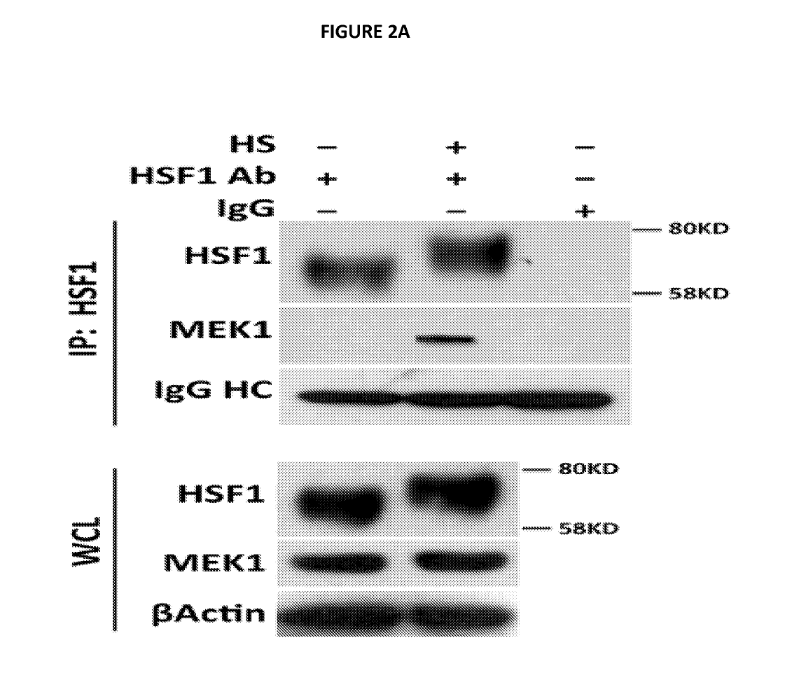

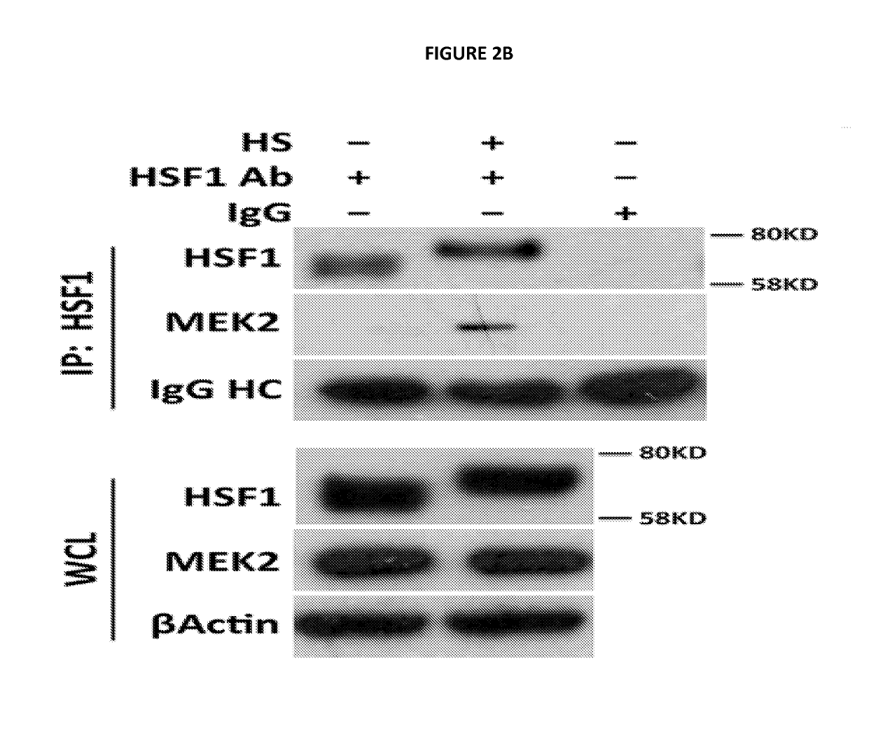

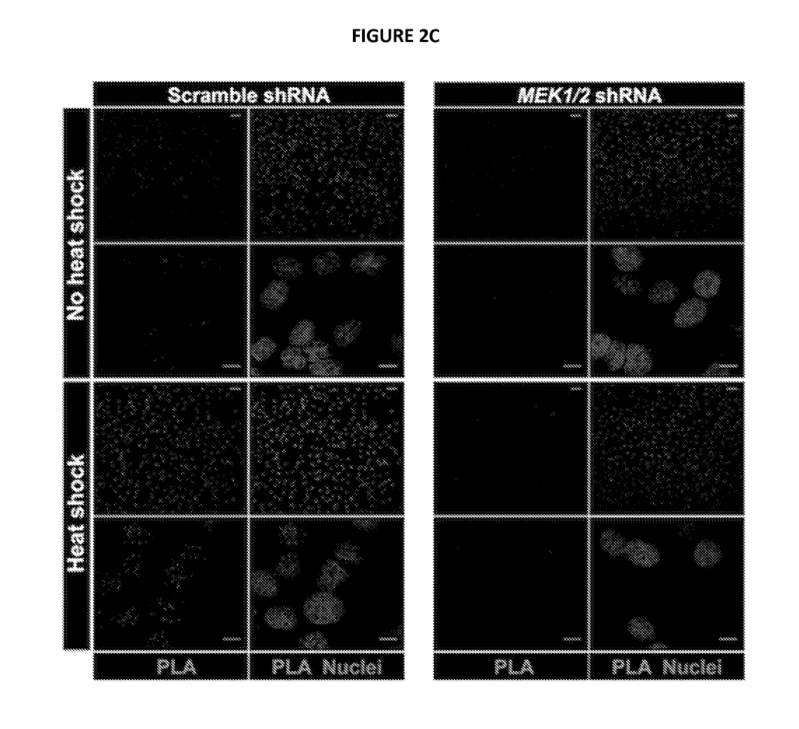

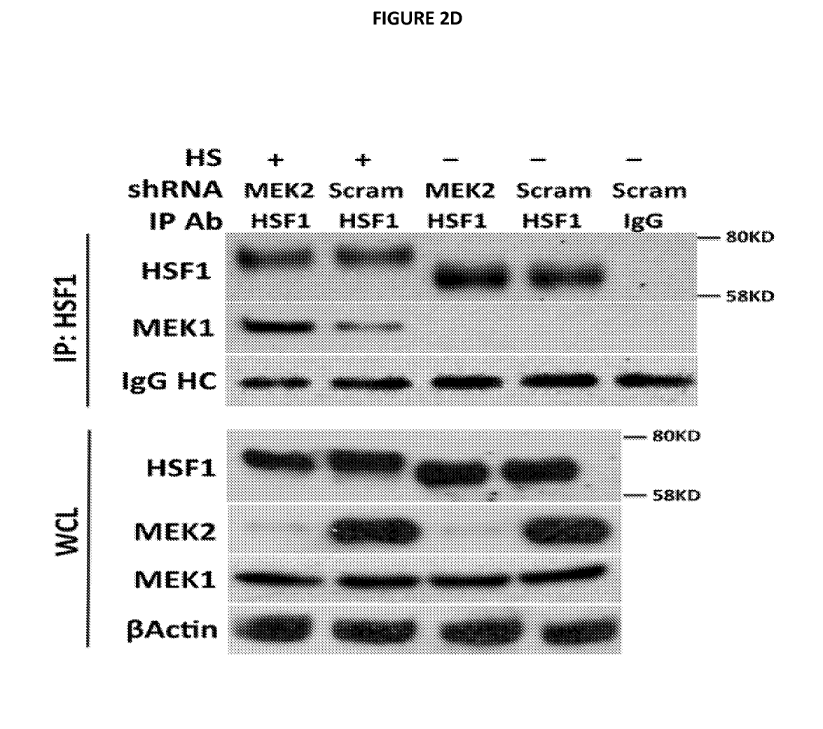

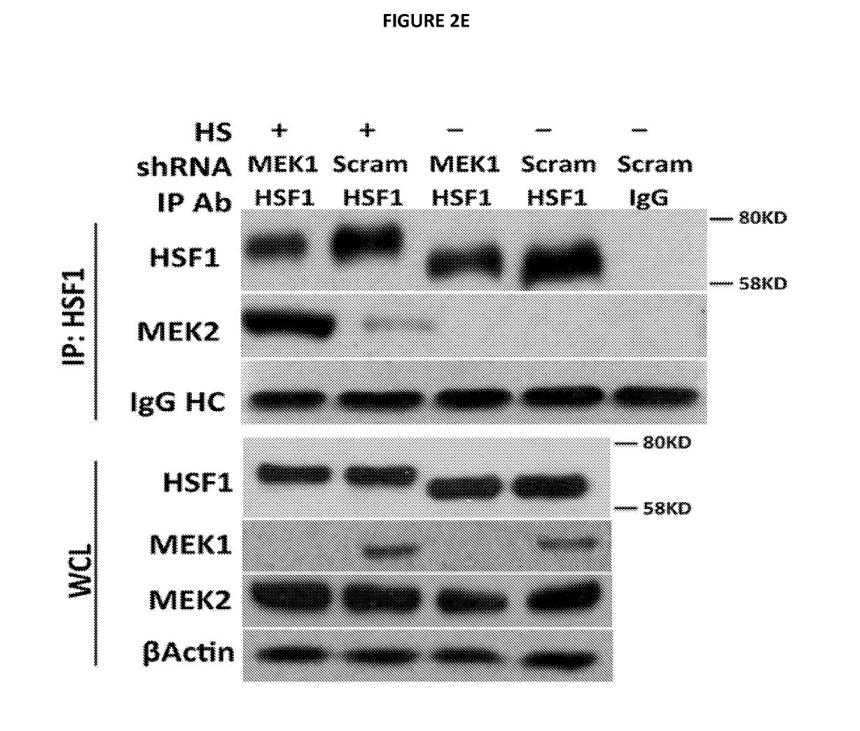

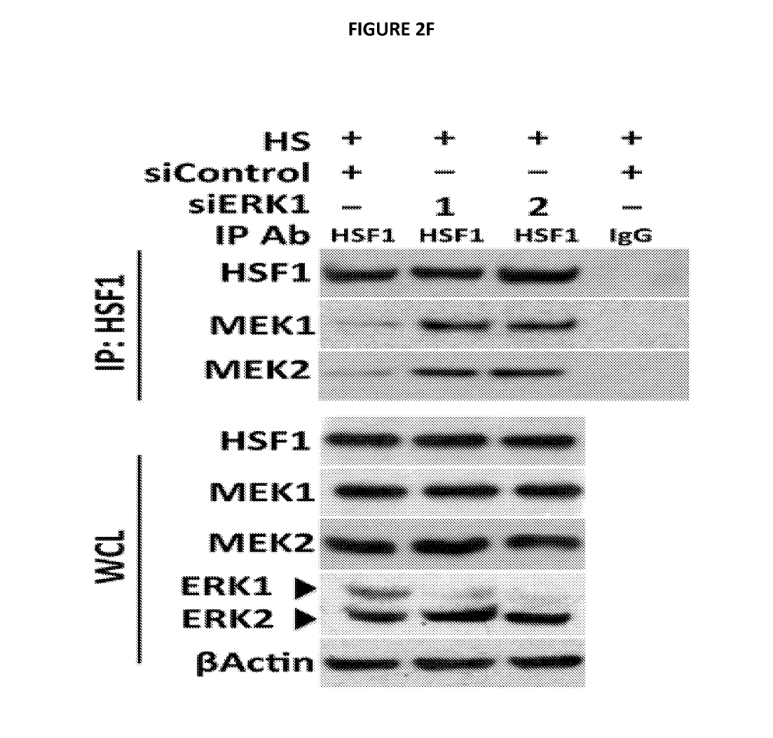

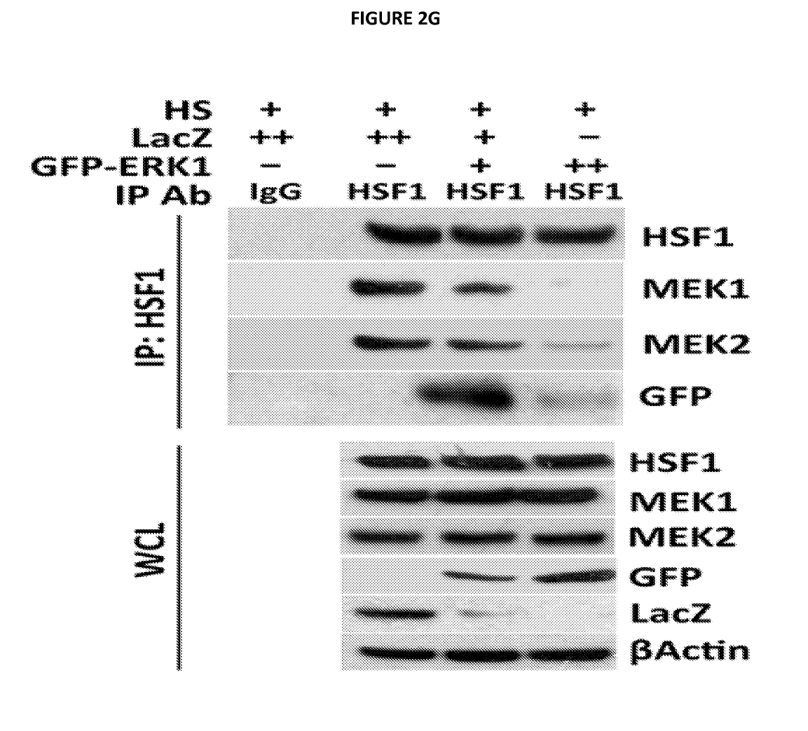

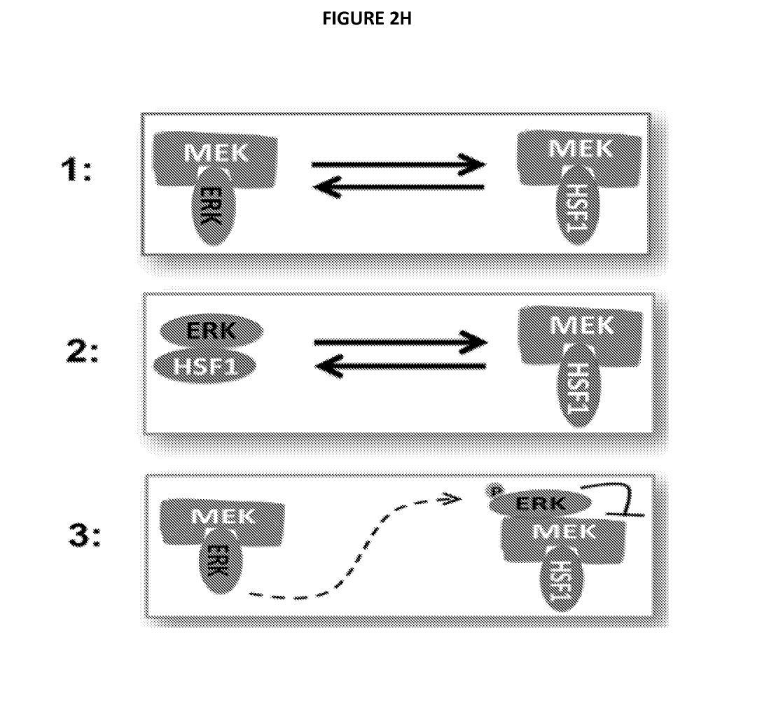

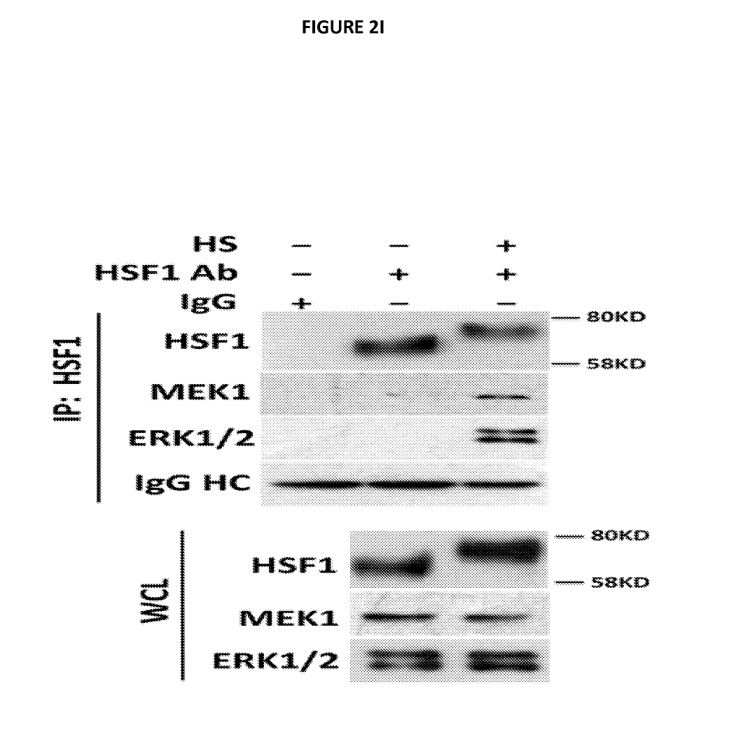

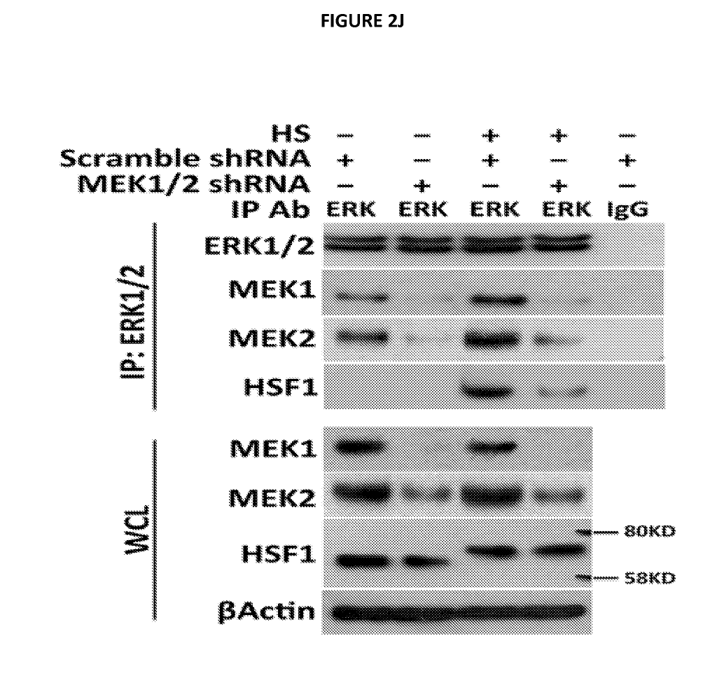

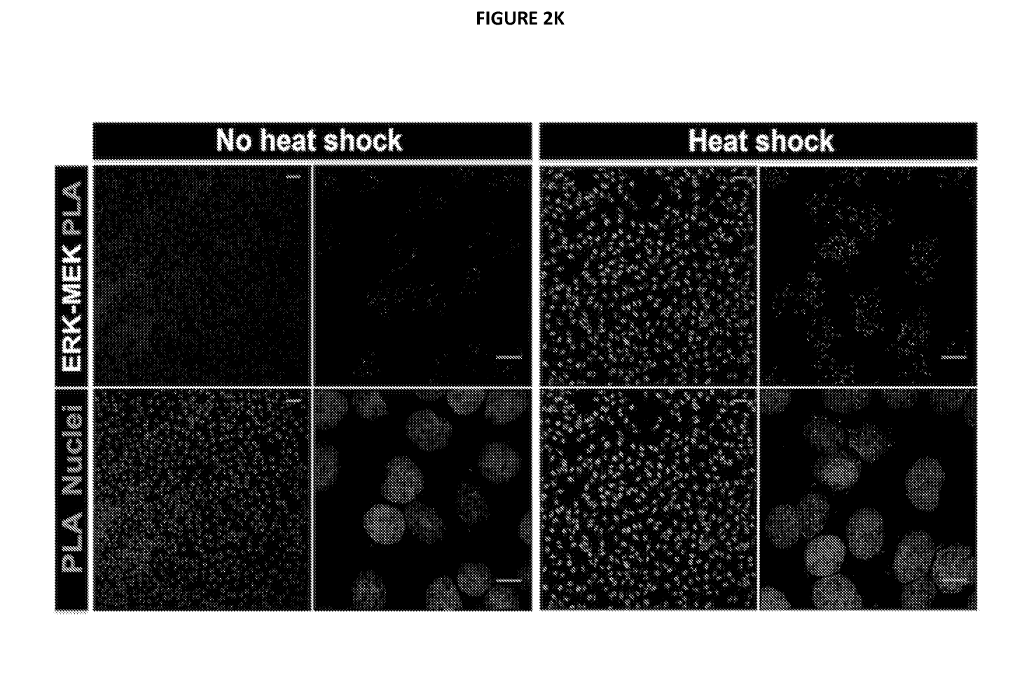

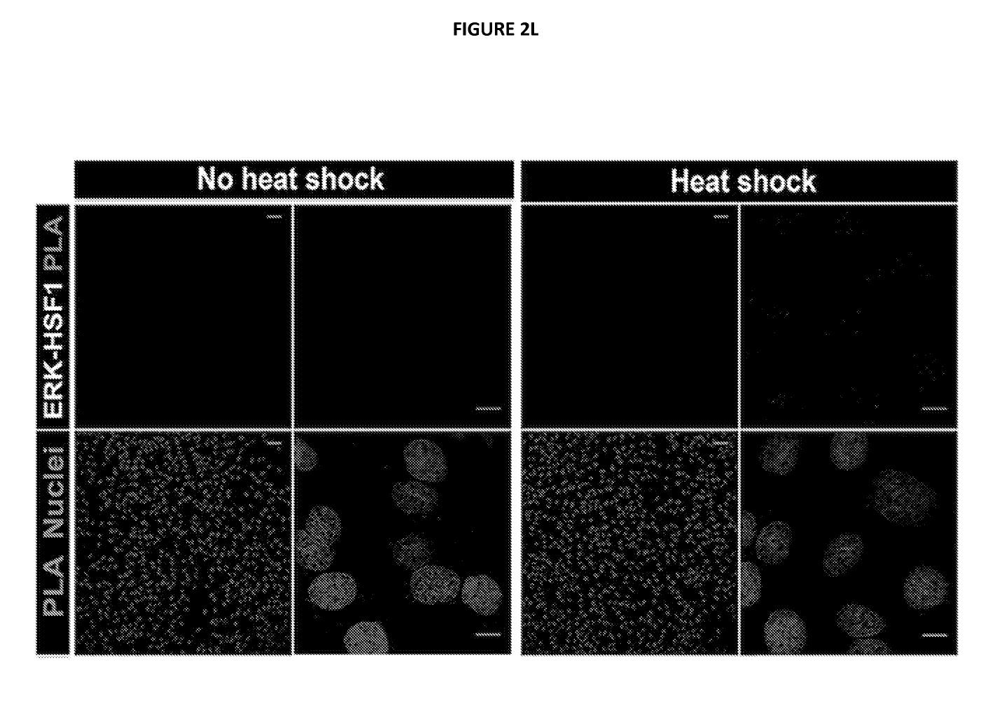

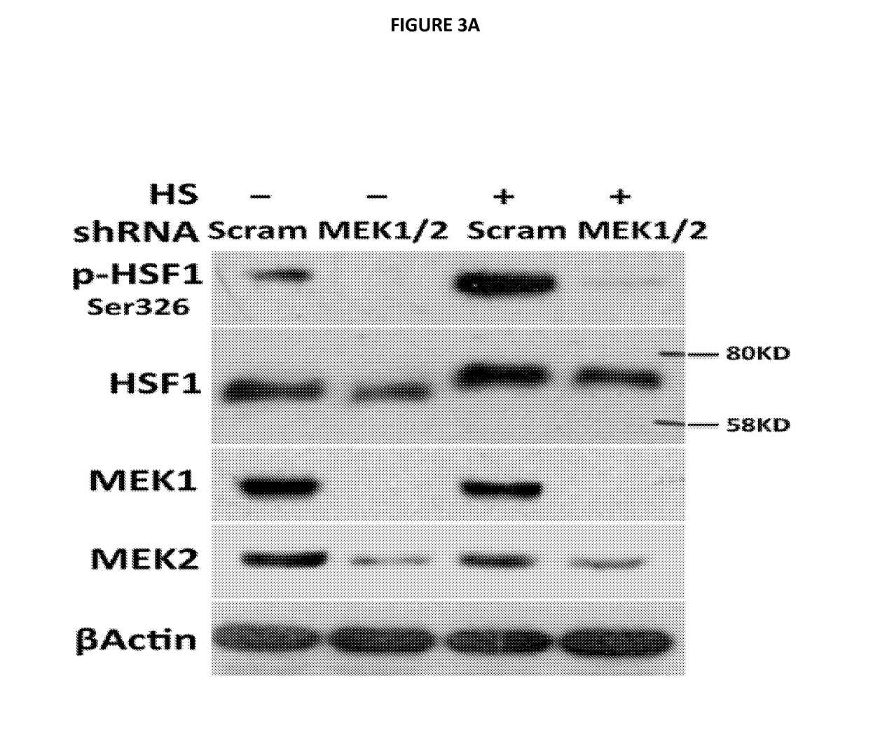

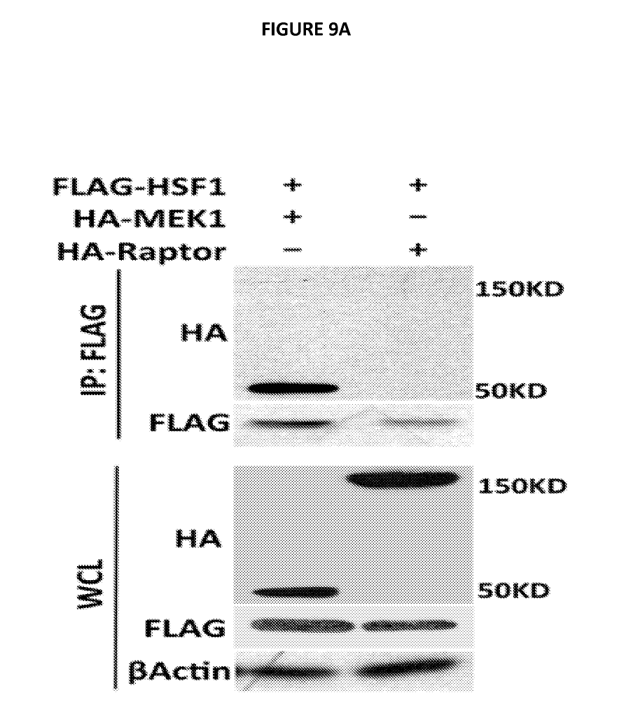

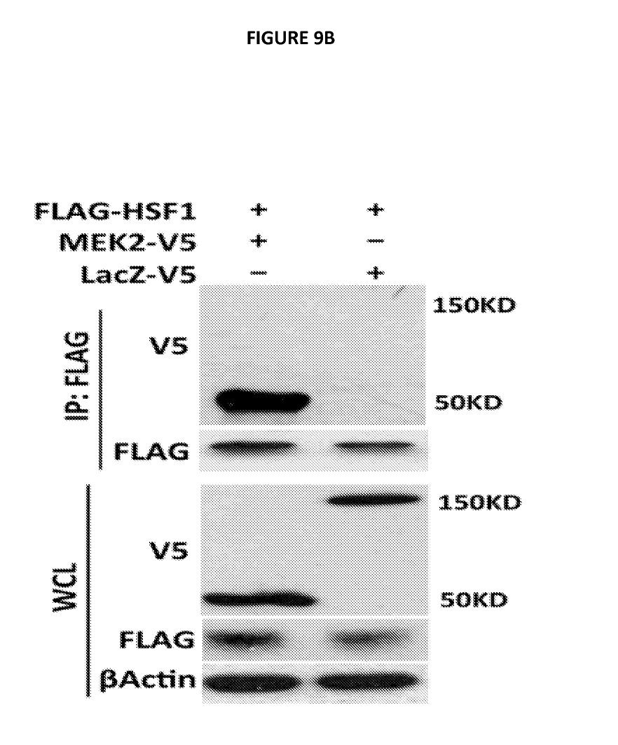

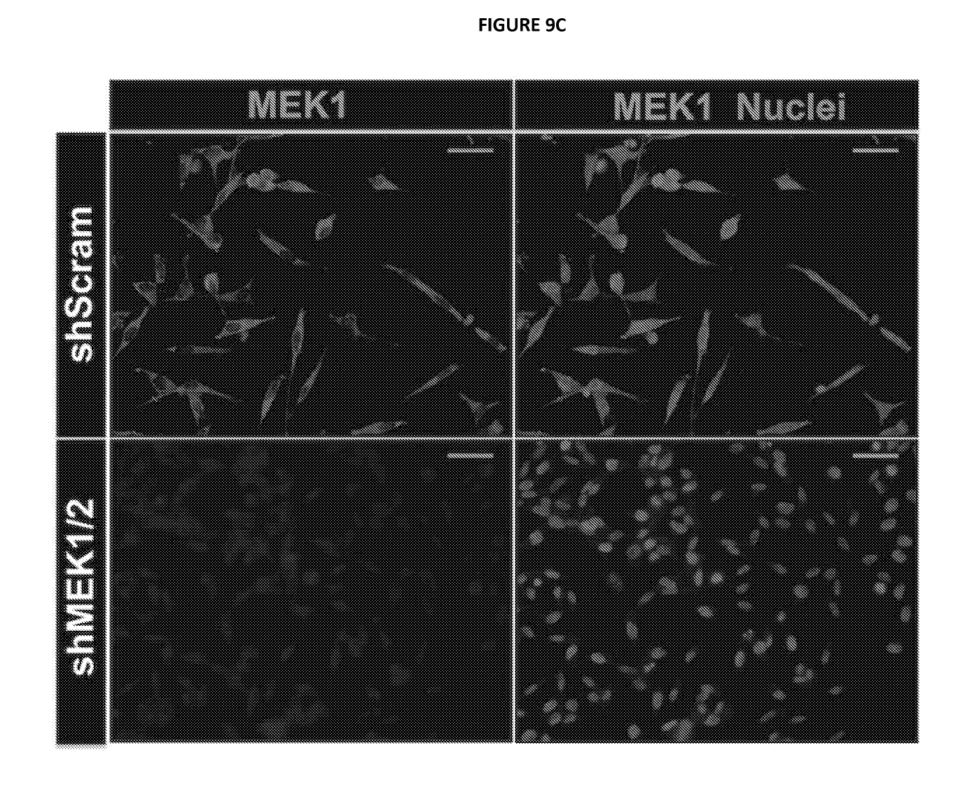

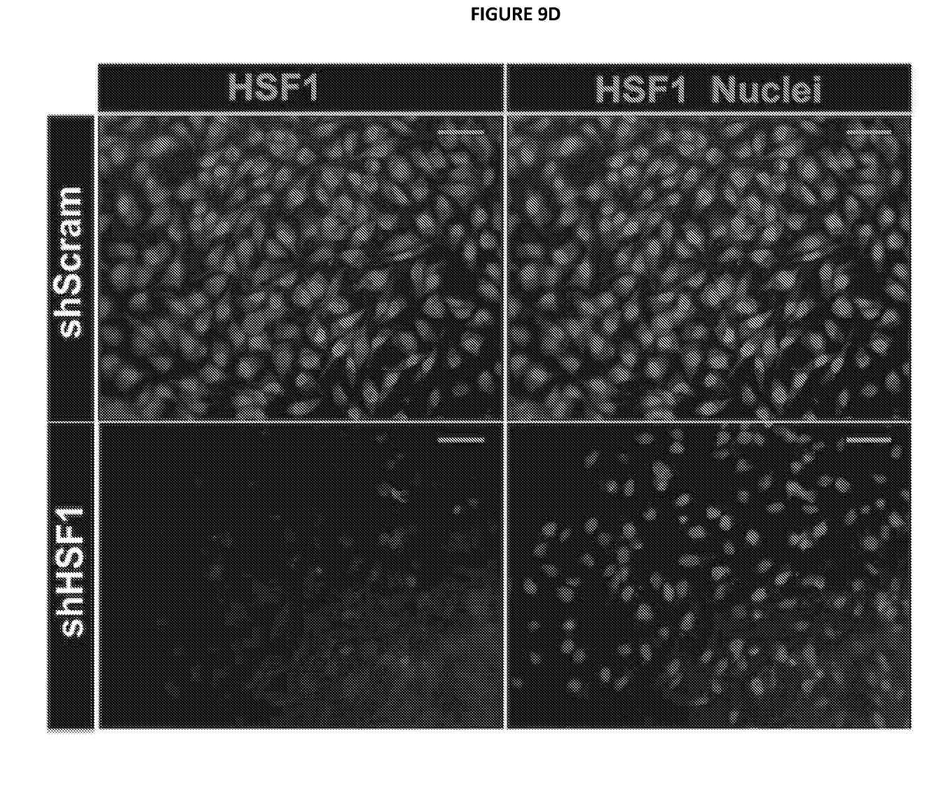

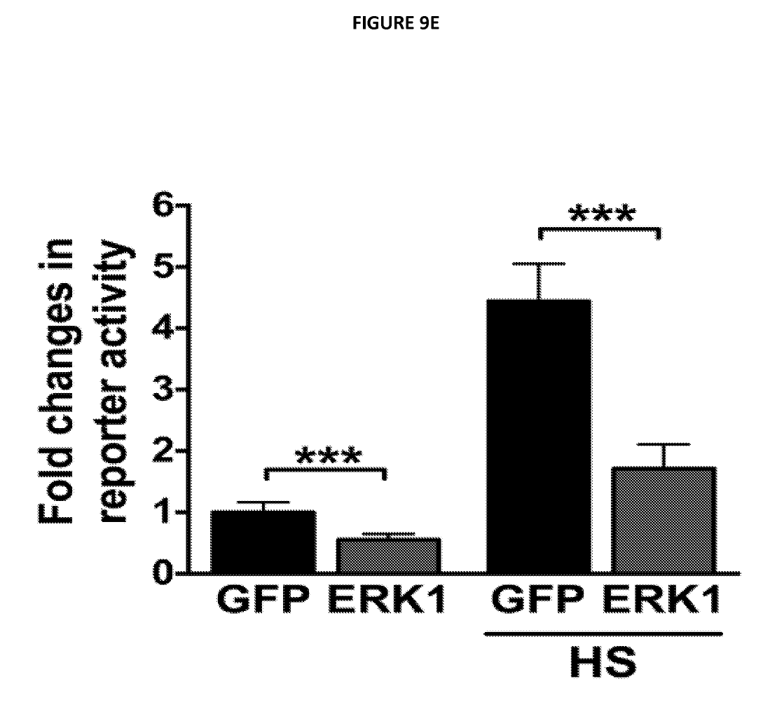

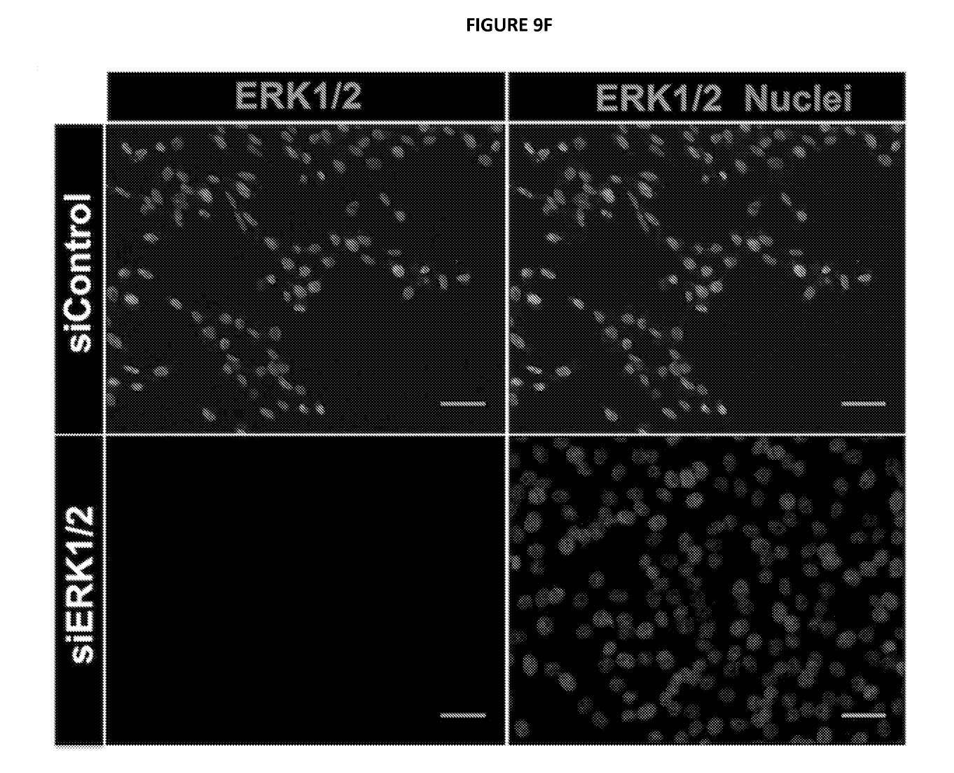

To determine whether MEK directly activates HSF1, we examined endogenous MEK-HSF1 interactions by co-immunoprecipitation (co-IP). While no evident MEK1/2 proteins were precipitated with HSF1 without HS, HS caused a marked co-IP (FIGS. 2A and 2B), showing a stress-inducible MEK-HSF1 interaction. The mobility shift of HSF1 marks HS-induced phosphorylation (FIG. 2A). These MEK-HSF1 interactions were verified via expression of recombinant proteins (FIGS. 9A and 9B). To determine whether MEK and HSF1 are in direct contact, we employed the Proximity Ligation Assay (PLA) technique (Clausson et al., 2011). Antibody specificities were validated by immunostaining (FIGS. 9C and 9D). In MEK-proficient cells, PLA signals were marginally visible without HS and HS intensified these signals (FIG. 2C). In MEK-deficient cells, only faint signals were detected even after HS (FIG. 2C), confirming the specificity of PLA. Of note, PLA signals were more manifest in the nucleus than in the cytoplasm (FIG. 2C), revealing a prominently nuclear localization of interactions. These results strongly suggest a direct MEK-HSF1 association.

MEK1 and MEK2 form either homo- or heterodimers in vivo (Catalanotti et al., 2009). To address which type of dimer binds HSF1, we examined MEK1-HSF1 interactions in the deficiency of MEK2. Under HS more MEK1 proteins were precipitated with HSF1 in MEK2-deficient cells (FIG. 2D). Similarly, MEK1 deficiency heightened MEK2-HSF1 interactions (FIG. 2E), revealing a competition between the two MEK isoforms for HSF1 binding and suggesting that MEK homodimers can interact with HSF1.

### Example 4. ERK Suppresses MEK-HSF1 Interactions to Inactivate HSF1

To elucidate how ERK inactivates HSF1, we first examined the impact of ERK on MEK-mediated HSF1 activation. Whereas ERK1 depletion promoted MEK-HSF1 interactions (FIG. 2F), ERK1 overexpression mitigated these interactions and suppressed HSF1 (FIGS. 2G and 9E). Thus, we contemplated three possible scenarios (FIG. 2H): 1) both MEK substrates, ERK and HSF1, compete for MEK interaction; 2) ERK, like MEK, binds HSF1 and thereby competes for HSF1 interaction; and 3) ERK inhibits MEK kinase activity towards HSF1. Each of the first two scenarios predicts competition between two protein complexes; in contrast, the third scenario predicts that ERK assembles with MEK and HSF1 into a single protein complex. Interestingly, under HS HSF1 precipitated both MEK and ERK (FIG. 2I), and ERK precipitated both MEK and HSF1 (FIG. 2J). Although these results do not exclude the existence of independent MEK-ERK and MEK-HSF1 complexes, they argue against the two complexes being stable and prevalent, as depicted in the first scenario. To test the second scenario, we detected ERK-HSF1 interactions by PLA, as this scenario predicts HSF1 as a substrate for both ERK and MEK. The specificity of ERK antibodies was validated in ERK-depleted cells (FIG. 9F). In contrast to evident MEK1-ERK interactions (FIG. 2K), no apparent PLA signals denoting ERK-HSF1 interactions were detected (FIG. 2L), suggesting lack of direct contact between these two proteins. Moreover, while ERK1 overexpression mitigated MEK-HSF1 interactions, less ERK1 proteins were precipitated with HSF1 (FIG. 2G), conflicting with heightened ERK1-HSF1 interactions predicted by the second scenario. Thus, these results not only refute the second scenario but also suggest that ERK complexes with HSF1 via MEK, in line with the third scenario. Importantly, MEK depletion markedly diminished ERK-HSF1 co-IP (FIG. 2J). These results suggest existence of a protein complex comprising ERK, MEK, and HSF1, wherein ERK suppresses HSF1 indirectly, via inhibition of MEK.

### Example 5. MEK Phosphorylates Ser326 to Activate HSF1

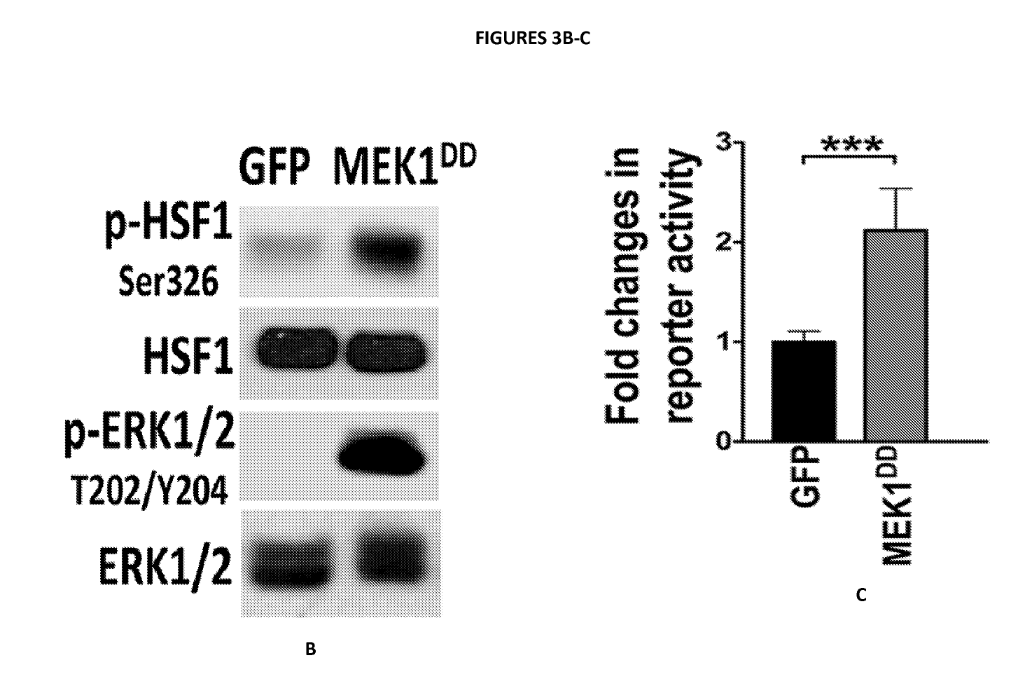

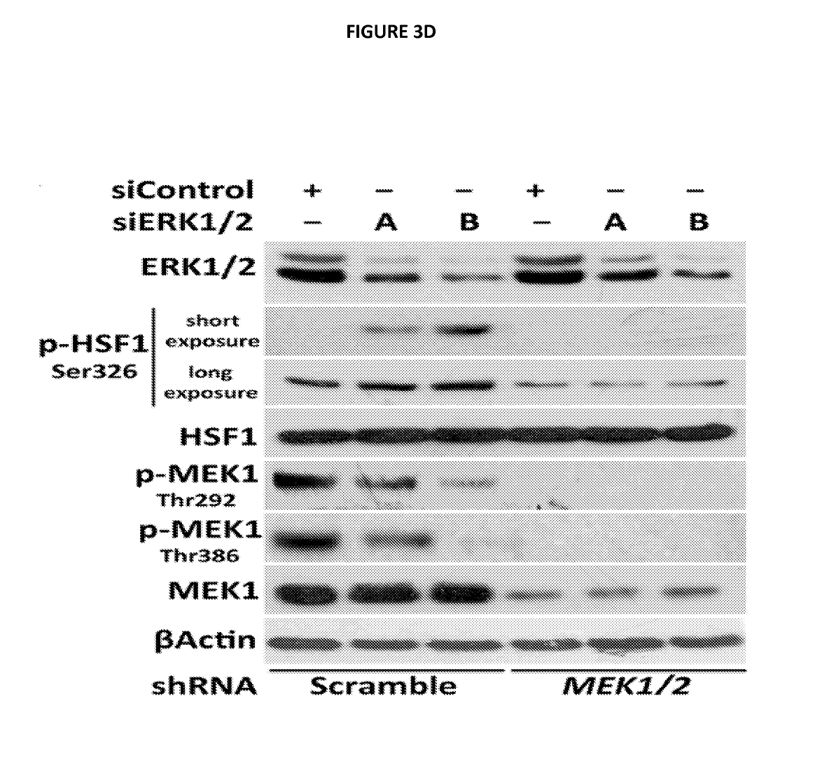

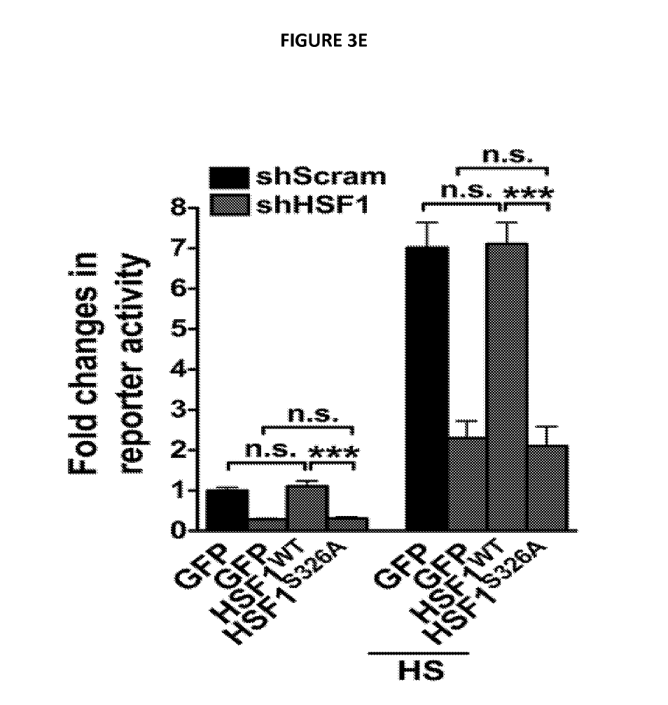

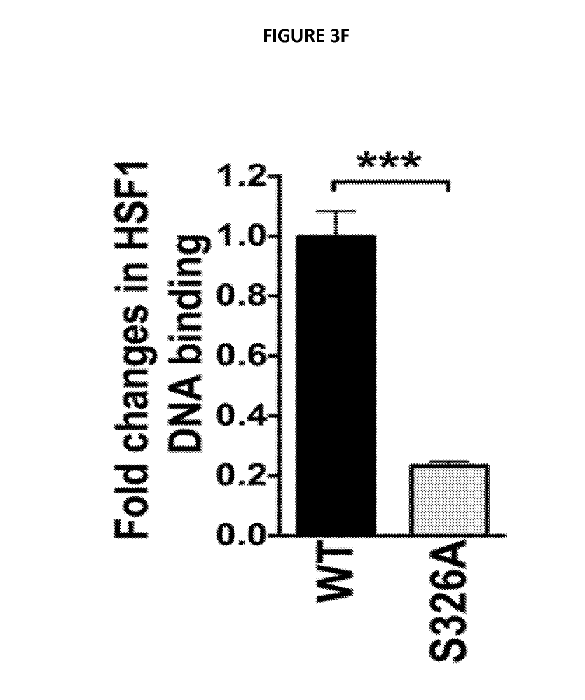

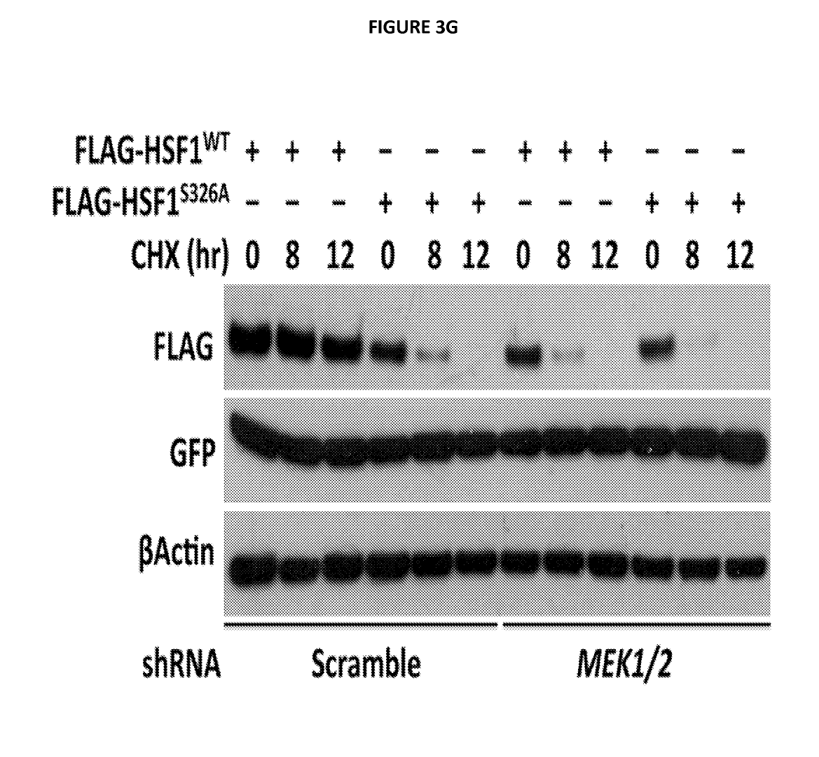

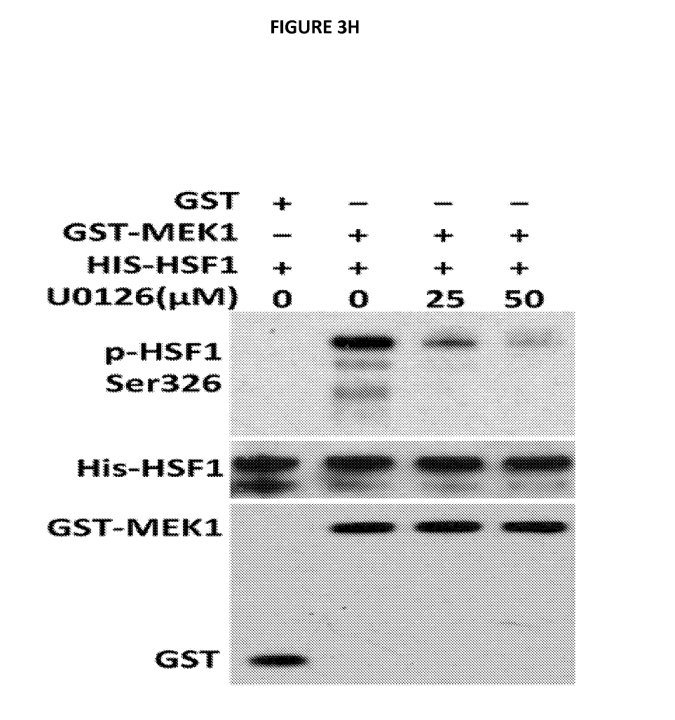

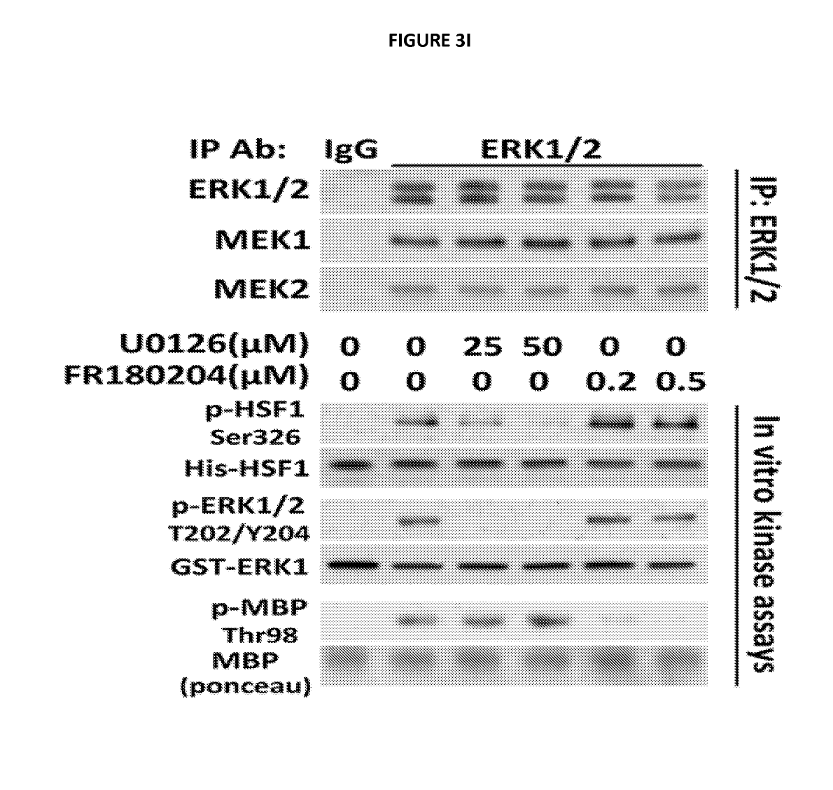

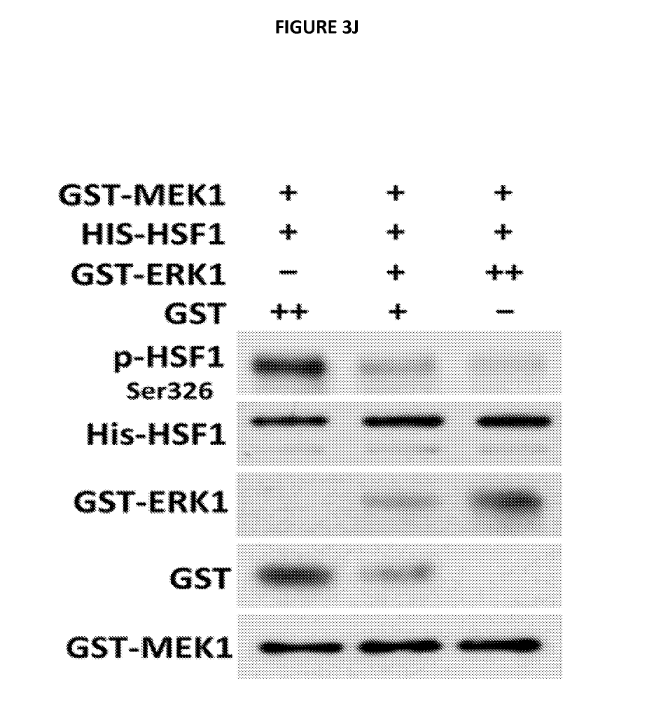

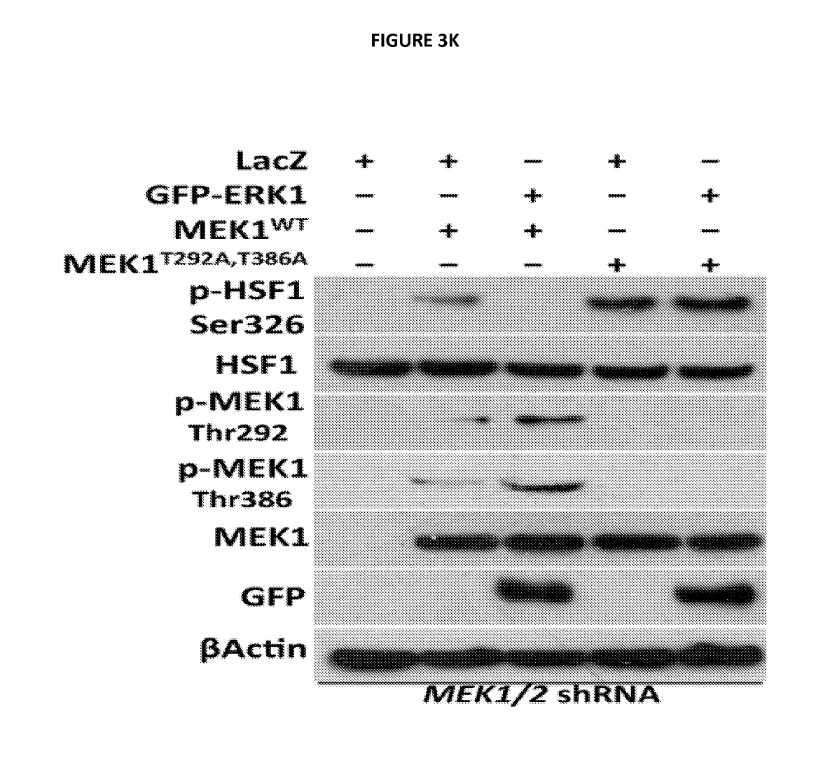

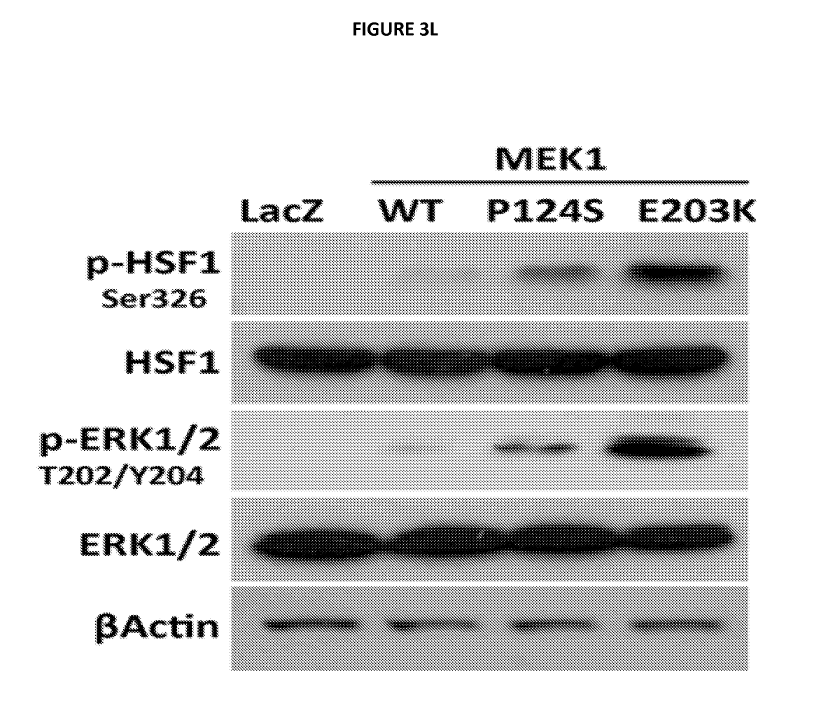

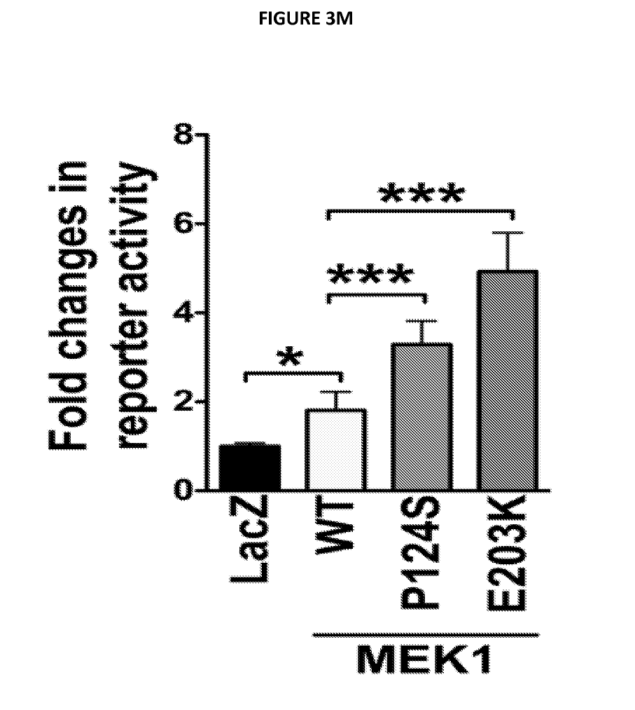

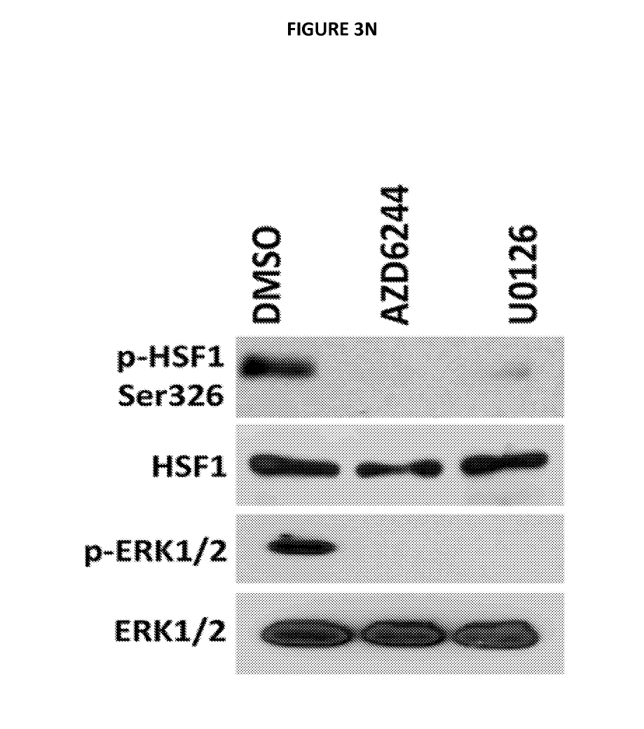

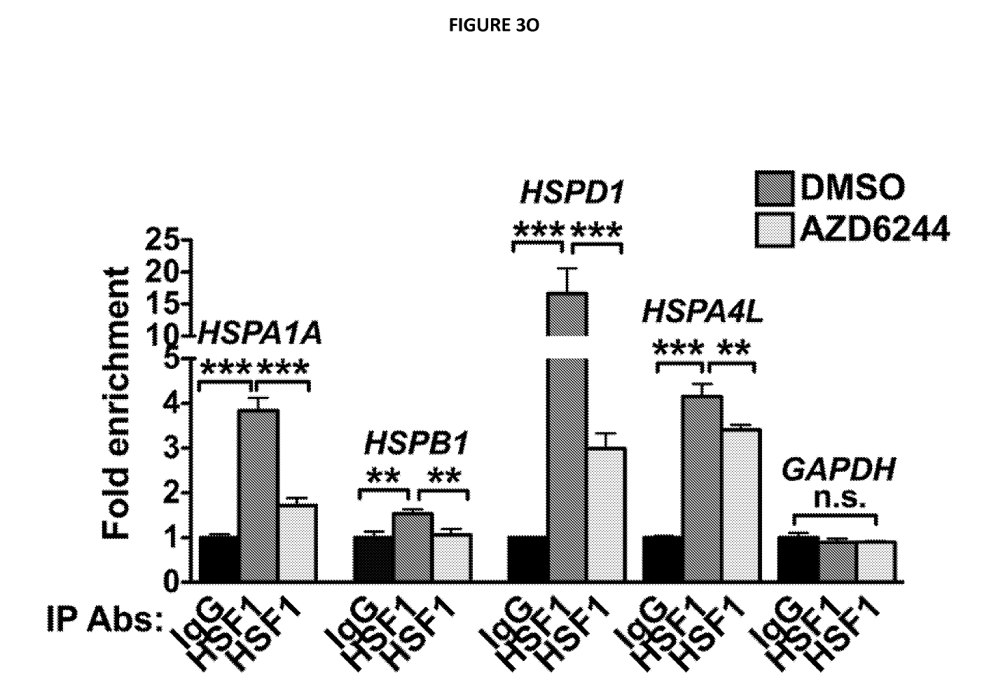

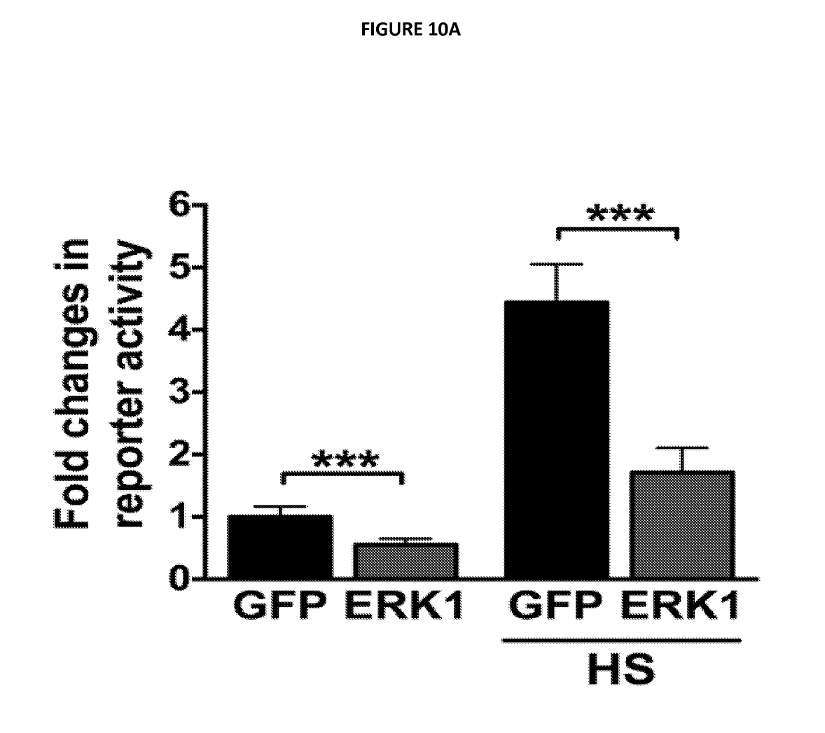

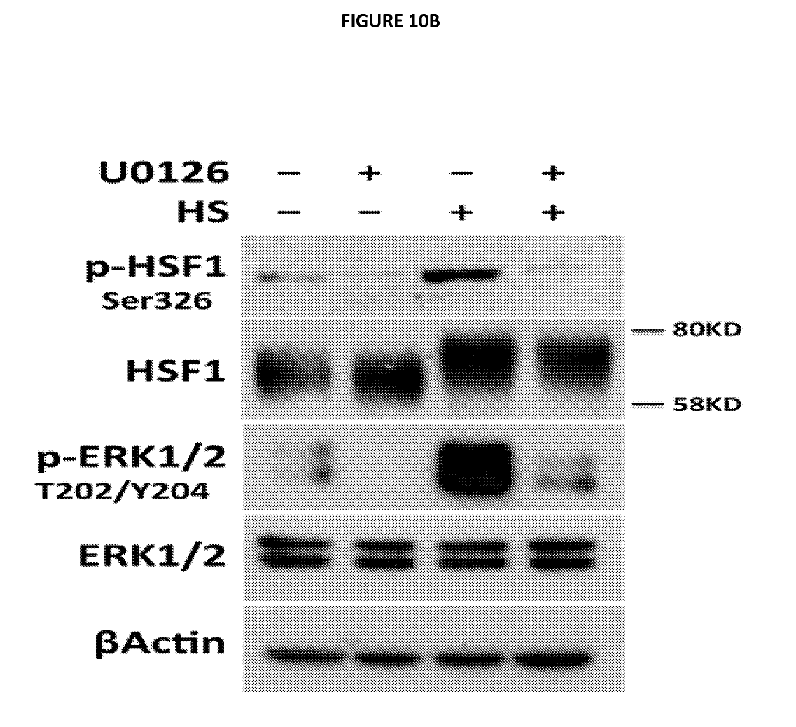

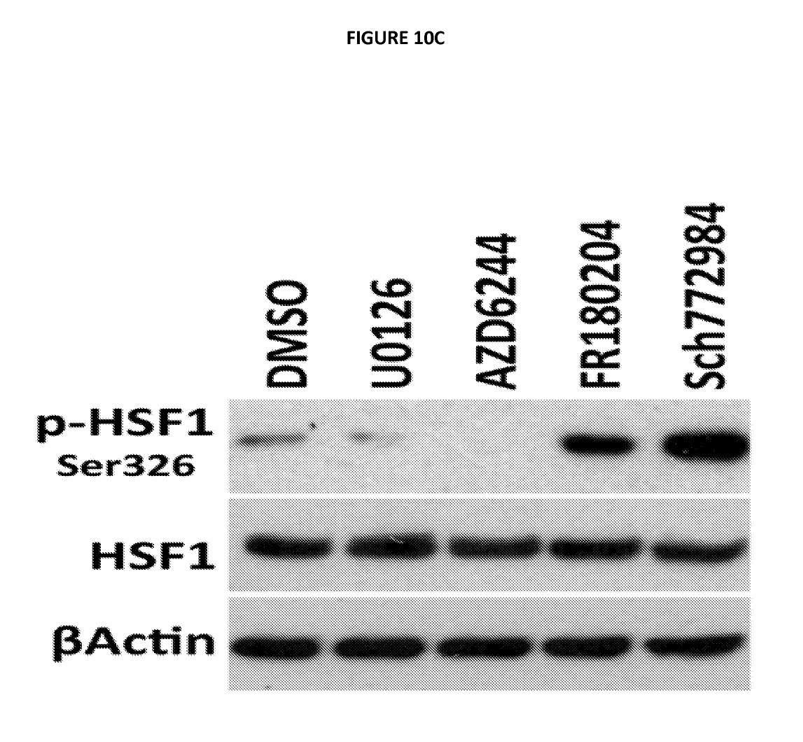

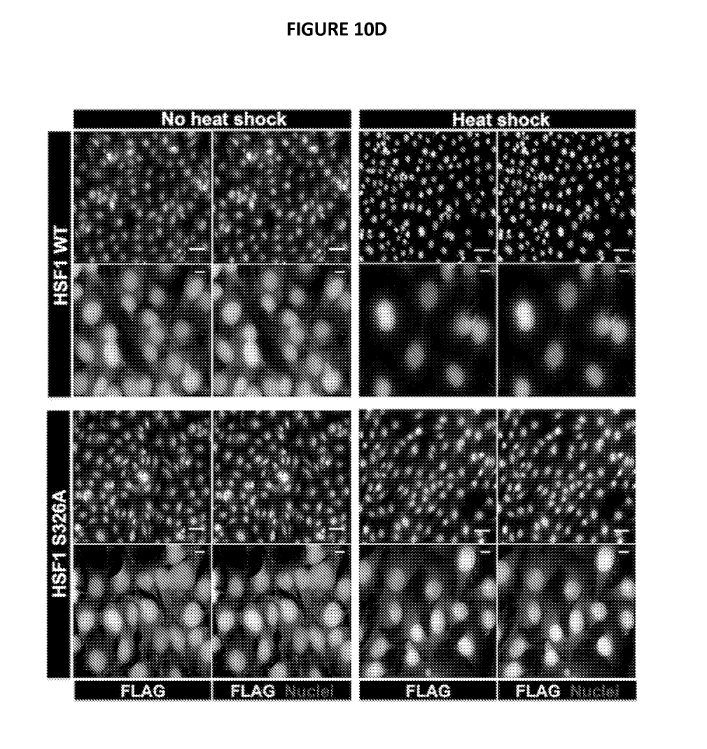

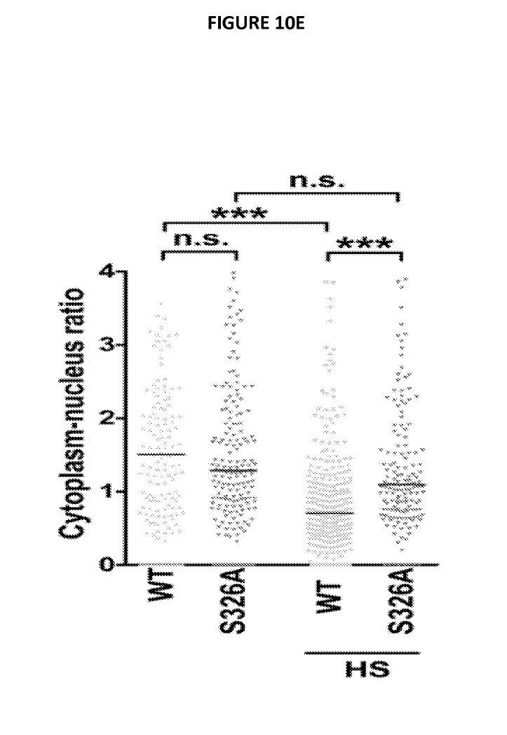

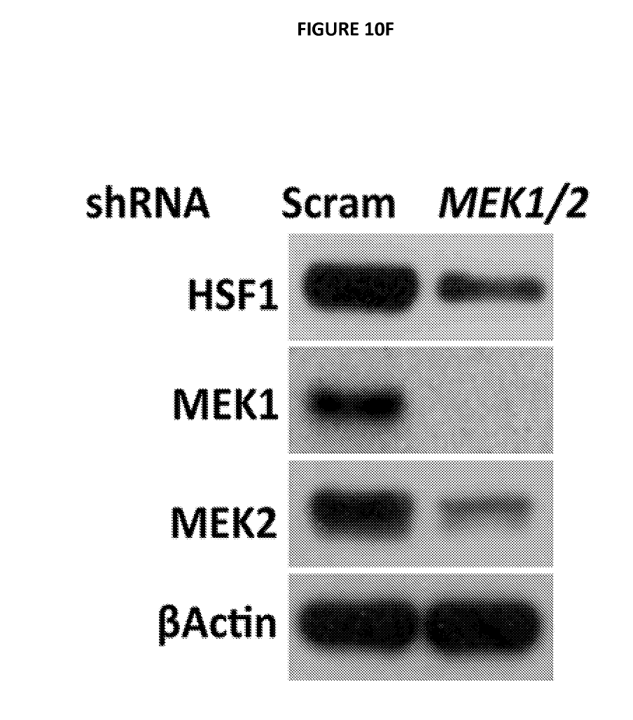

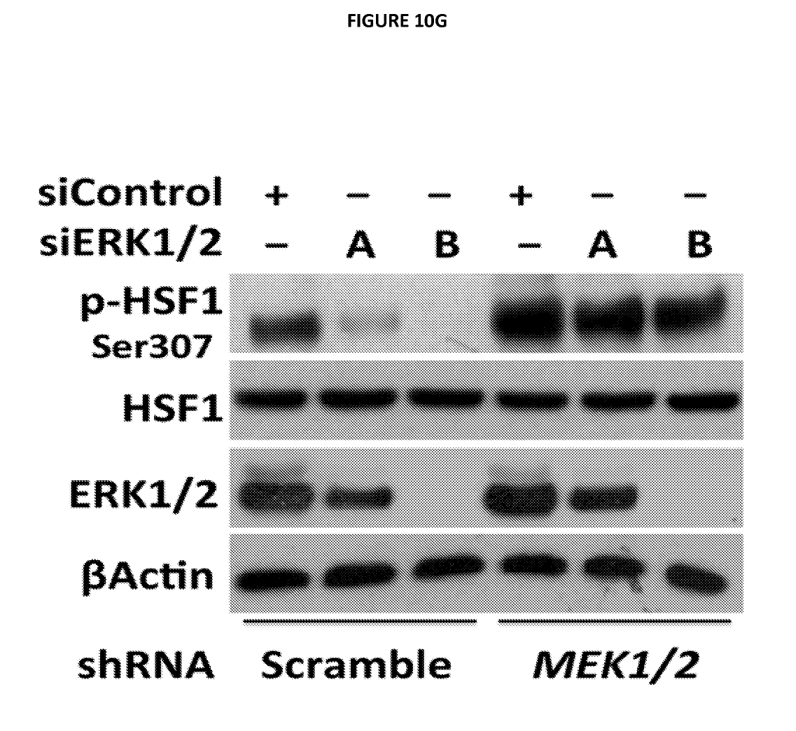

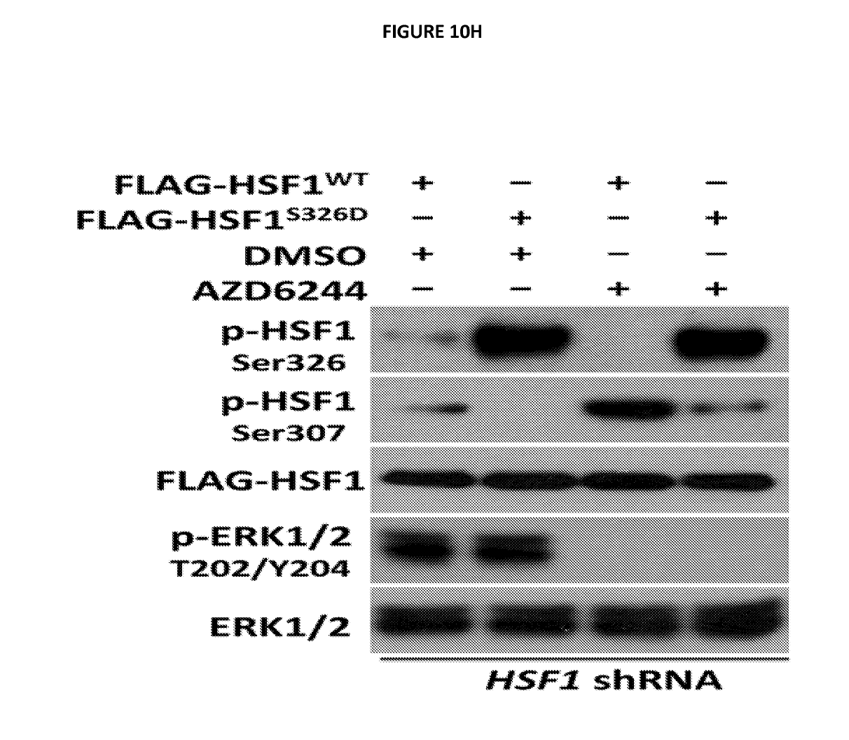

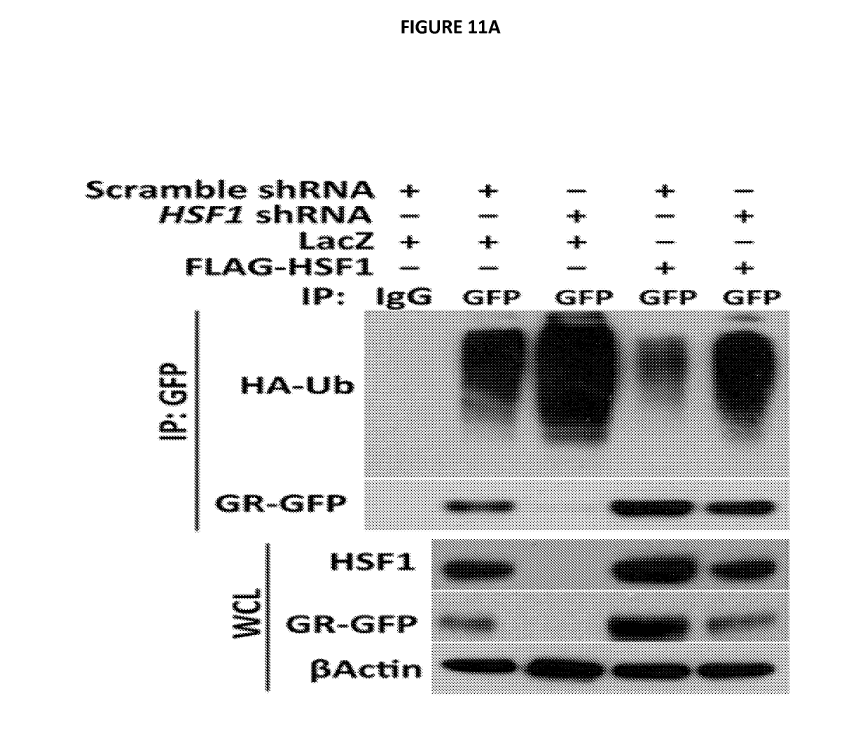

Under HS HSF1 undergoes a series of phosphorylating events, among which Ser326 phosphorylation stimulates its activation (Guettouche et al., 2005). Yet, the identity of the kinase remains elusive. To determine whether MEK phosphorylates Ser326, we examined the effect of MEK blockade on this modification using a phosphospecific antibody that recognized HSF1^{WT}, but not HSF1^{S326A}, proteins (FIG. 10A). Either MEK knockdown or U0126 treatment impaired Ser326 phosphorylation (FIGS. 3A and 10B). Conversely, a constitutively active mutant, MEK1^{DD} (S218D/S222D) (Brunet et al., 1994a), induced Ser326 phosphorylation and activation of HSF1 without HS (FIGS. 3B and 3C). ERK inhibition enhanced Ser326 phosphorylation; and, MEK depletion abolished this effect (FIGS. 3D and 10C), indicating MEK-dependent regulation. HSF1^{S326A} mutants displayed impaired transcription activities (FIG. 3E), congruent with their defective nuclear translocation and DNA-binding capacity (FIGS. 10D-E and 3F). Moreover, HSF1 proteins were reduced in MEK-deficient cells (FIGS. 3A and 10F). To determine whether MEK impacts HSF1 stability, we performed cycloheximide chase experiments. MEK depletion shortened the half-life of HSF1^{WT} protein; importantly, while HSF1^{S326A} mutants were less stable in MEK-proficient cells, their stability was not evidently affected by MEK deficiency (FIG. 3G), suggesting that MEK stabilizes HSF1 largely via Ser326 phosphorylation. These results indicate that MEK controls in vivo HSF1 Ser326 phosphorylation, a modification critical for its activation and stability.

In vitro, recombinant MEK1 proteins directly phosphorylated HSF1 at Ser326; and U0126 blocked this event (FIG. 3H). ERK was reported to phosphorylate HSF1 at Ser307 (Chu et al., 1998), implying a direct ERK-HSF1 interaction. To exclude direct Ser326 phosphorylation by ERK, we performed in vitro HSF1 phosphorylation using immunoprecipitated endogenous ERK complexes that would comprise ERK associated with or without MEK. Although precipitated complexes phosphorylated Ser326, this event was blocked by U0126, but not by FR180204 (FIG. 3I). U0126, but not FR180204, blocked phosphorylation of recombinant ERK1 proteins by the same precipitates (FIG. 3I). In contrast, FR180204, but not U0126, blocked phosphorylation of myelin basic protein (MBP), a known ERK substrate (Ahn et al., 1991), by the same precipitates (FIG. 3I), showing ERK blockade by FR180204. These results strongly suggest that MEK, rather than ERK, directly phosphorylates Ser326. Moreover, recombinant ERK1 proteins impeded in vitro HSF1 Ser326 phosphorylation by recombinant MEK1 proteins (FIG. 3J), consistent with the suppression of Ser326 phosphorylation by ERK in vivo. In contrast, ERK promotes HSF1 Ser307 phosphorylation. ERK depletion diminished Ser307 phosphorylation; however, this effect was largely abolished in MEK-deficient cells (FIG. 10G), again indicating MEK dependence. MEK inhibition impaired Ser326 phosphorylation but enhanced Ser307 phosphorylation of HSF1^{WT} proteins (FIG. 10H). Interestingly, phosphomimetic mutant HSF1^{S326D} proteins displayed reduced basal Ser307 phosphorylation and resisted induction of this phosphorylation by MEK inhibition (FIG. 10H). These results support that Ser326 phosphorylation by MEK represses Ser307 phosphorylation, and that ERK impacts HSF1 Ser326 and Ser307 phosphorylation via MEK inhibition. Activated ERK phosphorylates Thr292/386 to inhibit MEK1 (Brunet et al., 1994b). MEK1^{T292A,T386A} mutants both heightened basal HSF1 Ser326 phosphorylation and blocked ERK-mediated suppression of this phosphorylation in cells depleted of endogenous MEK (FIG. 3K), indicating that ERK suppresses Ser326 phosphorylation via feedback phosphorylation of MEK.

Interestingly, two MEK1 mutations identified in human melanomas, P124S and E203K (Nikolaev et al., 2012), caused constitutive HSF1 phosphorylation and activation (FIGS. 3L and 3M). Conversely, in human melanoma cells MEK inhibitors impaired constitutive HSF1 phosphorylation and binding to HSP promoters (FIGS. 3N and 3O). These results indicate that MEK controls both inducible HSF1 activation in stressed cells and constitutive HSF1 activation in malignant cells.

### Example 6. MEK Preserves Cellular Proteostasis

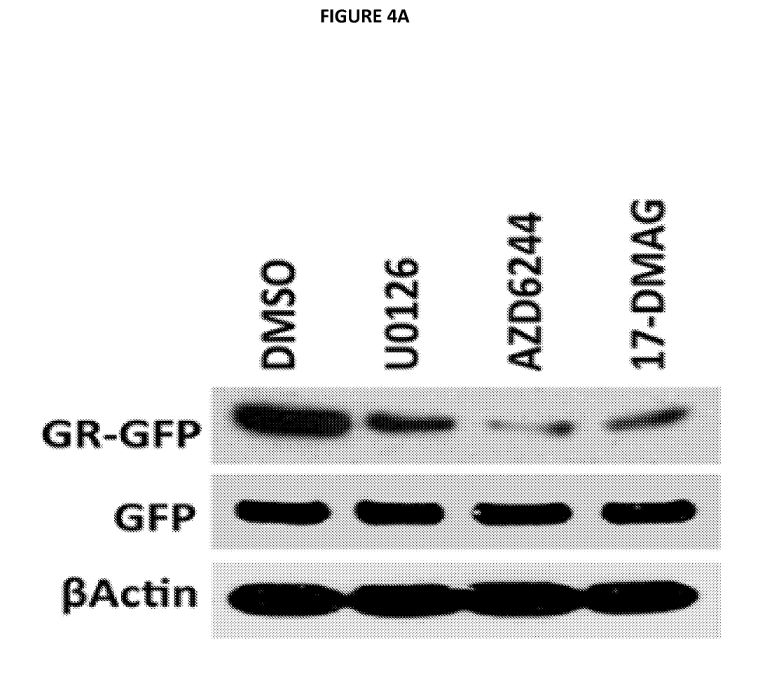

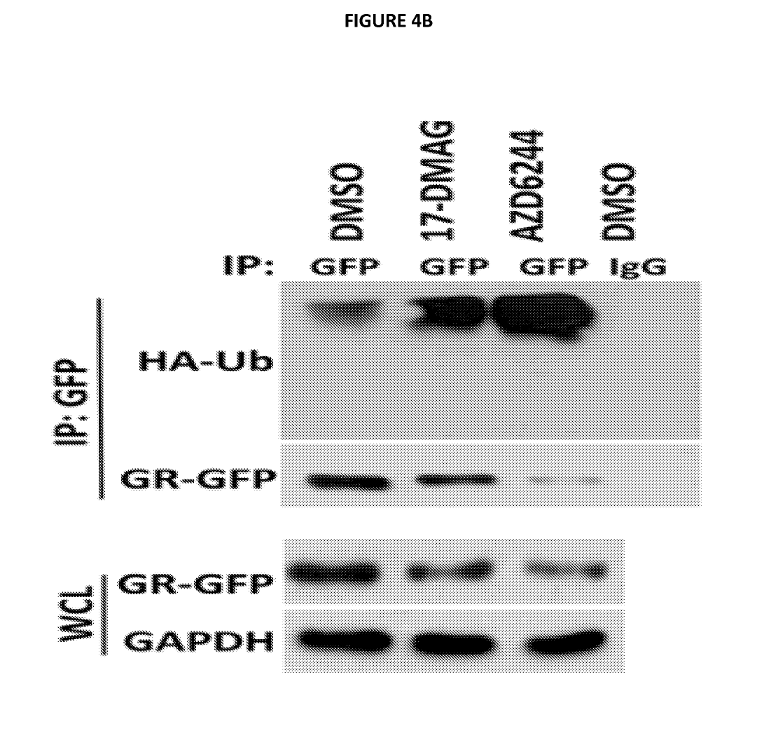

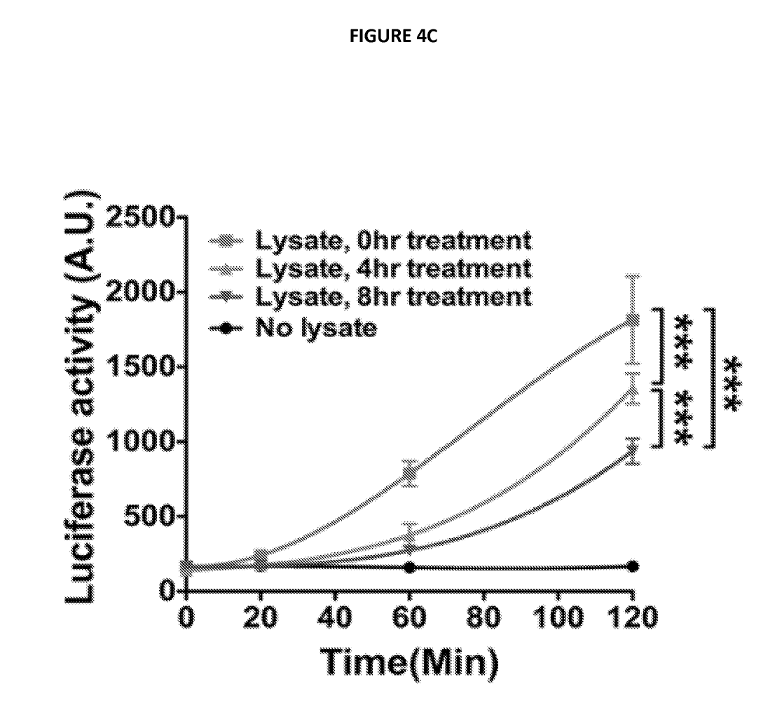

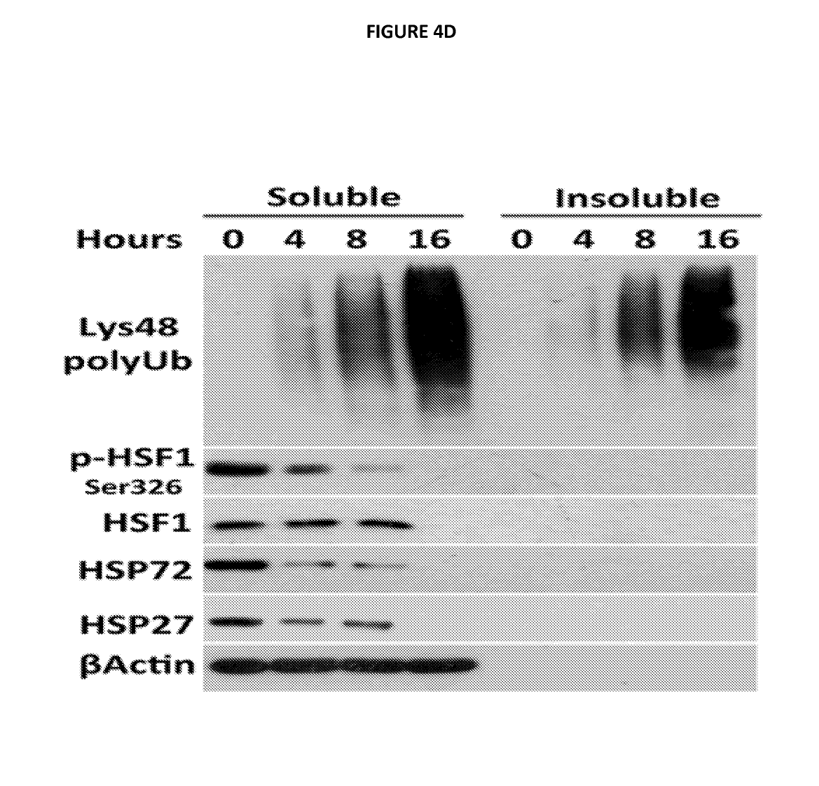

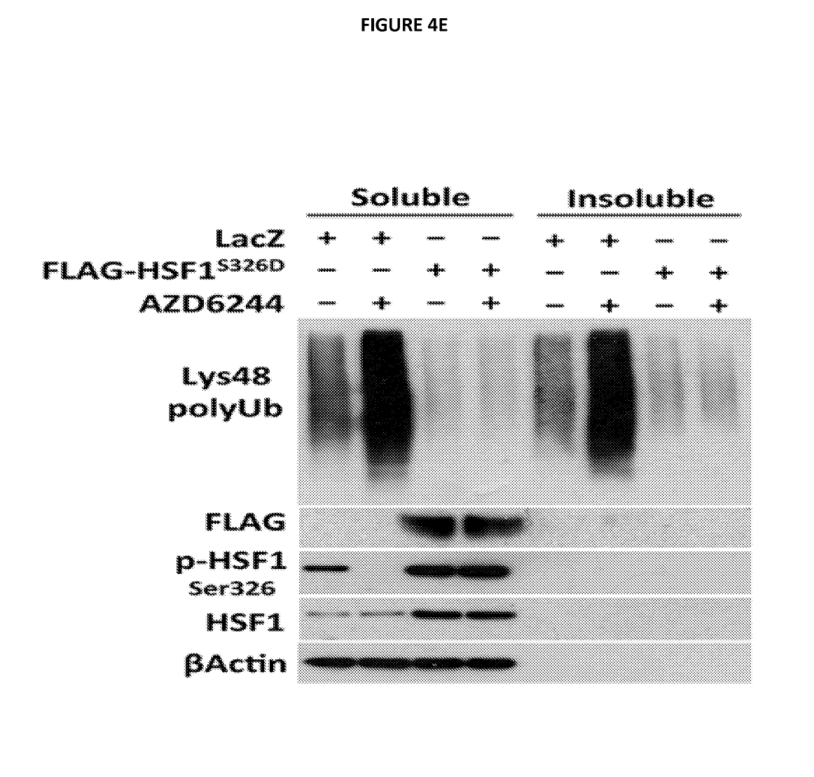

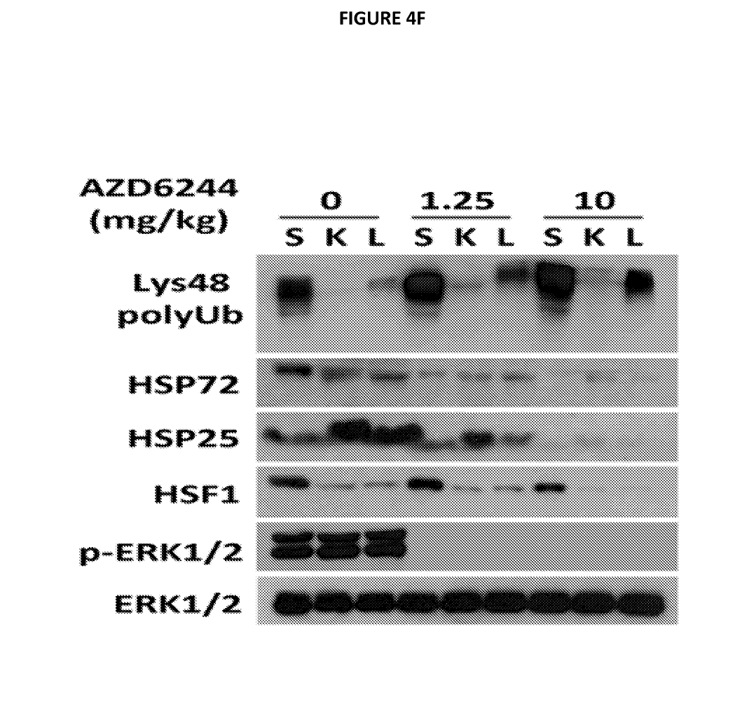

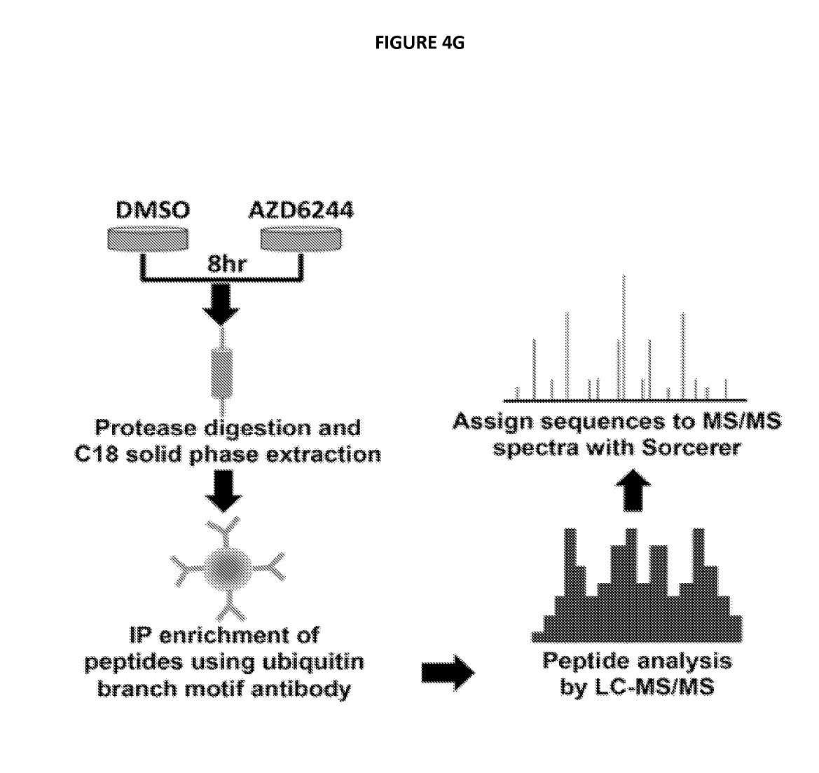

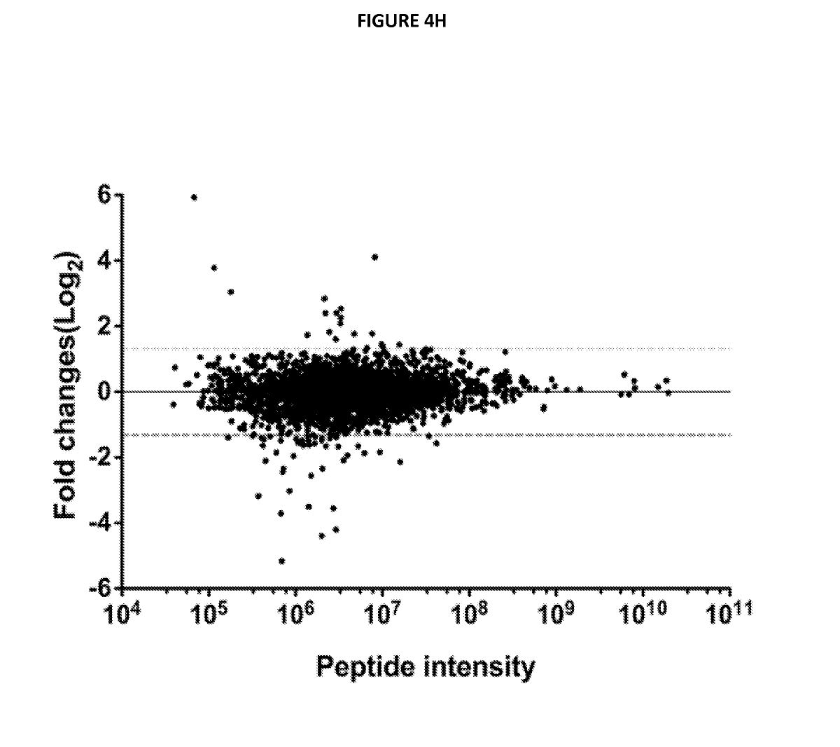

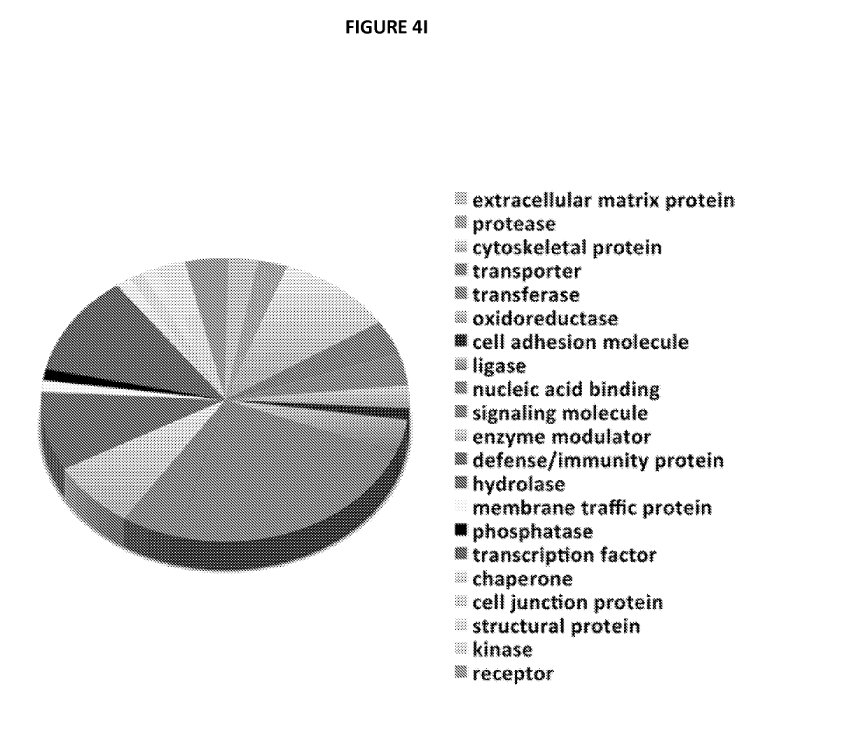

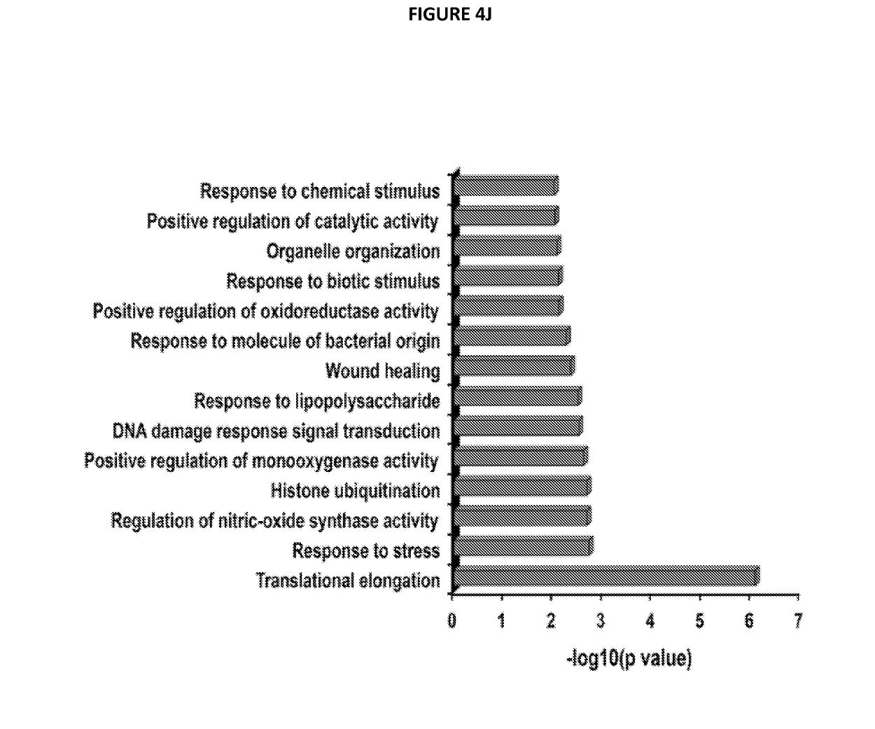

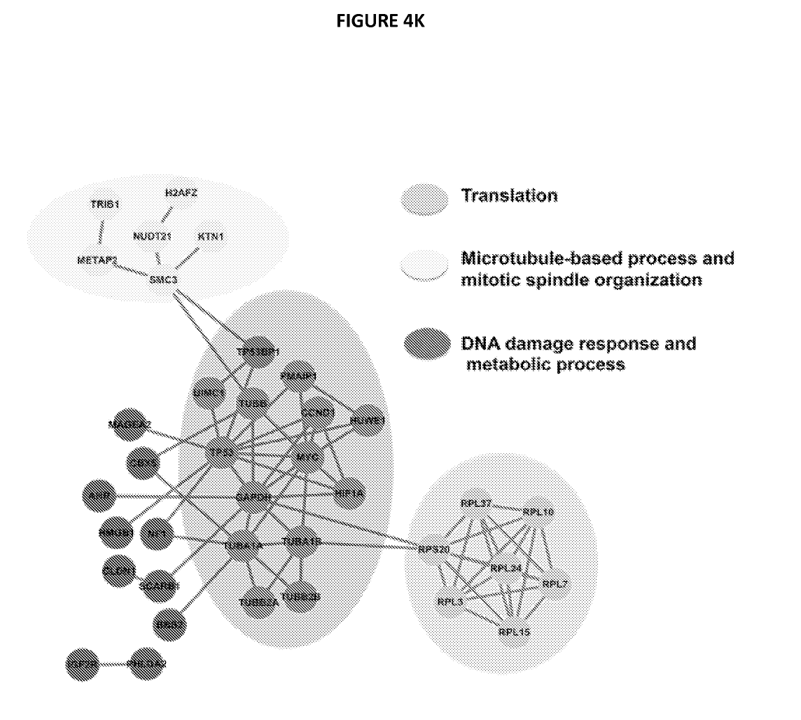

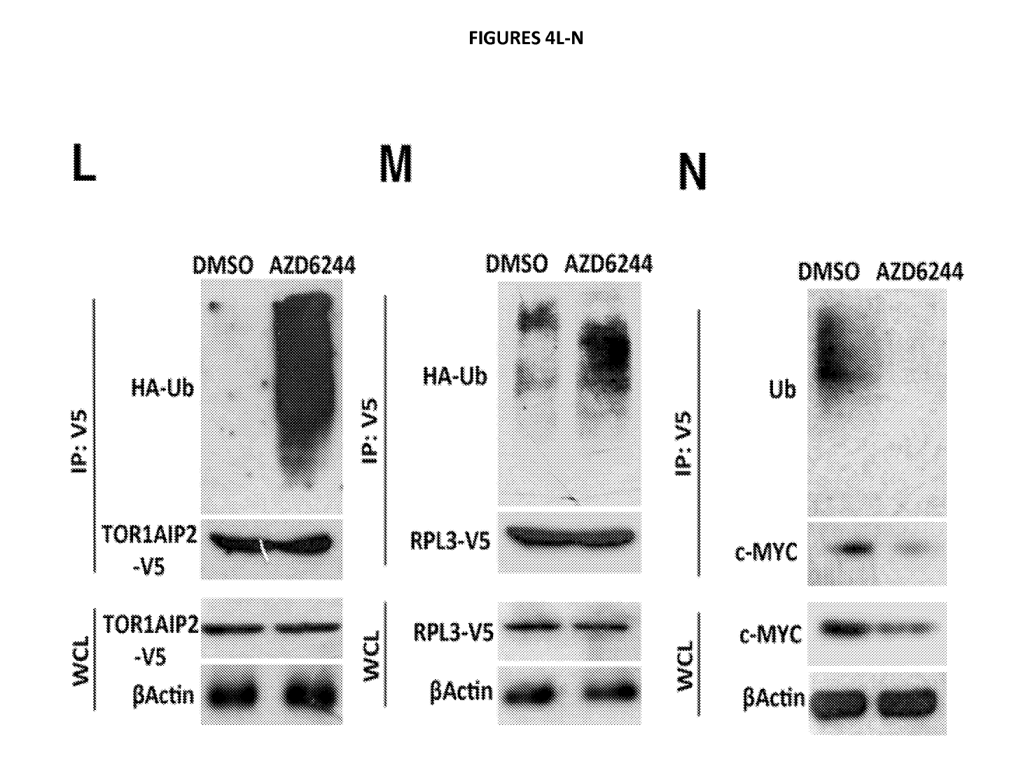

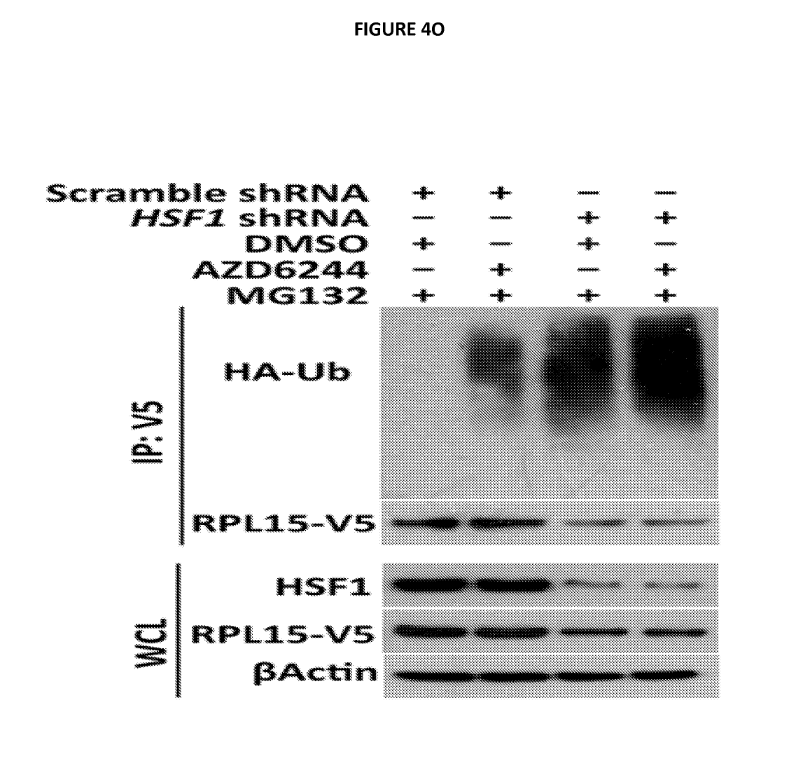

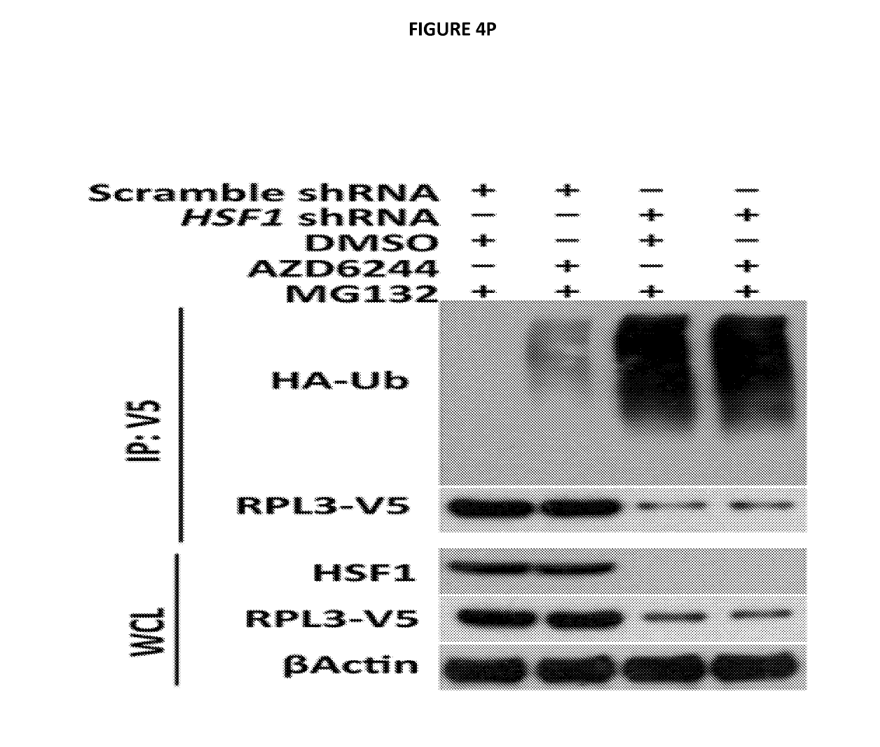

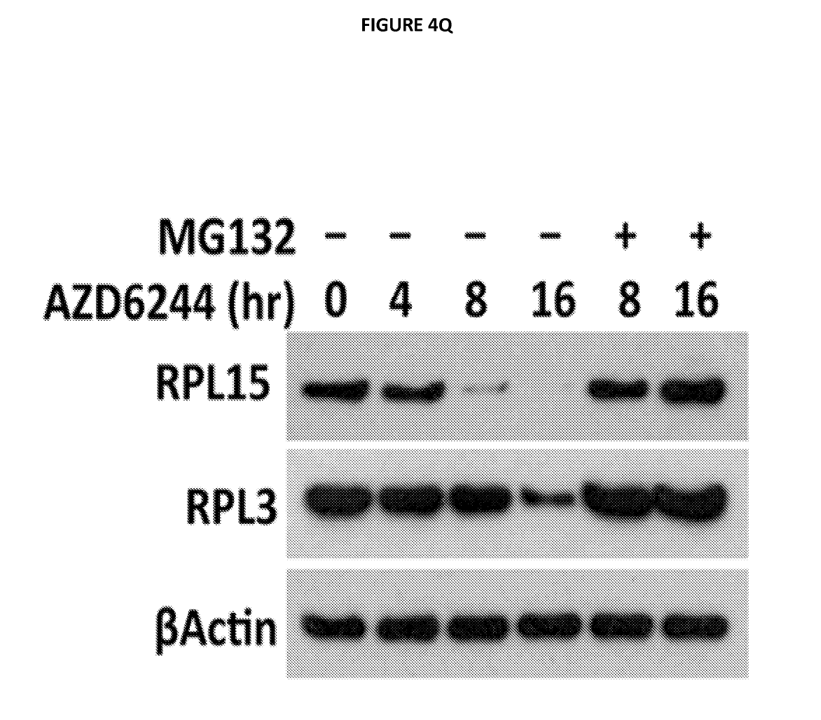

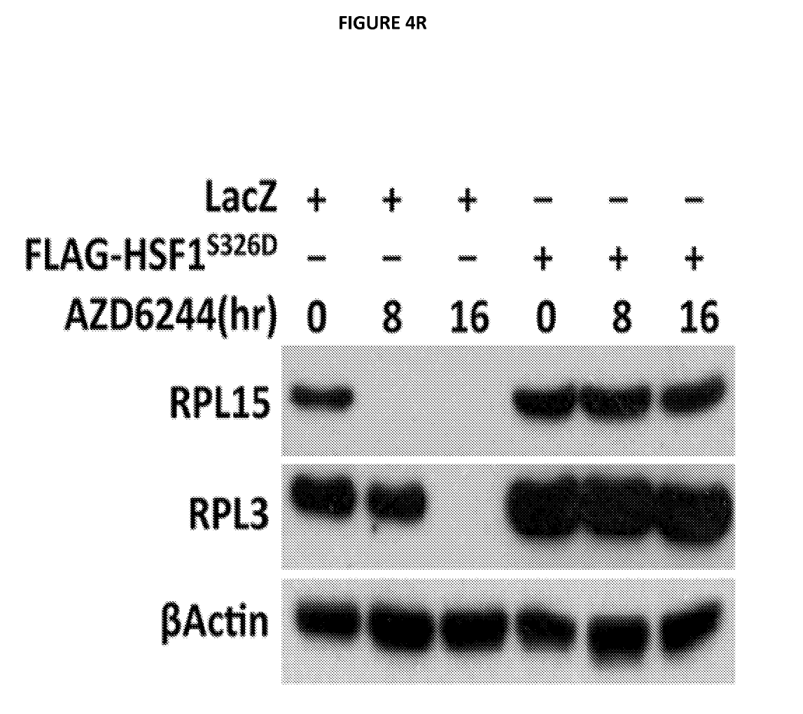

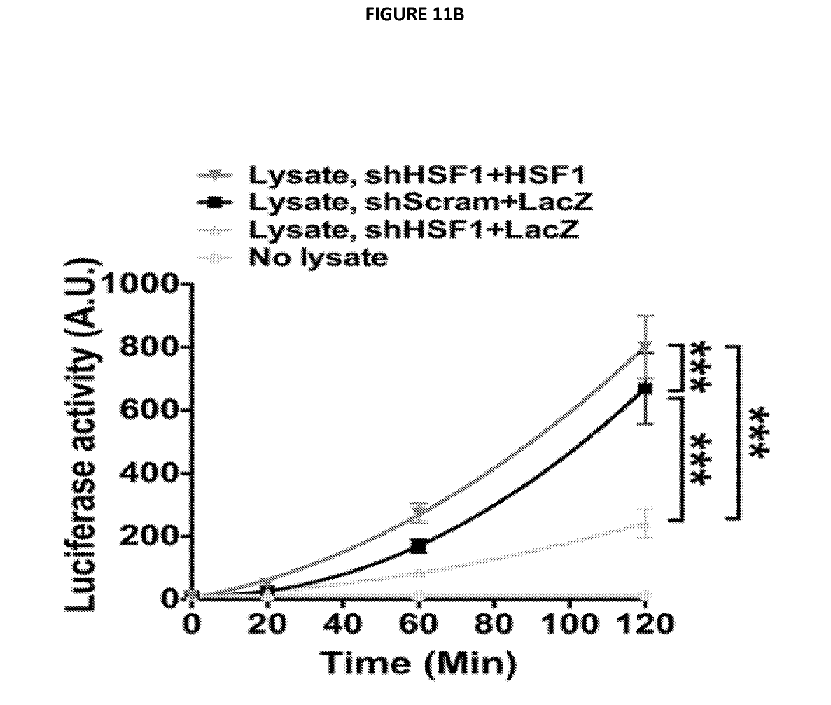

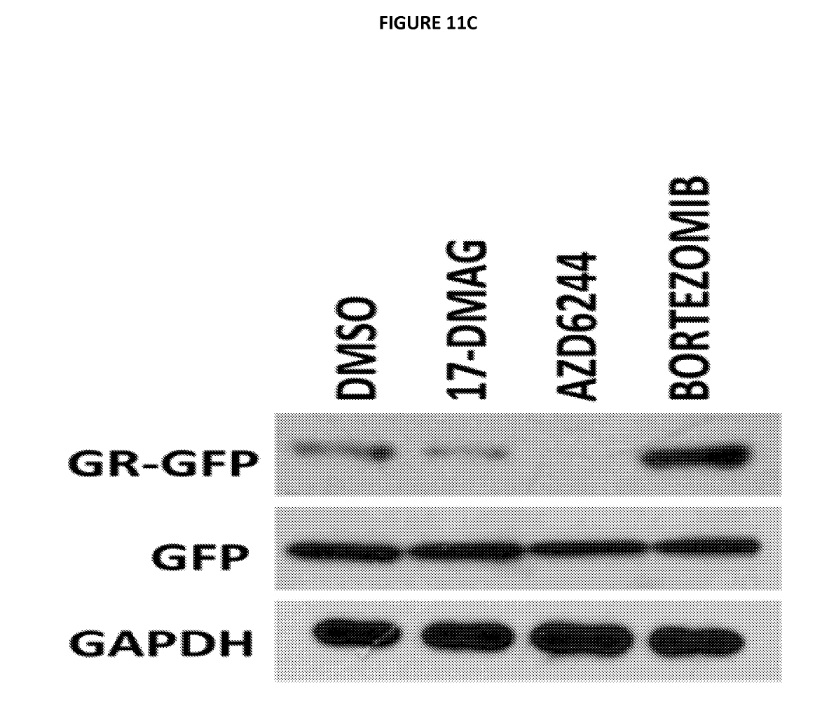

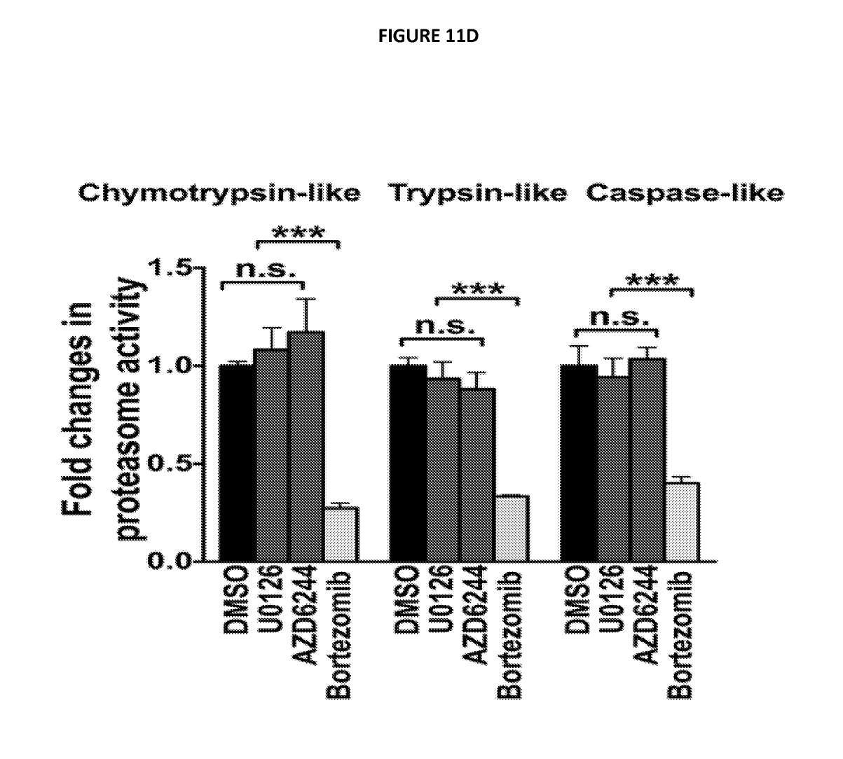

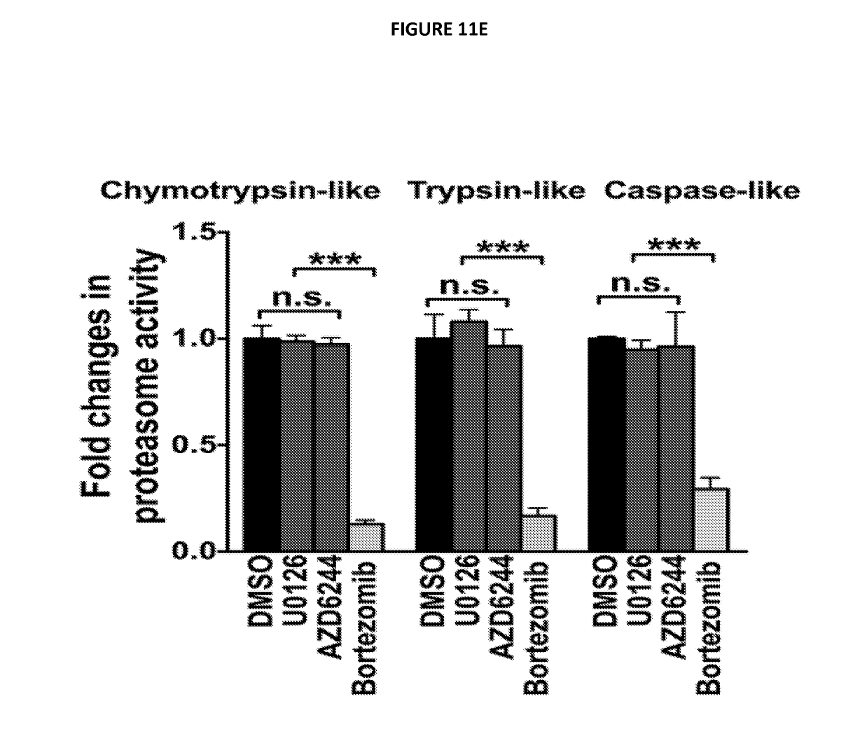

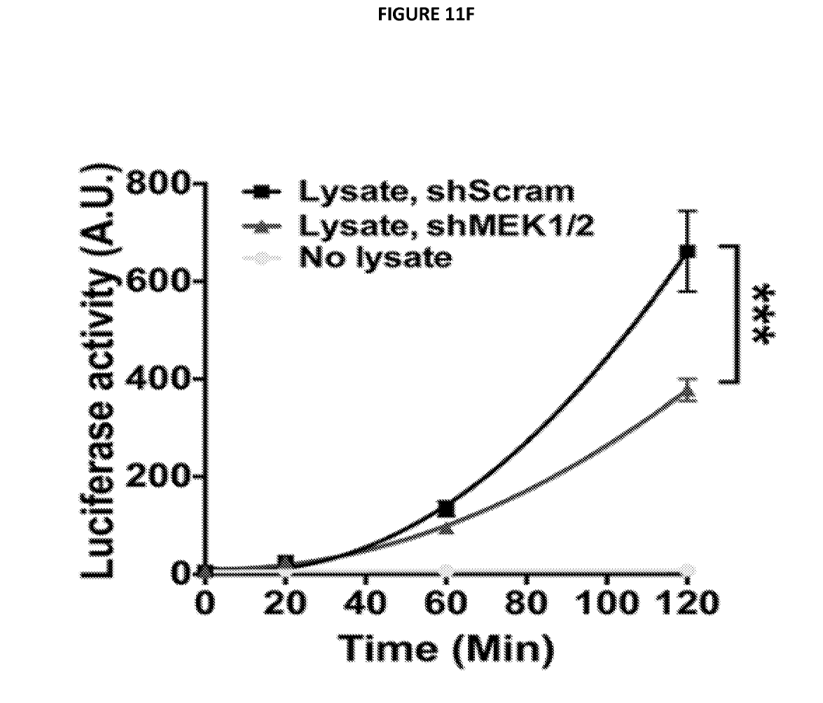

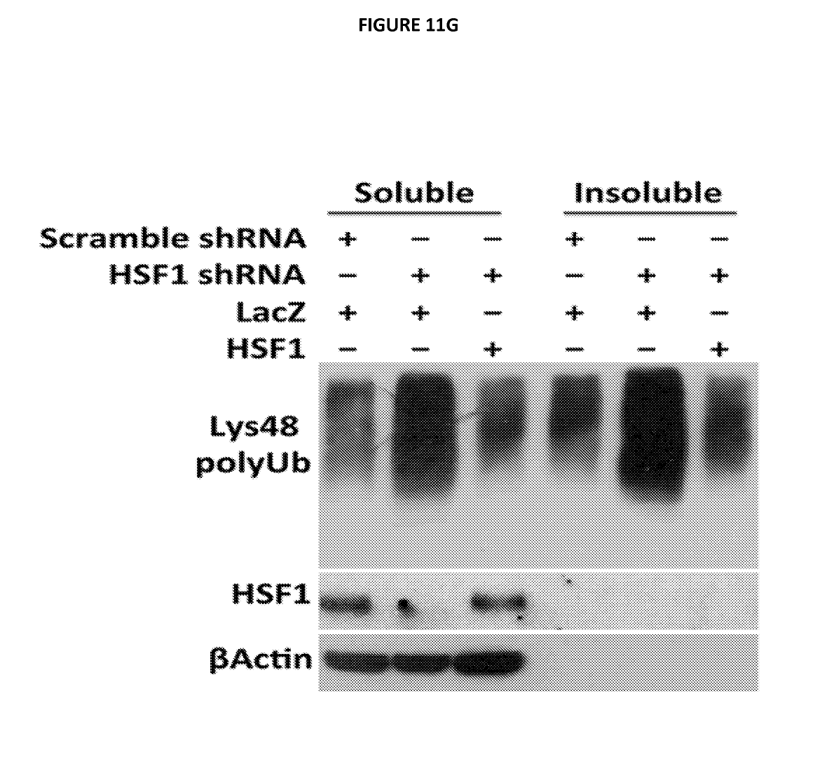

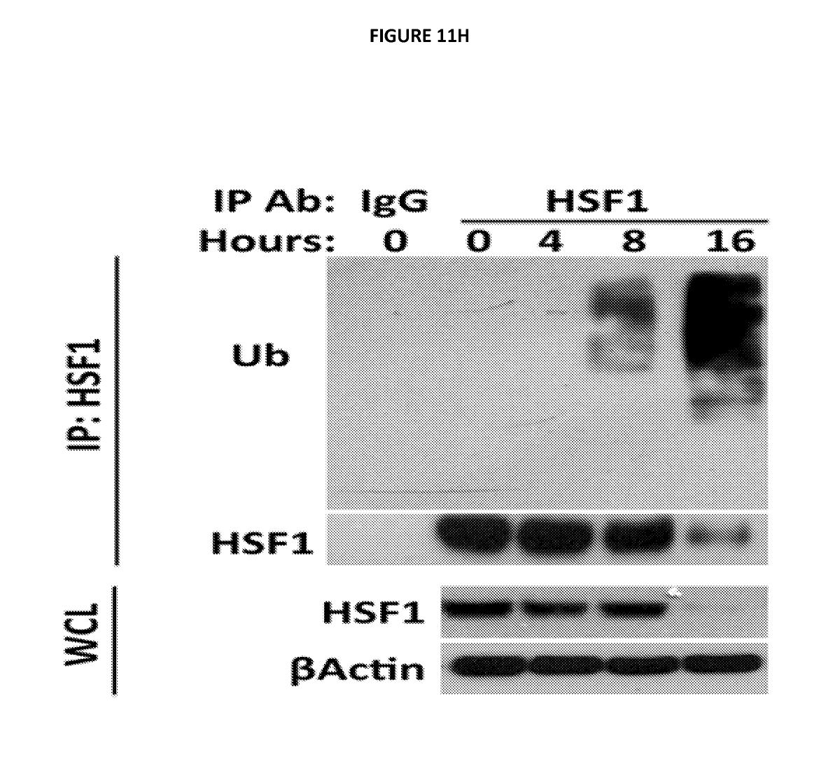

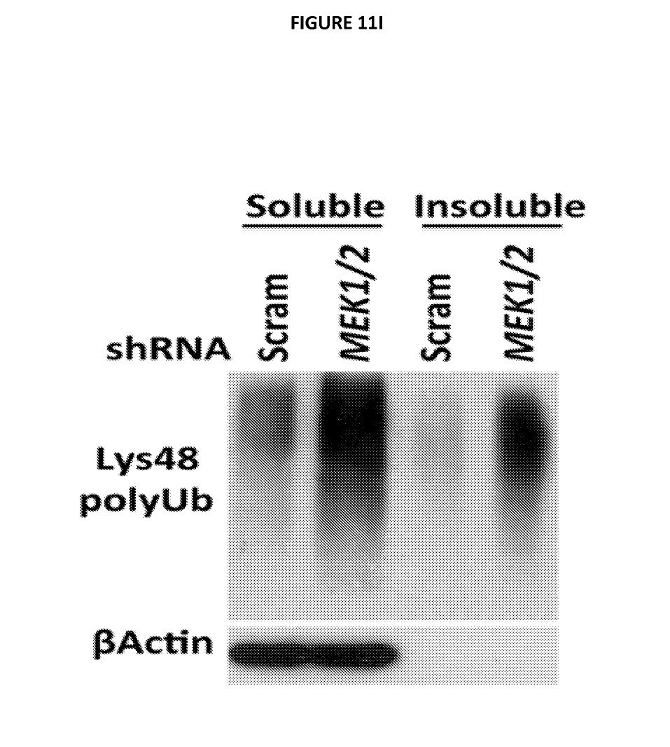

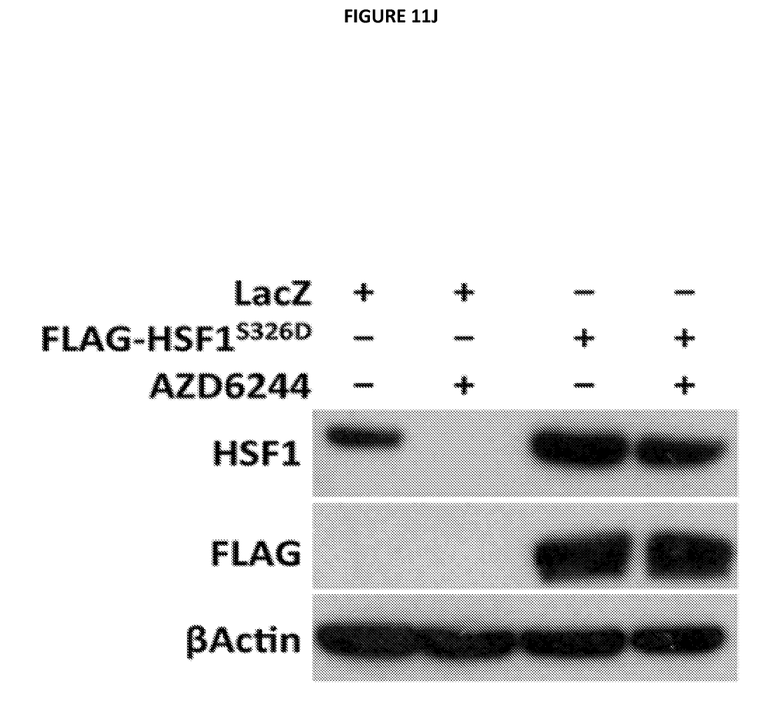

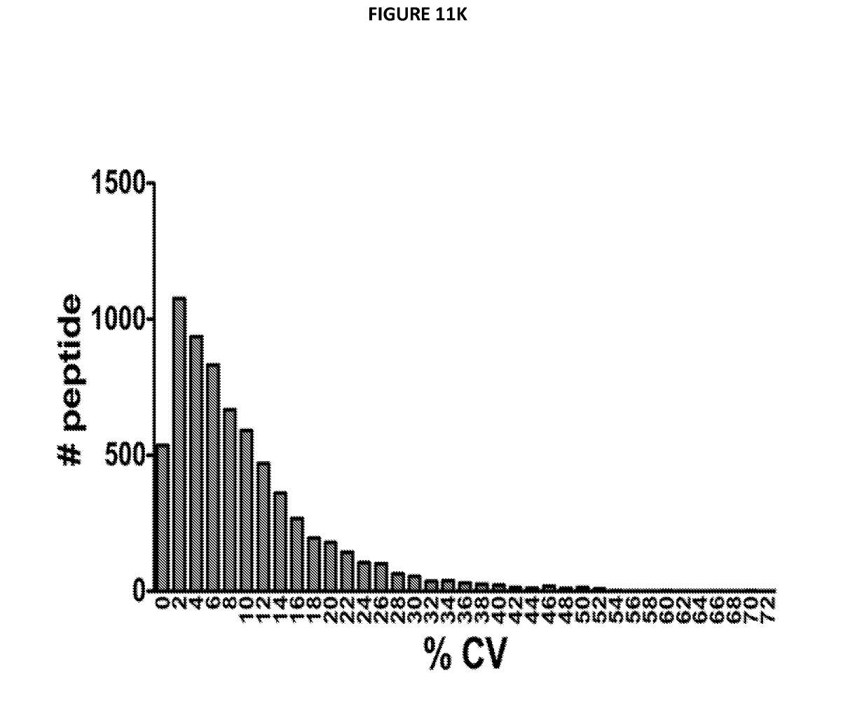

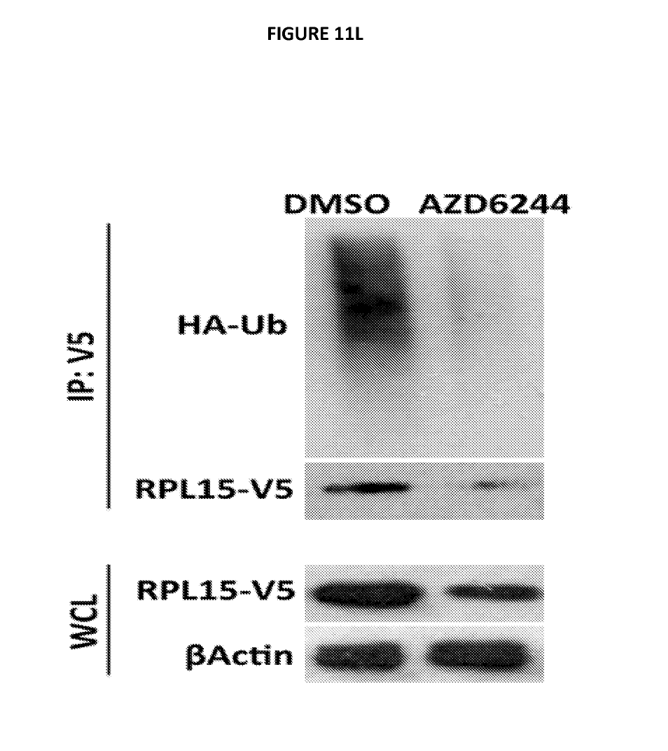

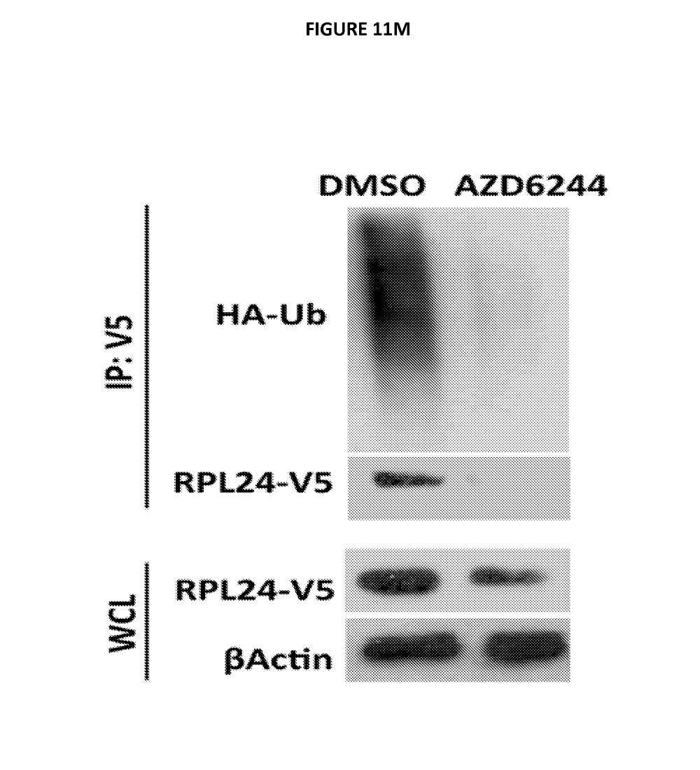

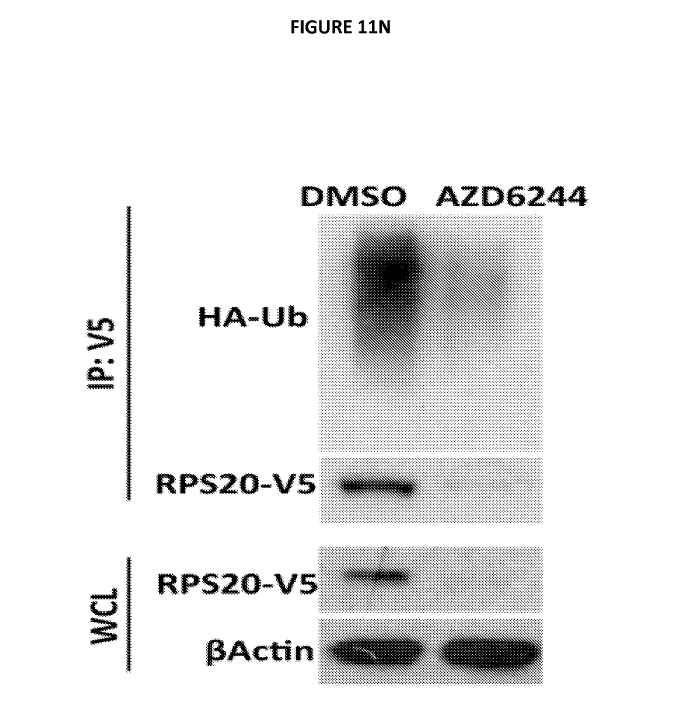

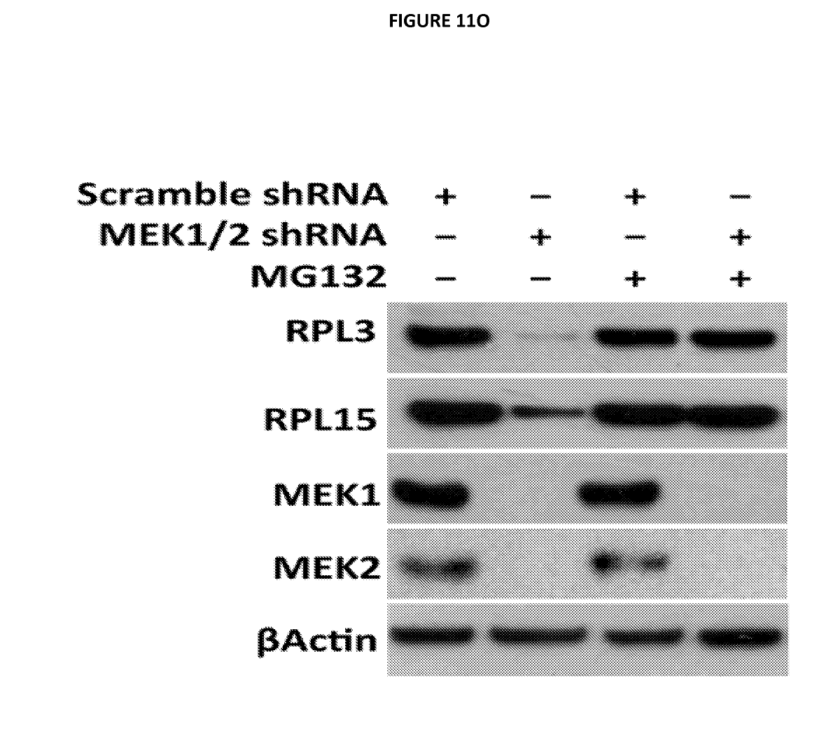

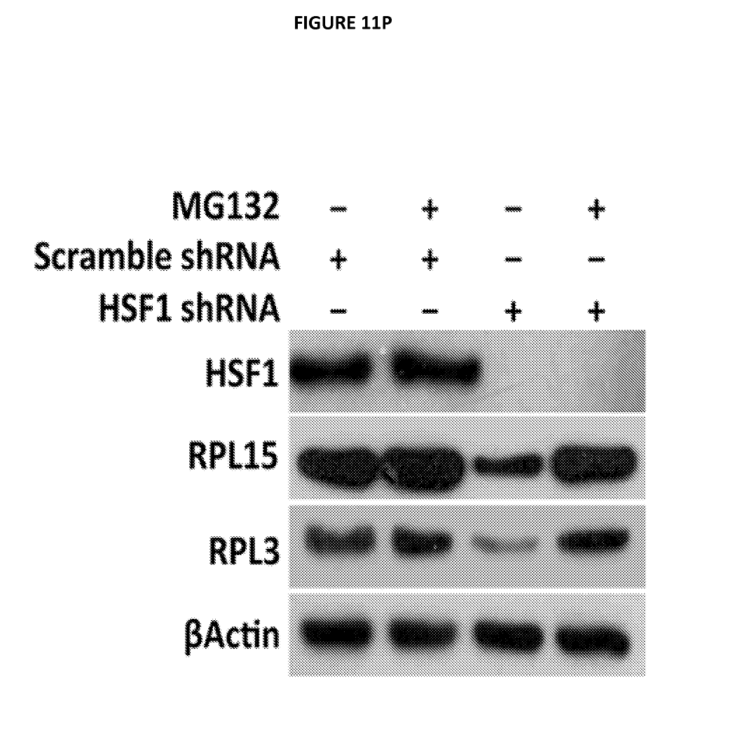

HSF1 could maintain cellular proteostasis via HSPs. To examine the impacts of HSF1 on protein folding, we employed the glucocorticoid receptor (GR) as a model. Proper GR folding depends on HSP90 and misfolded proteins are cleared by the ubiquitin-proteasome system (Taipale et al., 2010). HSF1 knockdown induced GR-GFP ubiquitination and depletion (FIG. 11A), indicating protein destabilization by HSF1 deficiency. This resulted from diminished cellular chaperoning capacity, as lysates of HSF1-deficient cells were less efficient in reactivating denatured luciferase (FIG. 11B) Similarly to HSF1 deficiency, MEK blockade depleted GR-GFP; and this depletion is not due to GFP instability or general expression changes, since co-expressed GFP was not affected (FIG. 4A). Instead, MEK blockade ubiquitinated GR-GFP (FIG. 4B). This is not due to impaired proteasomal function, as proteasome inhibition by Bortezomib caused GR-GFP accumulation and MEK inhibitors did not affect proteasomal activities (FIG. 11C-11E). In fact, AZD6244 and MEK knockdown both depleted chaperoning capacity (FIGS. 4C and 11F), revealing modulation of protein folding and stability by MEK.

In line with a key role of HSF1 in governing cellular proteome, HSF1 depletion induced protein Lys48-specific ubiquitination, a modification marking proteins for proteasomal degradation (Pickart and Eddins, 2004), in both detergent-soluble and -insoluble fractions (FIG. 11G). This change suggests global protein destabilization. Consistent with HSF1 inactivation, AZD6244 diminished Ser326 phosphorylation, reduced HSPs, and induced overall ubiquitination (FIG. 4D). Overnight AZD6244 treatment also destabilized HSF1 (FIGS. 4D and 11H). MEK knockdown induced global ubiquitination as well (FIG. 11I). Importantly, AZD6244 failed to deplete HSF1 and provoke ubiquitination in cells stably overexpressing HSF1^{S326D} (FIGS. 4E and 11J), indicating a causative role of HSF1 inactivation in protein instability due to MEK inhibition. In vivo MEK inhibition also depleted HSPs and HSF1, and provoked ubiquitination in primary tissues (FIG. 4F).

To investigate ubiquitomic changes due to MEK inhibition, we conducted mass spectrometry (MS)-based analyses of ubiquitinated peptides enriched by a novel ubiquitin branch motif antibody (FIG. 4G; Kim et al., 2011). We compared the ubiquitomes of A2058 cells treated with and without AZD6244 for 8 hours. In total, 3,425 non-redundant ubiquitinated peptides, assigned to 1,715 distinctive proteins, were profiled (FIGS. 4H and 11K). AZD6244 both increased and decreased peptide ubiquitination (FIG. 4H). When a 2.5-fold cutoff was defined as the significant change, a collection of 76 non-redundant peptides were distinguished. These peptides represent 68 unique proteins that perform diverse molecular functions and engage in a wide array of biological processes (FIGS. 4I and 4J). Intriguingly, the most enriched pathway was translation elongation (FIG. 4J) Analyses of these 68 proteins revealed a functional association network encompassing three subnetworks (FIG. 4K). Of particular interest is the “translation” subnetwork that consists of 7 ribosomal subunit proteins, highlighting a prominent impact of MEK on ribosome machinery. Moreover, embedded within the network are several oncogenes and tumor suppressors, including c-MYC, Cyclin D1, HIF1A, TP53, and NF1 (FIG. 4K). Although the mechanisms whereby MEK regulates these key players in oncogenesis are likely multifaceted, accumulating evidence has implicated HSPs in modulating their stabilities (Isaacs et al., 2002; Müller et al., 2004). Thus, MEK could impact these proteins at least in part via HSF1.

To validate our MS findings, we elected several target proteins. Torsin-1A interacting protein 2 (TOR1AIP2) and ribosomal protein L3 (RPL3) exhibited 61.0- and 13.7-fold increases, respectively, in ubiquitination. To facilitate detection, we expressed V5-tagged TOR1AIP2 and RPL3 proteins via a constitutive promoter. AZD6244 treatment for 8 hours did not alter levels of both V5-tagged proteins but increased their ubiquitination (FIGS. 4L and 4M). Our MS results also revealed decreased ubiquitination of proteins including c-MYC, RPL15, RPL24, and RPS20. We confirmed reductions in both ubiquitination and total levels of endogenous c-MYC proteins (FIG. 4N) Similar results were also observed for V5-tagged RPL15, RPL24, and RPS20 (FIG. 11L-11N), suggesting shortened protein half-life. Indeed, proteasome blockade by MG132 prevented V5-RPL15 depletion by AZD6244 and revealed its elevated ubiquitination (FIG. 4O). Consistent with a critical role of HSF1 inactivation, both V5-tagged RPL15 and RPL3 proteins were highly ubiquitinated following HSF1 knockdown, and AZD6244 subtly affected this ubiquitination (FIGS. 4O and 4P). Importantly, MG132 prevented depletions of endogenous RPL15 and RPL3 by AZD6244 and MEK knockdown (FIGS. 4Q and 11O), confirming destabilization of ribosomal proteins by MEK deficiency. While HSF1 knockdown diminished endogenous RPL15 and RPL3, HSF1^{S326D} expression elevated their basal levels and protected them from AZD6244-induced depletions (FIGS. 11P and 4R). These findings together indicate that MEK inhibition inactivates HSF1 to deplete cellular chaperoning capacity. In consequence, protein destabilization and ubiquitomic imbalance ensue.

### Example 7. MEK Inhibition Provokes Aggregation and Amyloidogenesis in Malignant Cells

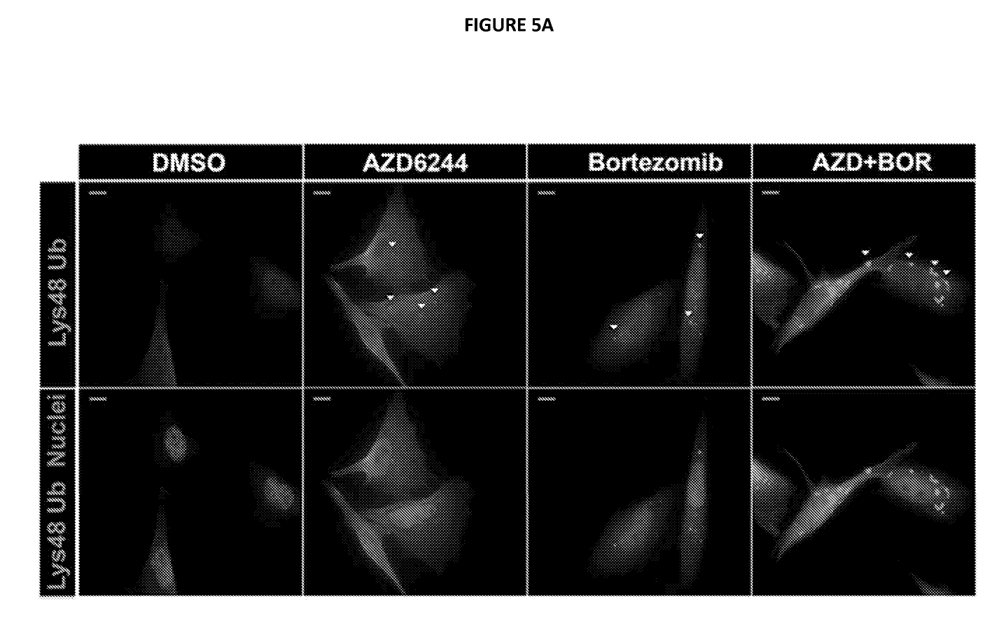

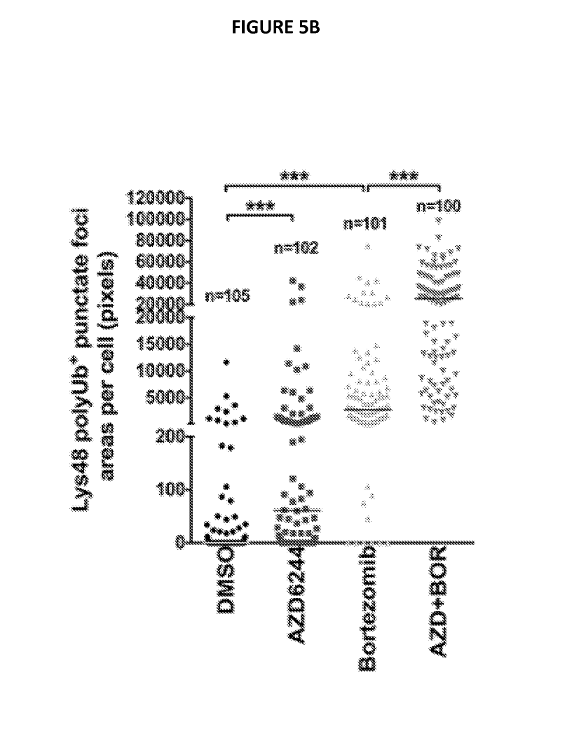

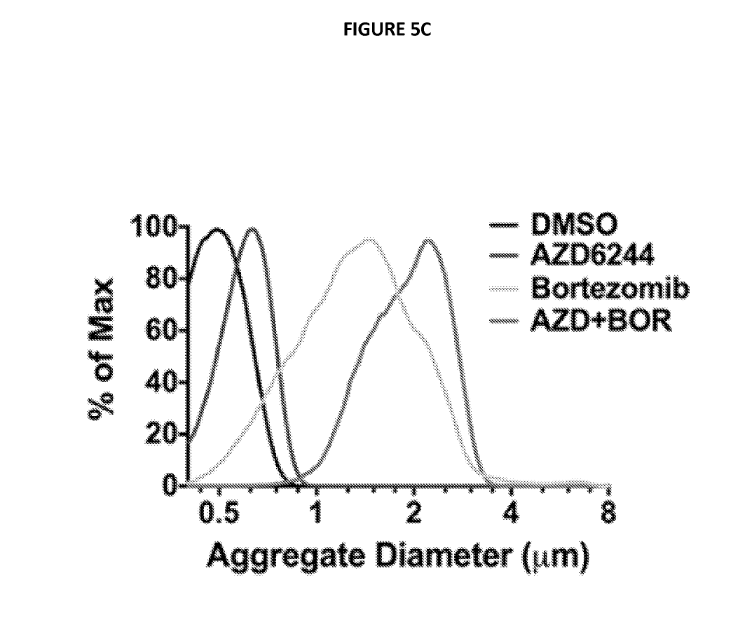

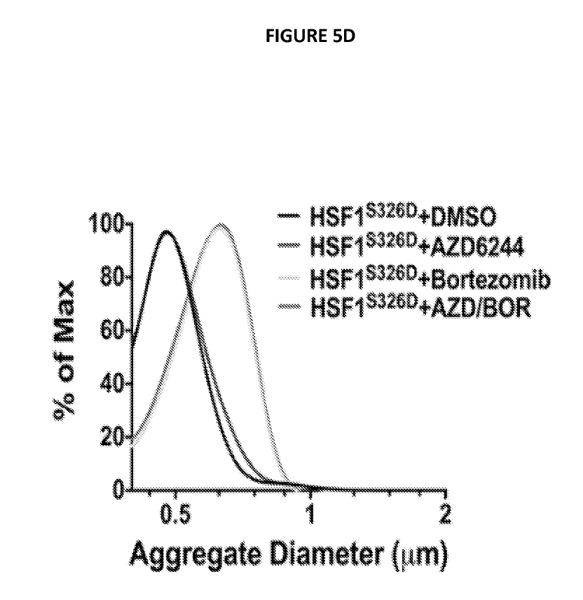

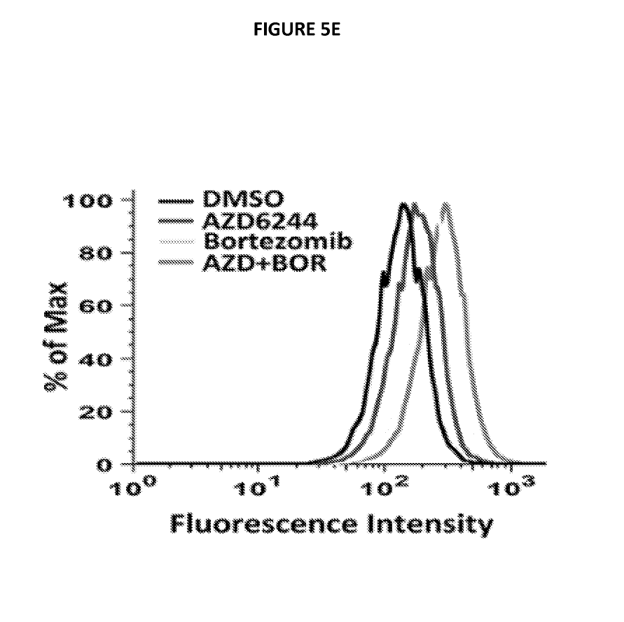

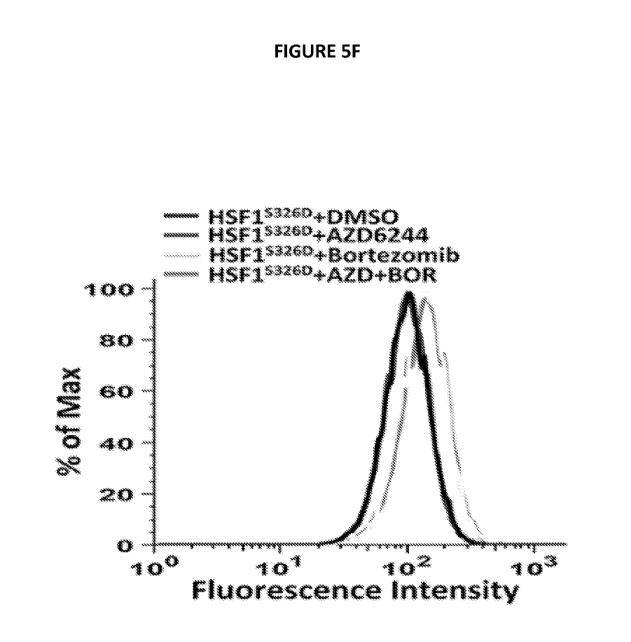

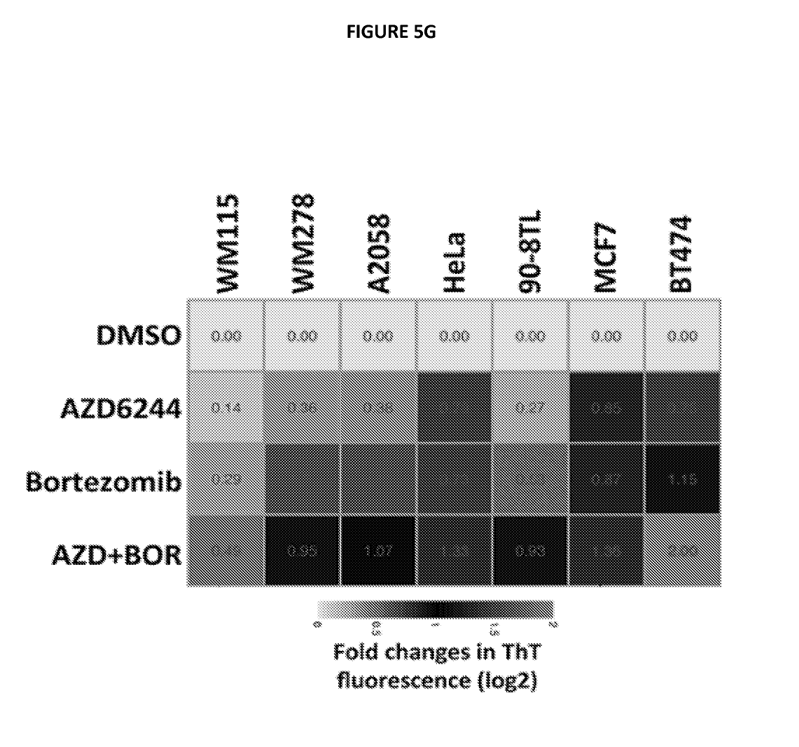

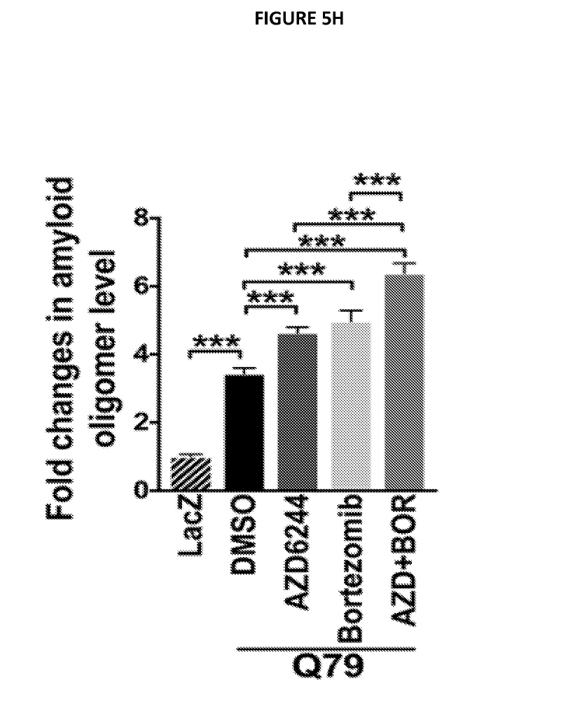

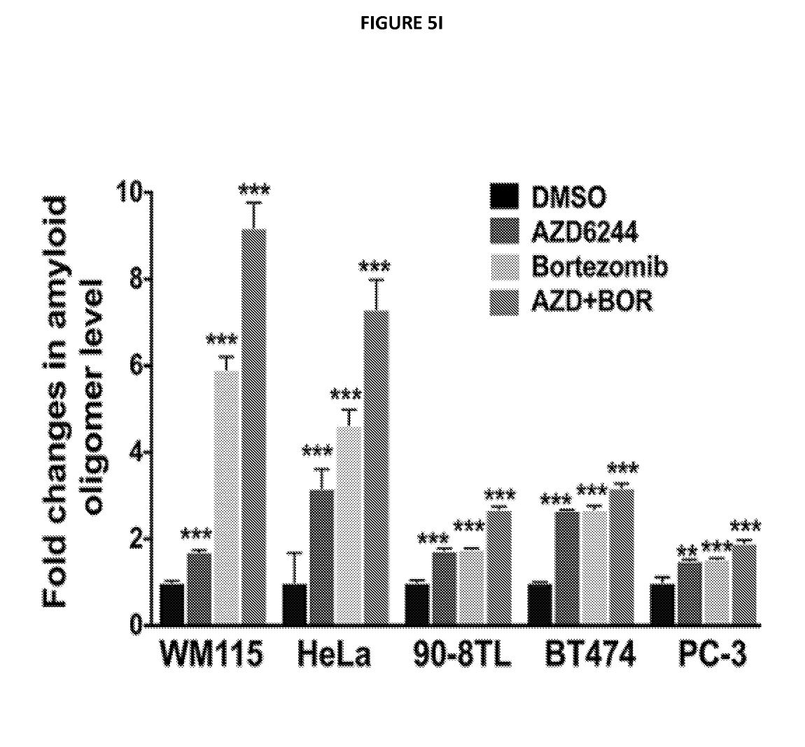

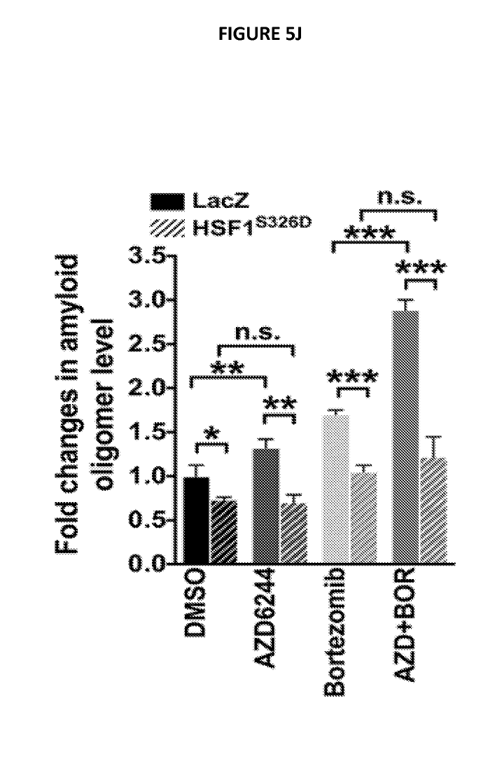

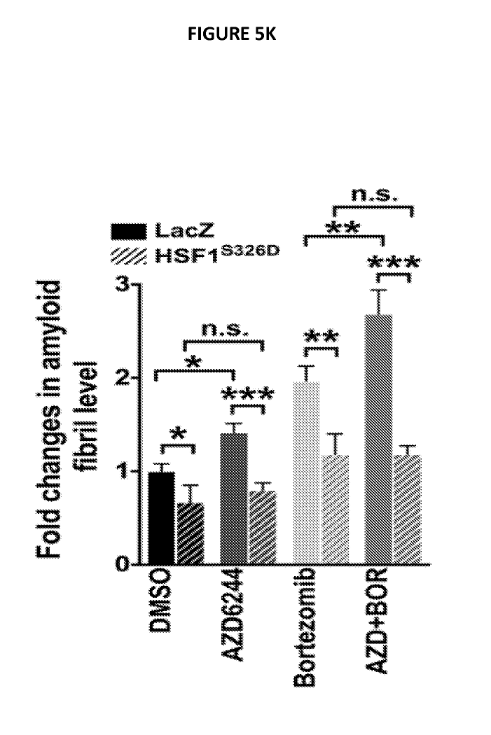

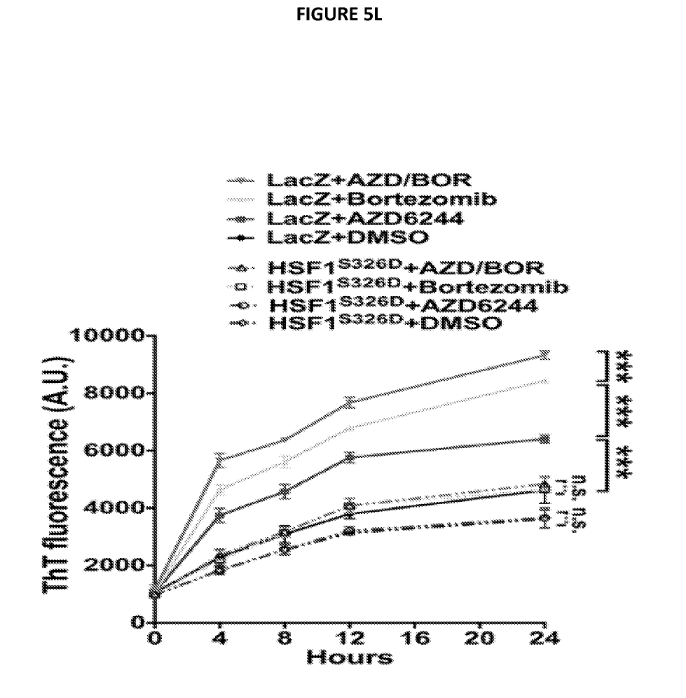

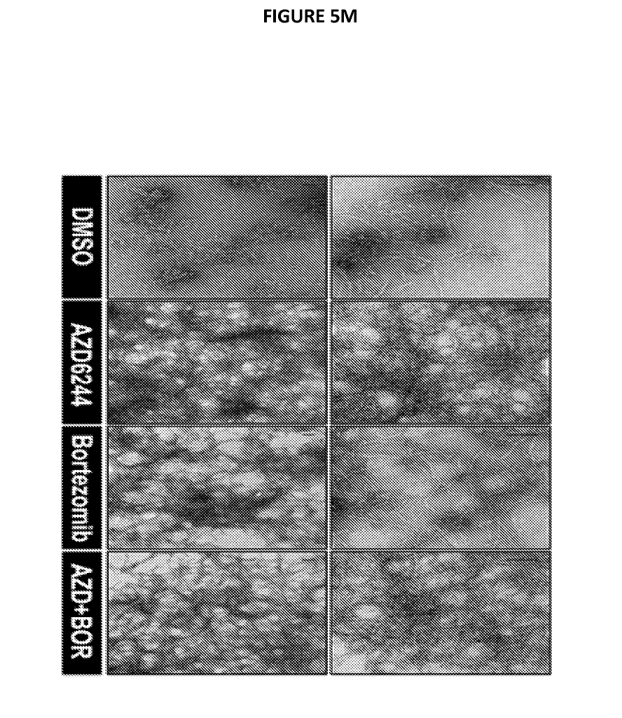

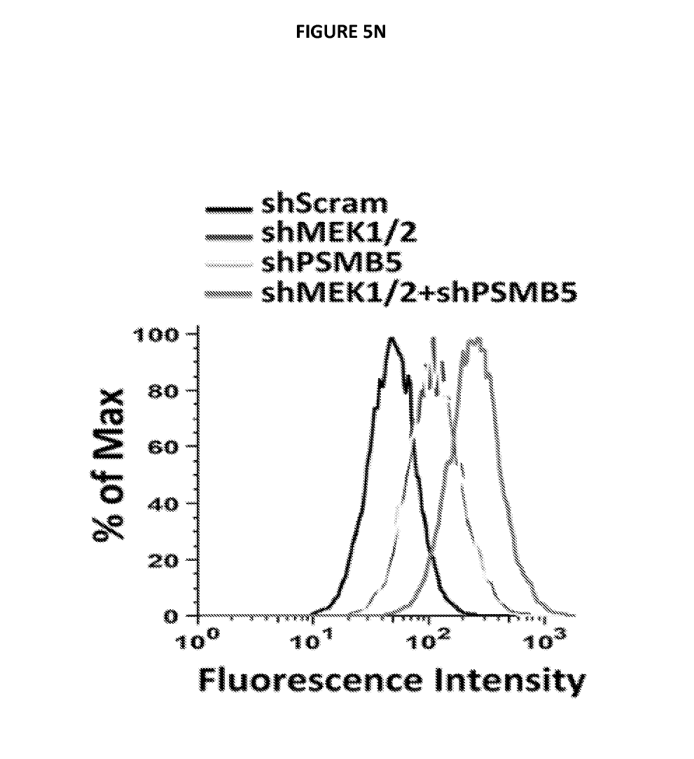

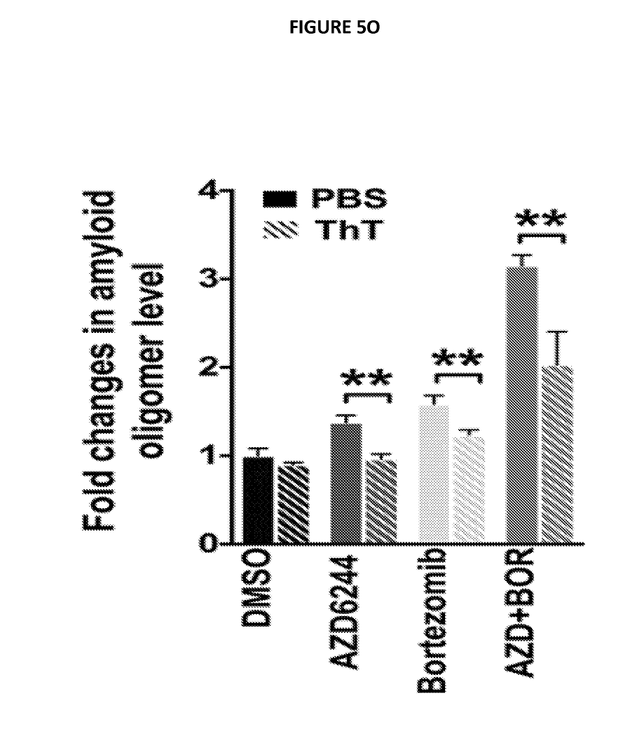

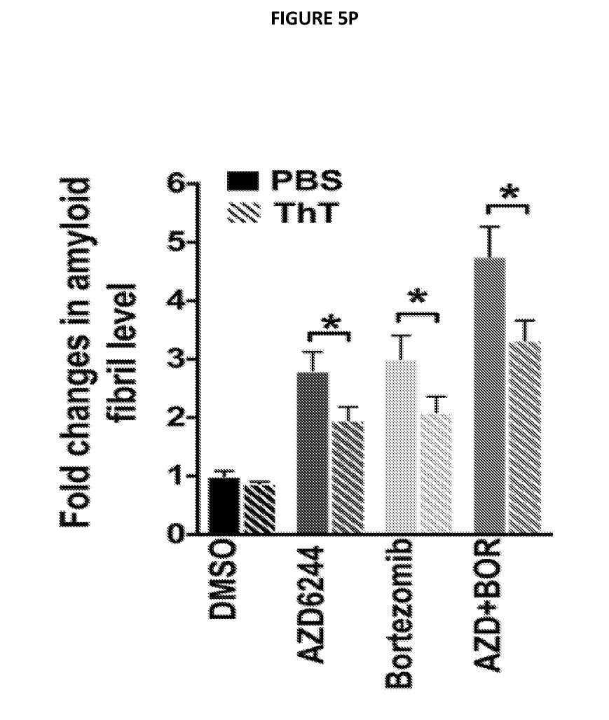

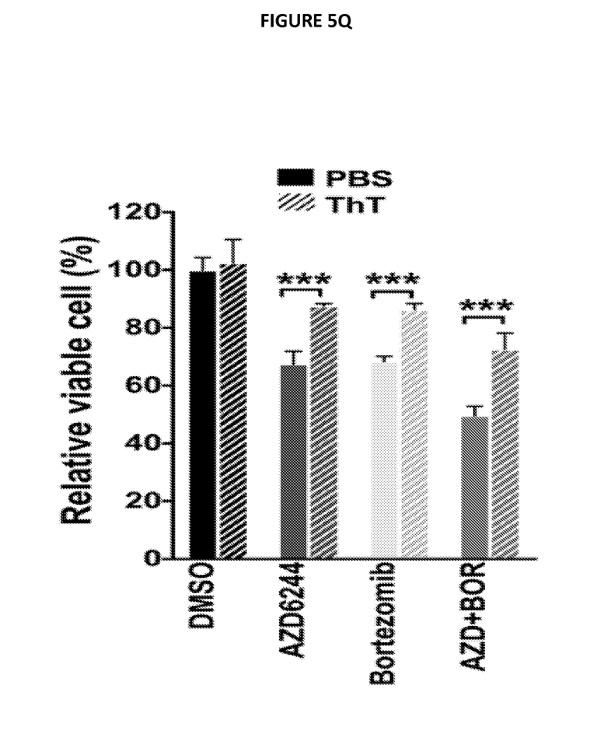

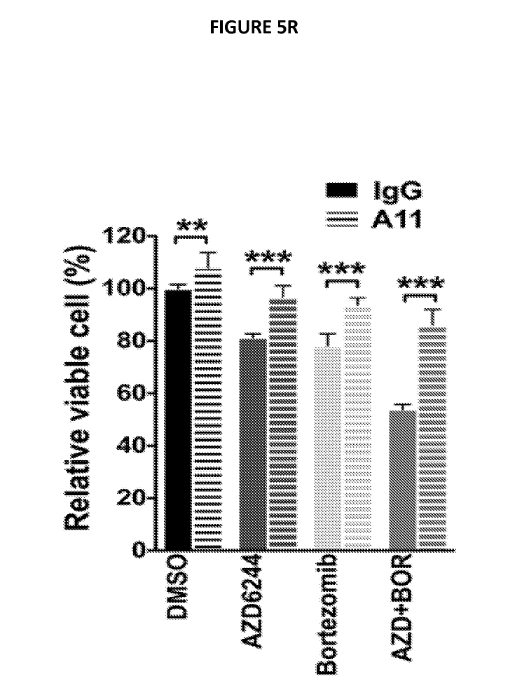

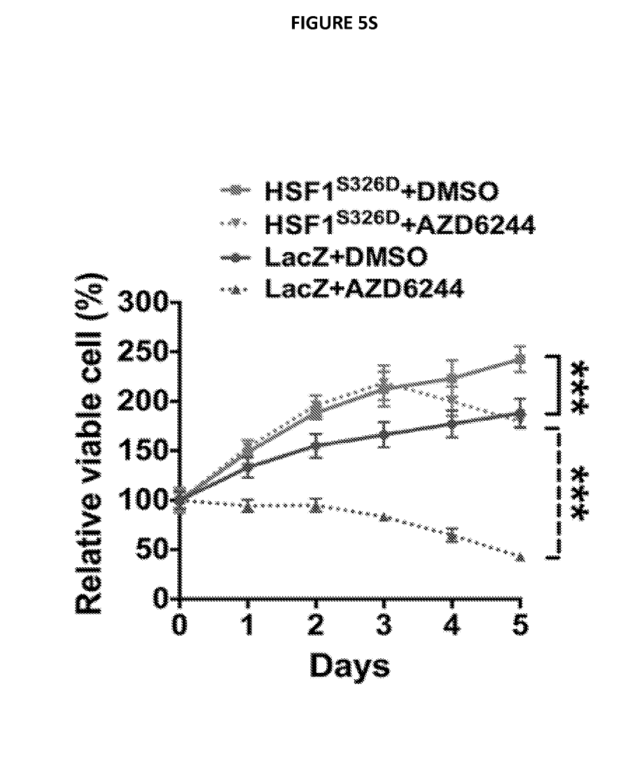

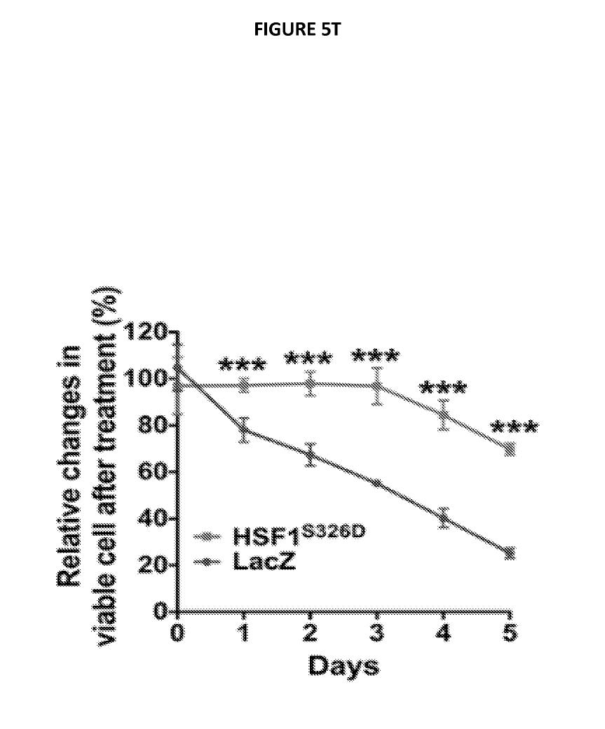

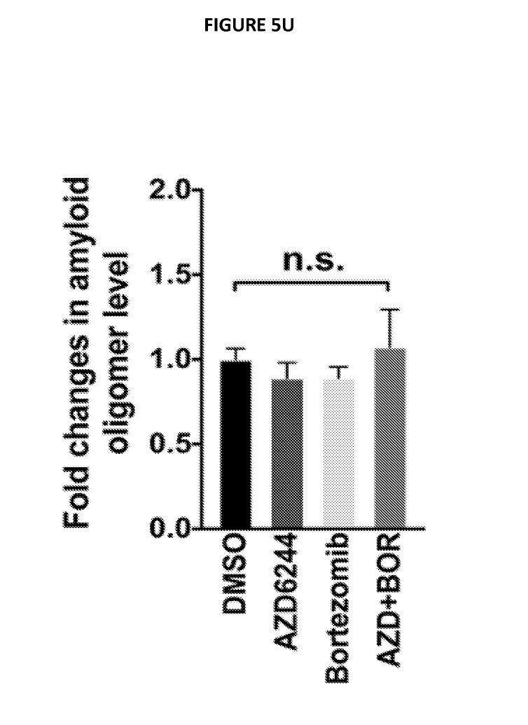

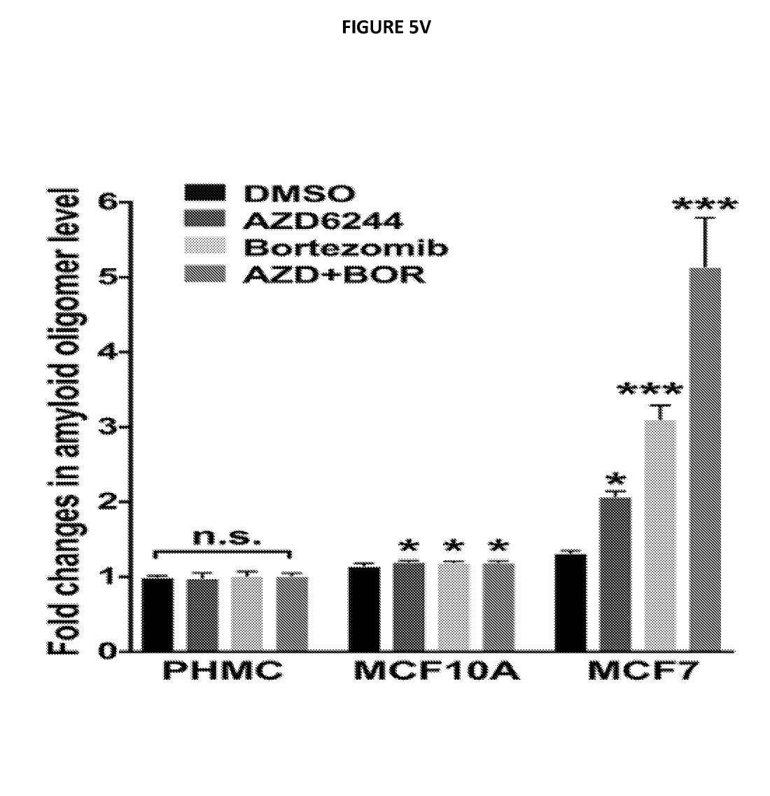

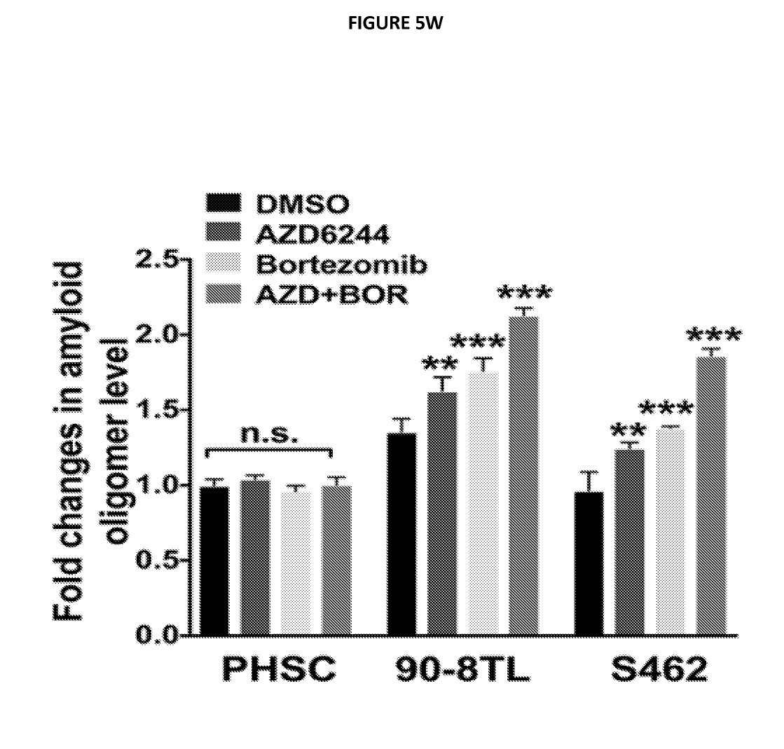

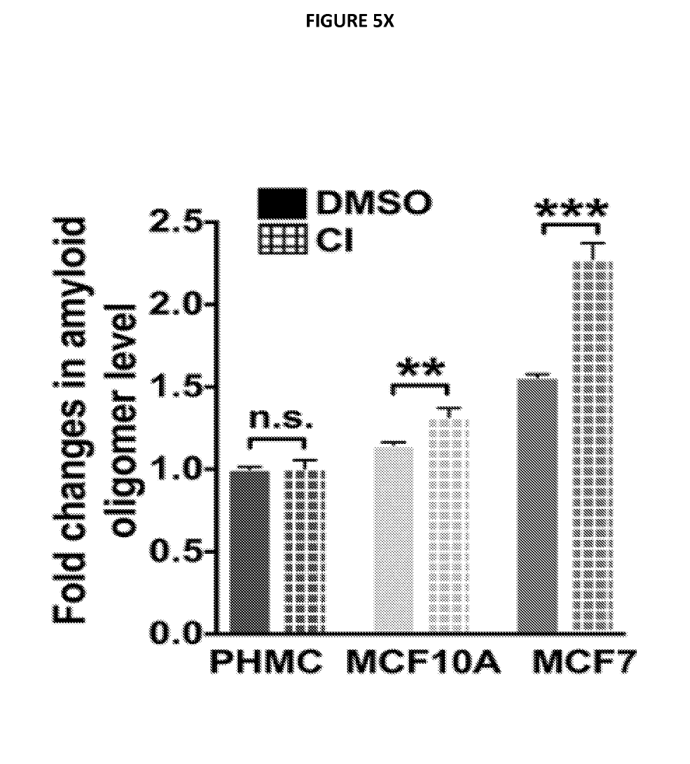

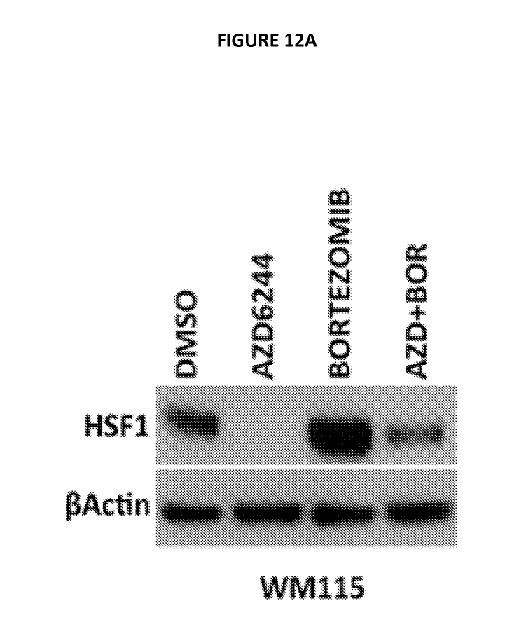

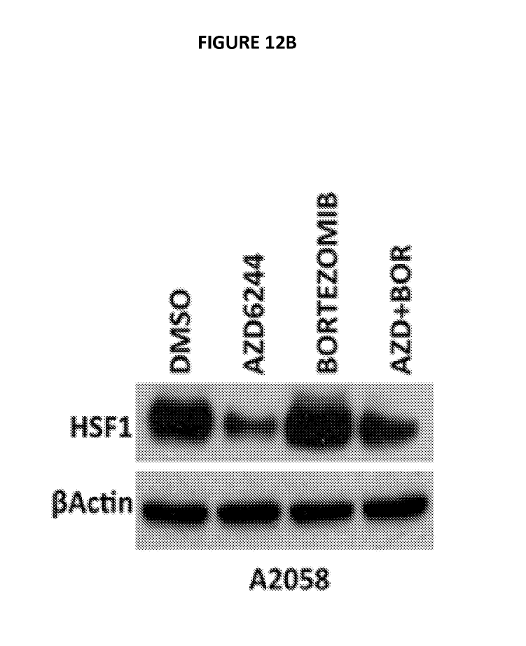

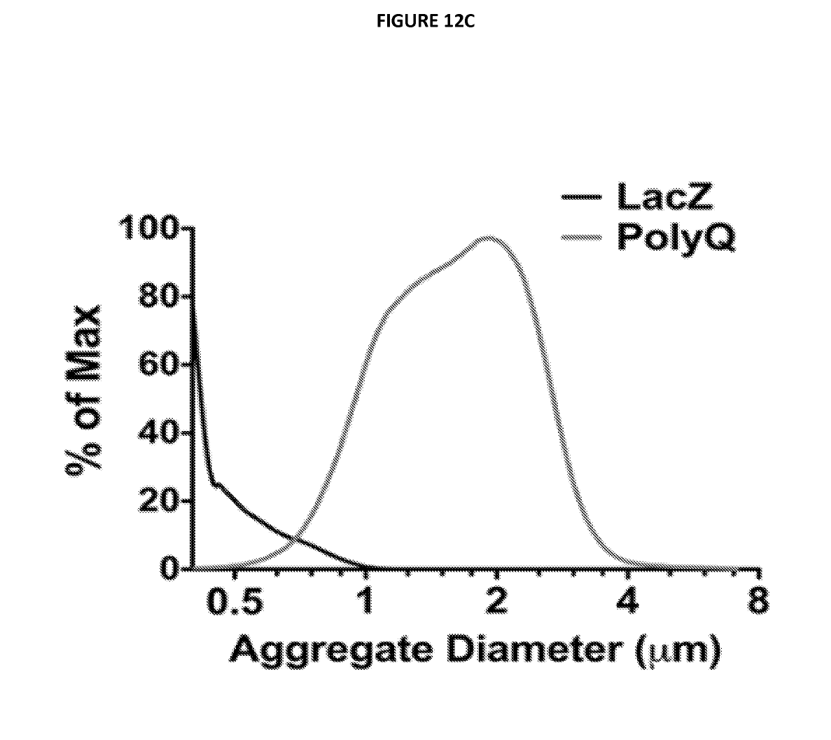

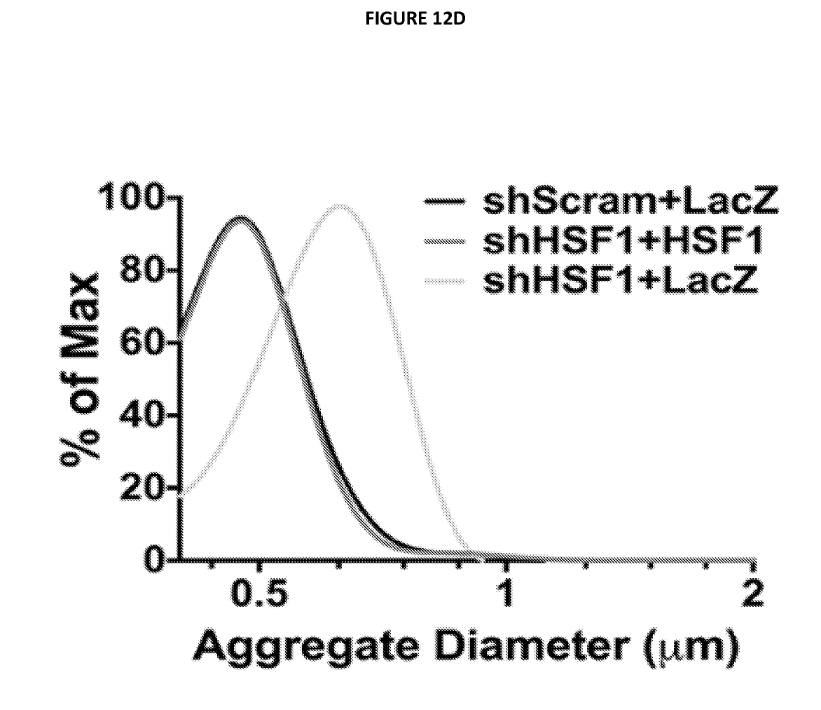

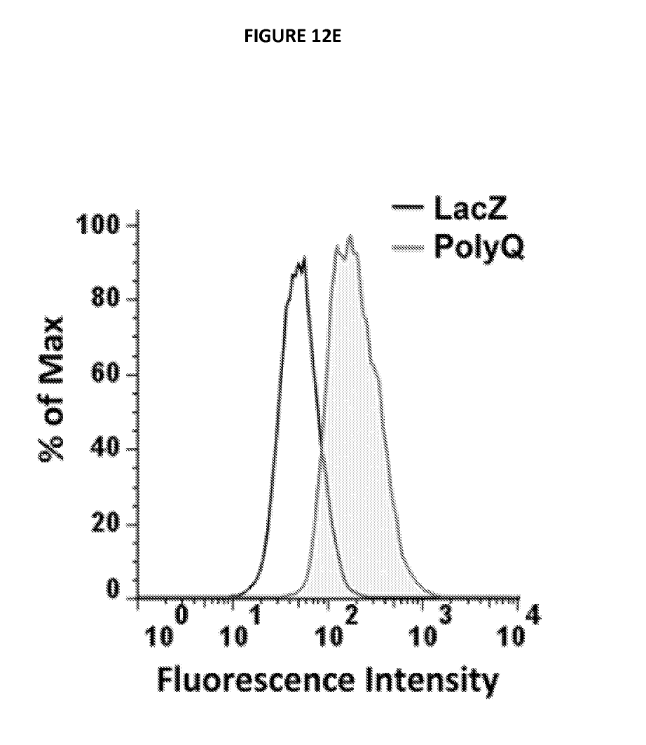

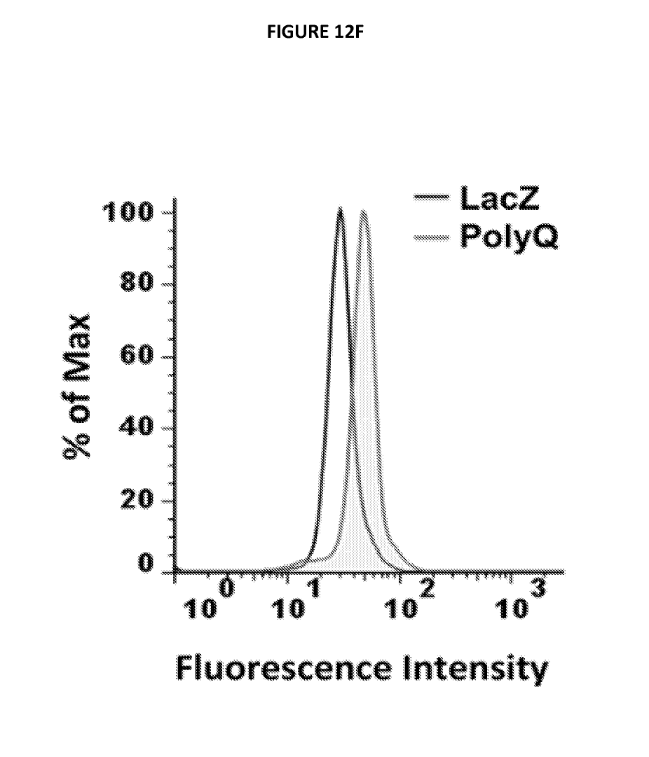

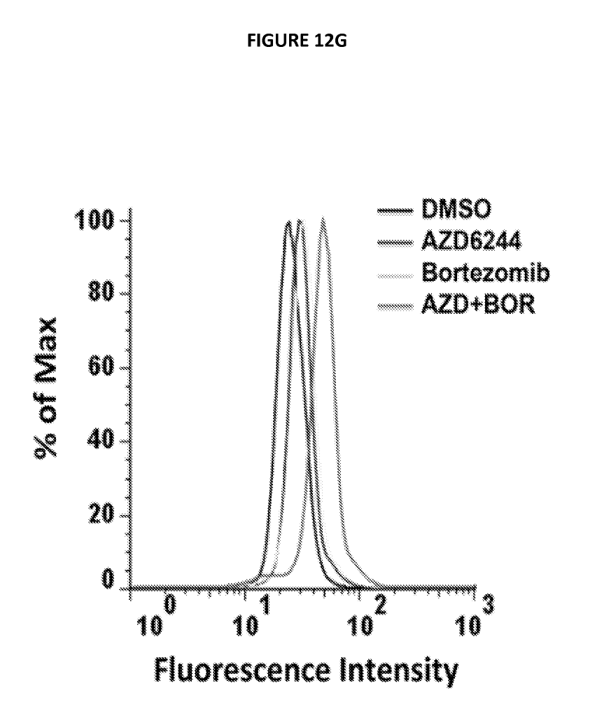

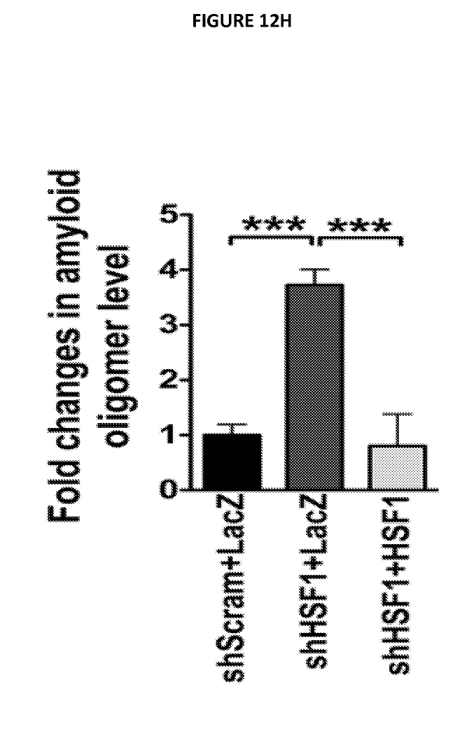

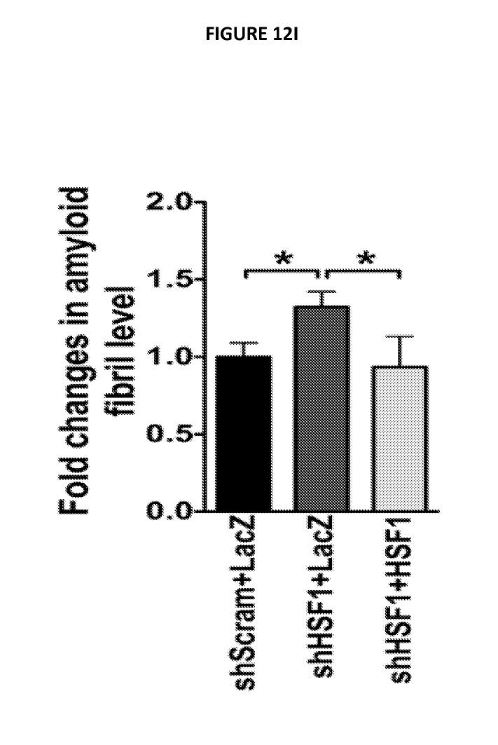

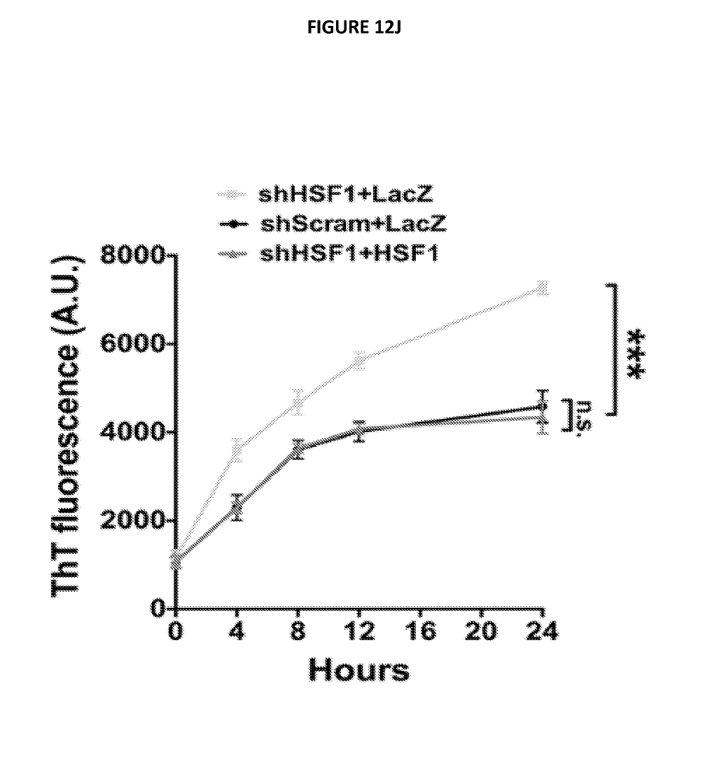

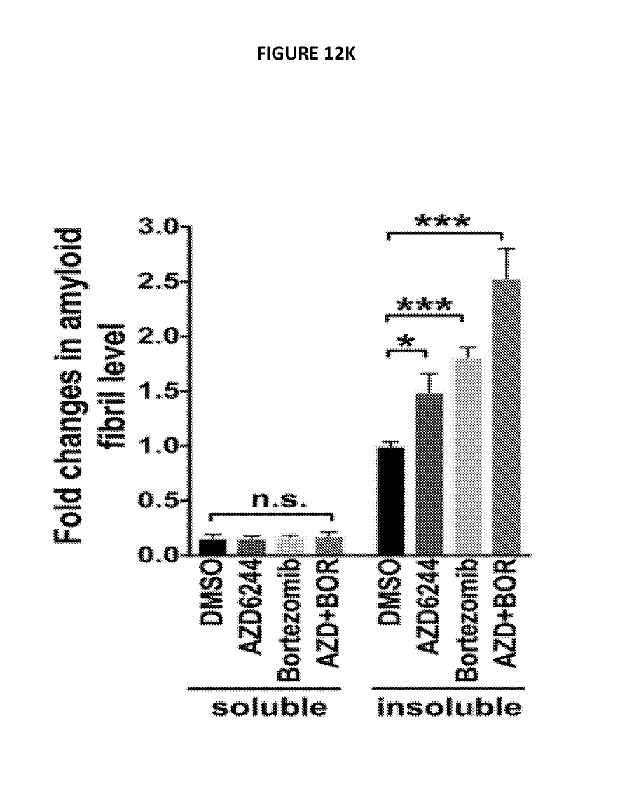

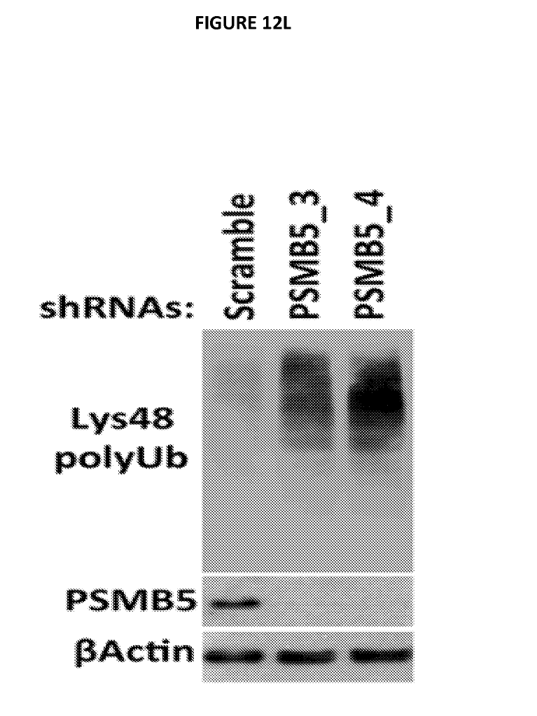

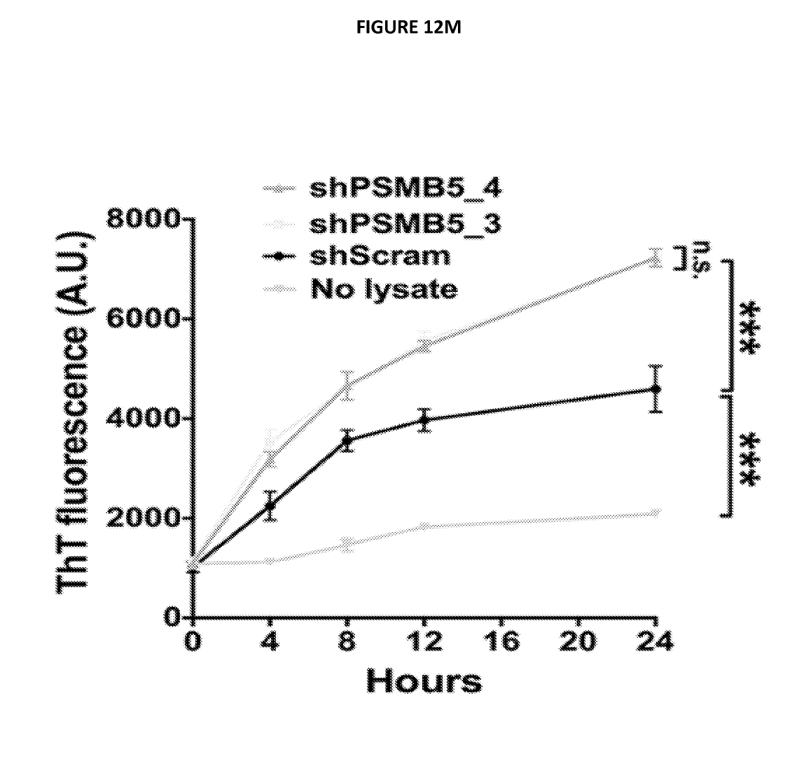

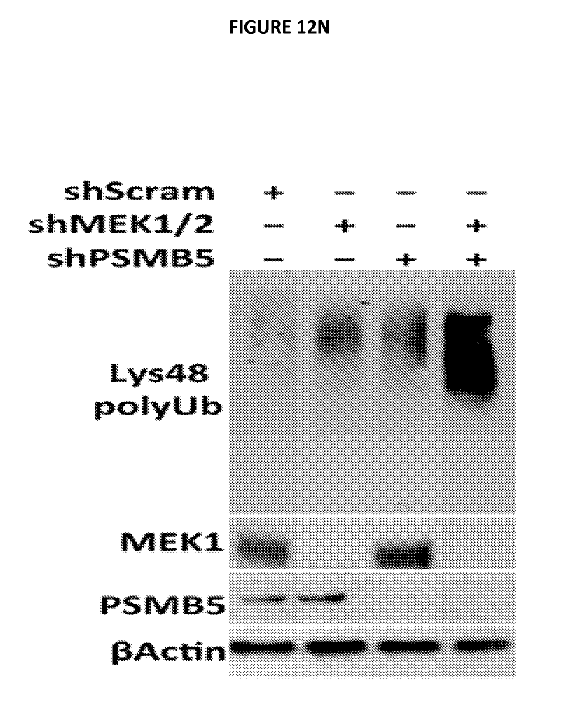

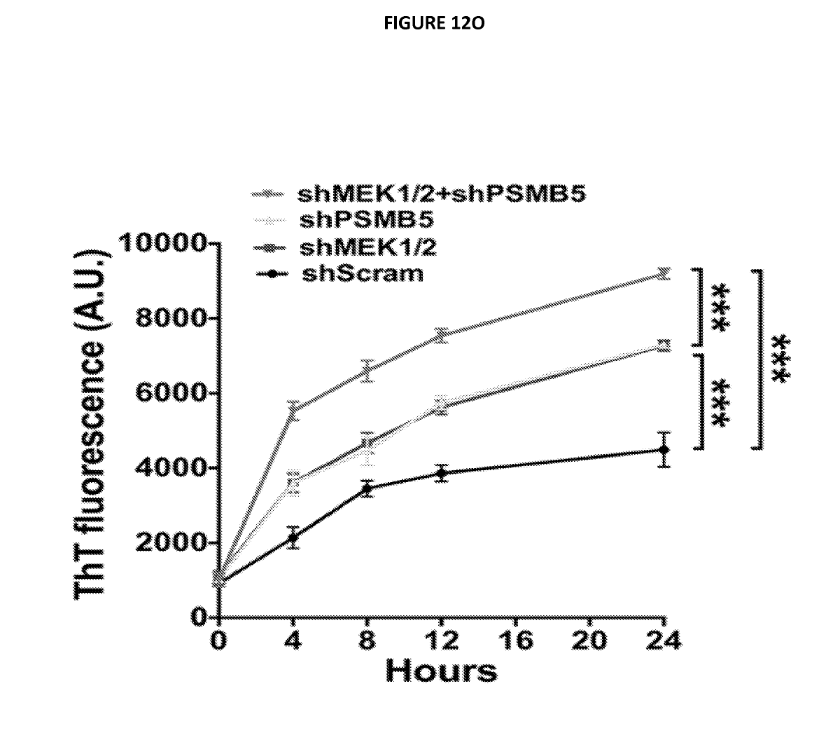

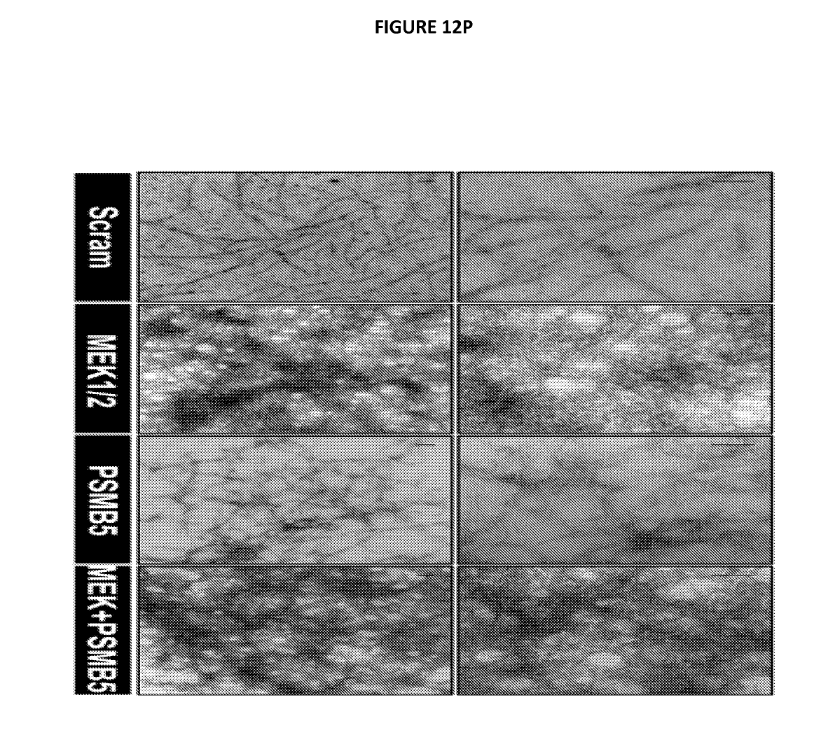

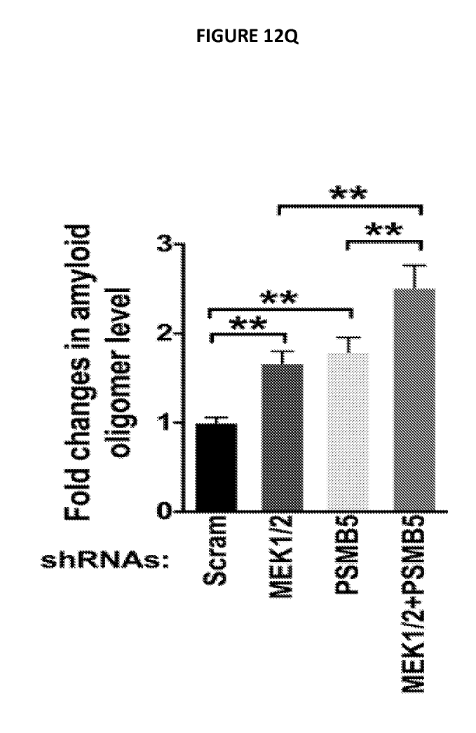

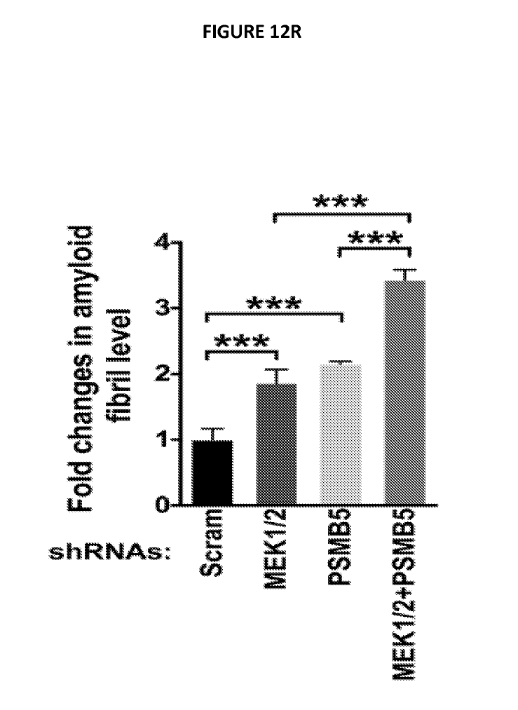

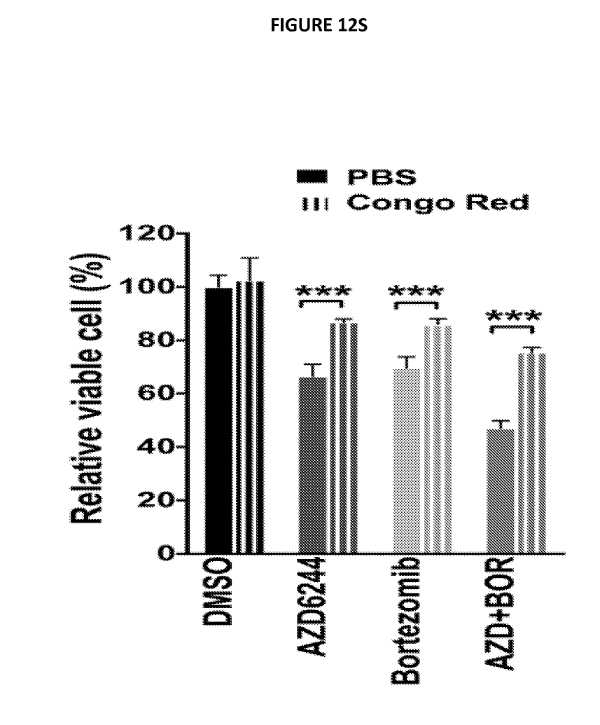

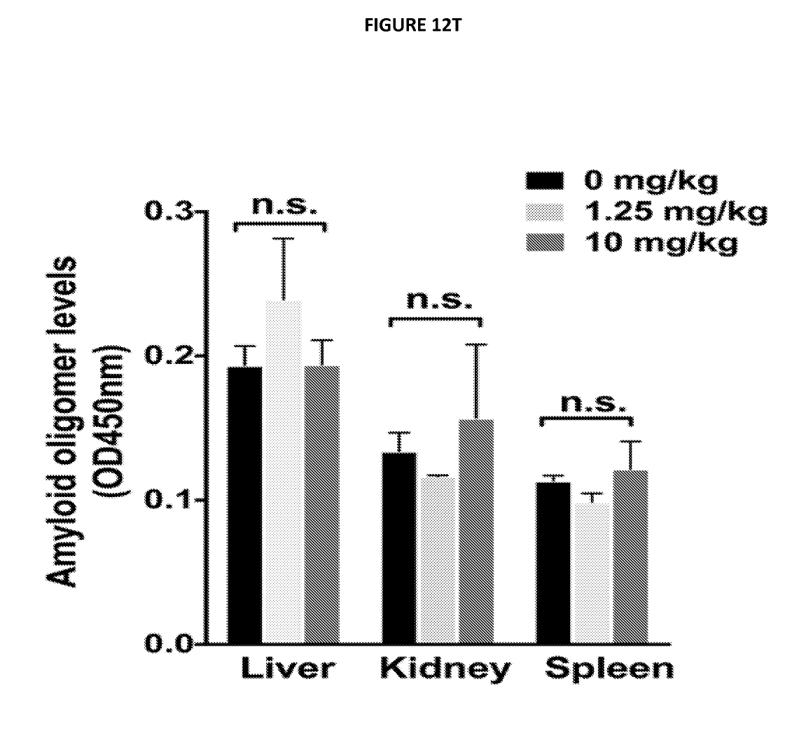

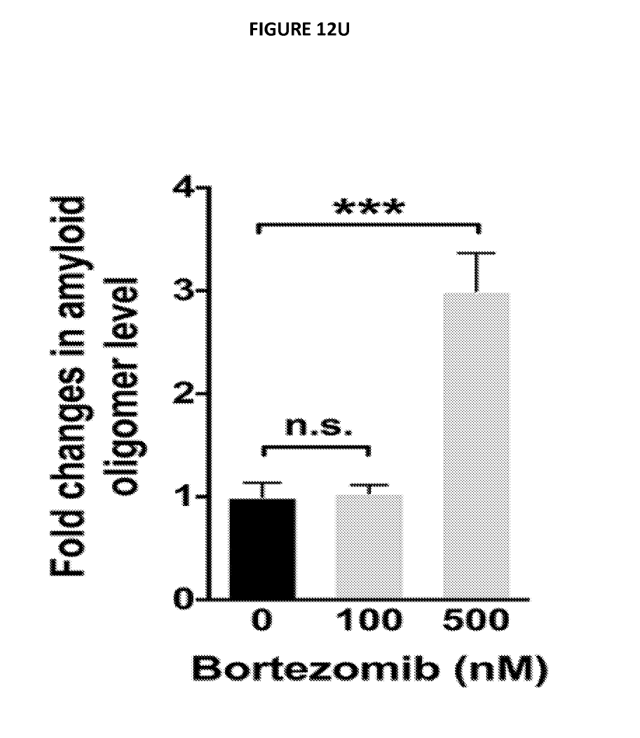

Increased ubiquitination in detergent-insoluble fractions suggests protein aggregation (FIG. 4D). To demonstrate this, we performed ubiquitin immunostaining Melanoma cells were treated with Bortezomib and AZD6244 for 24 hours to provoke evident aggregation. As expected, bright fluorescent punctate foci emerged in Bortezomib-treated cells (FIG. 5A-B), demarcating ubiquitin-containing aggregates. AZD6244 depleted HSF1 and, albeit to a lesser extent, induced punctate foci (FIGS. 12A-B and 5A-B). We theorized that blockade of proteasomal degradation of AZD6244-induced misfolded proteins would aggravate aggregation. Indeed, Bortezomib co-treatment augmented AZD6244-induced punctate foci (FIG. 5A-B). We further confirmed the impact of MEK on aggregation using an expanded polyglutamine tract protein (polyQ79) (Sanchez et al., 2003). Proteins with expanded polyQ fragments are aggregation-prone and causally related to neurodegenerative disorders (Orr and Zoghbi, 2007). PolyQ79-expressing cells, as expected, contained large aggregates (FIG. 12C). HSF1 knockdown and AZD6244 both enlarged polyQ aggregates; importantly, HSF1^{S326D} expression antagonized AZD6244-induced aggregate enlargement (FIGS. 12D and 5C-D). Bortezomib also enhanced aggregation, and combined treatment produced the largest aggregates (FIG. 5C-D). Thus, both MEK and the proteasome suppress protein aggregation.

Aggregation-prone proteins can form amyloid fibrils (AFs) enriched for β-sheet structures (Eisenberg and Jucker, 2012). To assess whether HSF1 and MEK impact amyloid formation, we stained polyQ79-expressing cells with Thioflavin T (ThT) and Congo red (CR), two fluorescent dyes widely used to diagnose amyloids (Chiti and Dobson, 2006). PolyQ79 expression enhanced ThT and CR staining (FIGS. 12E and 12F), as expected. AZD6244, Bortezomib, and combined treatment further intensified this staining; and, HSF1^{S326D} expression antagonized the effect of AZD6244 (FIG. 5E-F). Treatments also enhanced ThT and CR staining of human tumor cell lines (FIGS. 5G and 12G), suggesting emergence of endogenous amyloid-like structures. The presence of soluble amyloid oligomers (AOs) confirmed amyloidogenesis. AOs are believed to constitute a key toxic species in neurodegenerative disorders and can be detected by the conformation-dependent antibody A11 (Chiti and Dobson, 2006; Glabe, 2008; Kayed et al., 2003). Treatments not only exaggerated AO induction by polyQ79, but also provoked genesis of endogenous AOs in human tumor cell lines (FIGS. 5H and 5I) Similarly to AZD6244, HSF1 depletion induced endogenous AOs and AFs (FIGS. 12H and 12I). A previously characterized antibody, OC, was used to detect AFs (Kayed et al., 2007). Again, HSF1^{S326D} expression suppressed AZD6244-induced amyloidogenesis (FIGS. 5J and 5K).

A unique feature of amyloids is their ability to seed AFs (Chiti and Dobson, 2006). In amyloid seeding experiments, lysates of HSF1-depleted cells accelerated formation of Aβ AFs (FIG. 12J) Similarly, lysates of cells treated with AZD6244, Bortezomib, and combination all exhibited augmented seeding efficacy (FIG. 5L), which was confirmed using OC antibodies (FIG. 12K). HSF1^{S326D} expression abolished the effect of AZD6244 (FIG. 5L). Furthermore, transmission electron microscopy revealed that while seeding with DMSO-treated cell lysates resulted into disordered rod-like protofibrils, a dense lattice of fiber-like structures emerged following seeding with AZD6244-treated lysates; in comparison, seeding with Bortezomib-treated lysates produced mature fibrils, and a compacted network of fibrils were assembled after seeding with lysates from combined treatment (FIG. 5M).

The amyloidogenic effects of AZD6244 and Bortezomib were validated genetically. Depletion of the β5 subunit (PSMB5) of the 26S proteasome, a primary target of Bortezomib (Oerlemans et al., 2008), caused accumulation of ubiquitinated proteins (FIG. 12L) Mimicking pharmacological inhibitors, genetic depletions of MEK, PSMB5, or both all provoked amyloidogenesis (FIGS. 12M-12R and 5N).

To determine whether amyloids contribute to inhibitor-induced toxicities, we blocked amyloidogenesis with ThT, which impedes amyloid fibrillization via physical binding (Alavez et al., 2011). In melanoma cells, ThT suppressed amyloid induction by inhibitors, and improved cellular growth and survival by 50% (FIGS. 5O, 5P, and 5Q). CR treatment and neutralization of AOs with A11 antibodies exerted similar protection (FIGS. 12S and 5R). Congruent with mitigated amyloidogenesis, HSF1^{S326D} expression not only stimulated the growth of melanoma cells but also rendered them refractory to MEK inhibition (FIG. 5S-T).

Surprisingly, AZD6244 did not induce AOs in primary mouse embryonic fibroblasts (MEFs) and tissues (FIGS. 5P and 12T). This is not due to inability to detect murine amyloids, as severe stress did induce AOs in murine cells (FIG. 12U). These results suggest that non-transformed cells may be more refractory to amyloidogenesis than malignant cells. To assess this, we compared AO levels in primary human mammary epithelial cells (PHMC), immortalized human mammary epithelial (MCF10A) cells, and tumorigenic mammary epithelial (MCF7) cells treated with AZD6244, Bortezomib, and the combination. Each of these three treatments caused marked AO induction in MCF7 cells, slight induction in MCF10A cells, and no induction in PHMC (FIG. 5V). A similar pattern was observed in primary human Schwann cells (PHSC) and their malignant counterparts, 90-8TL and S462 cells (FIG. 5W). Immortalized and transformed cells, except S462, also showed elevated basal levels of AOs (FIGS. 5V and 5W). Intriguingly, AO levels positively correlated with malignant states (FIG. 5X), supporting proteomic imbalance as an intrinsic feature of malignancy. We theorized that the lack of elevated basal AOs in S462 cells might be due to amyloid-associated toxicity. Indeed, blockade of cell death by a pan-caspase inhibitor elevated AO levels in immortalized and transformed cells, revealing heightened amyloidogenesis; in contrast, it did not elevate AOs in primary cells (FIGS. 5X and 5Y), supporting an absence of amyloidogenesis. These results indicate that malignant cells are distinctively susceptible to amyloidogenesis.

### Example 8. Combined Proteasome and MEK Inhibition Disrupts Tumor Proteostasis and Suppresses Malignancy

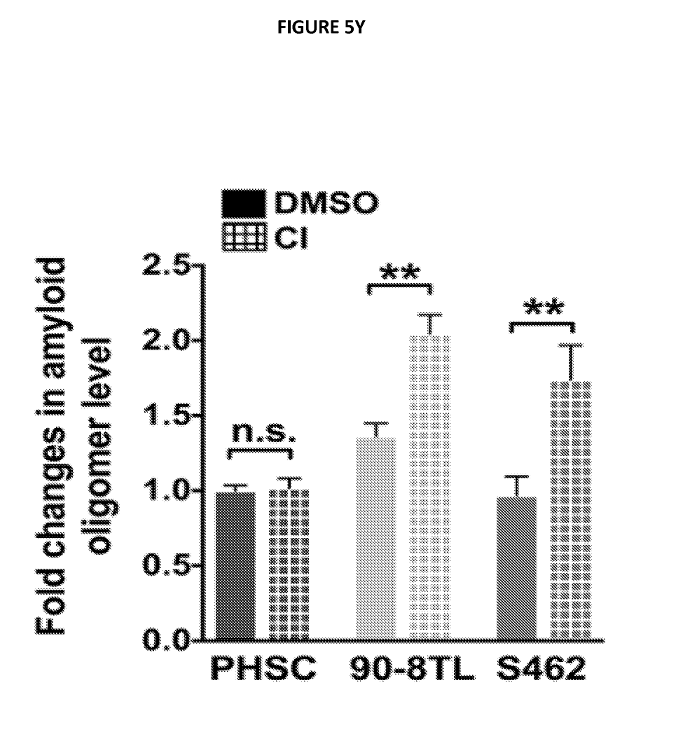

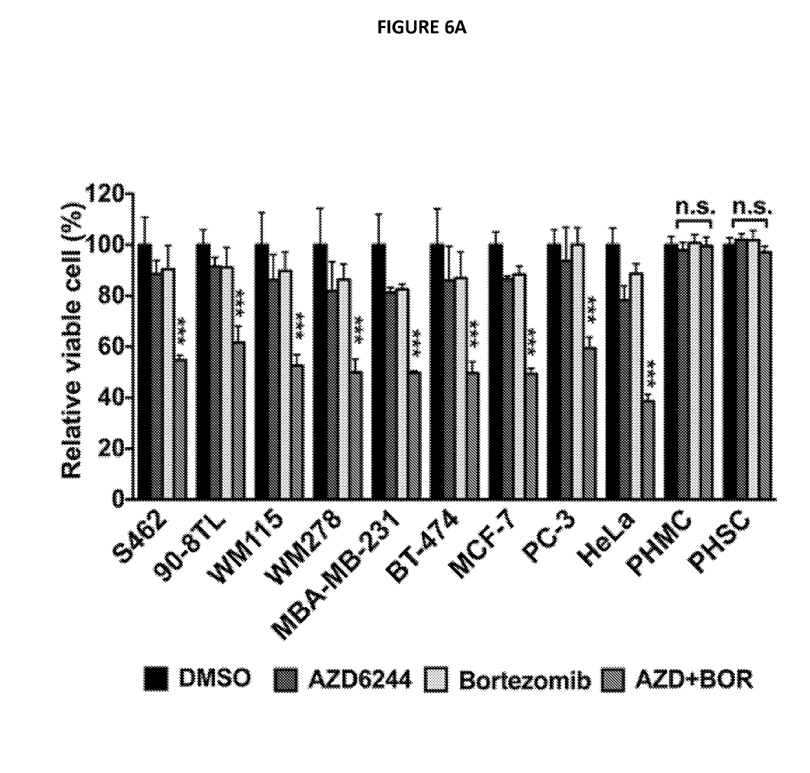

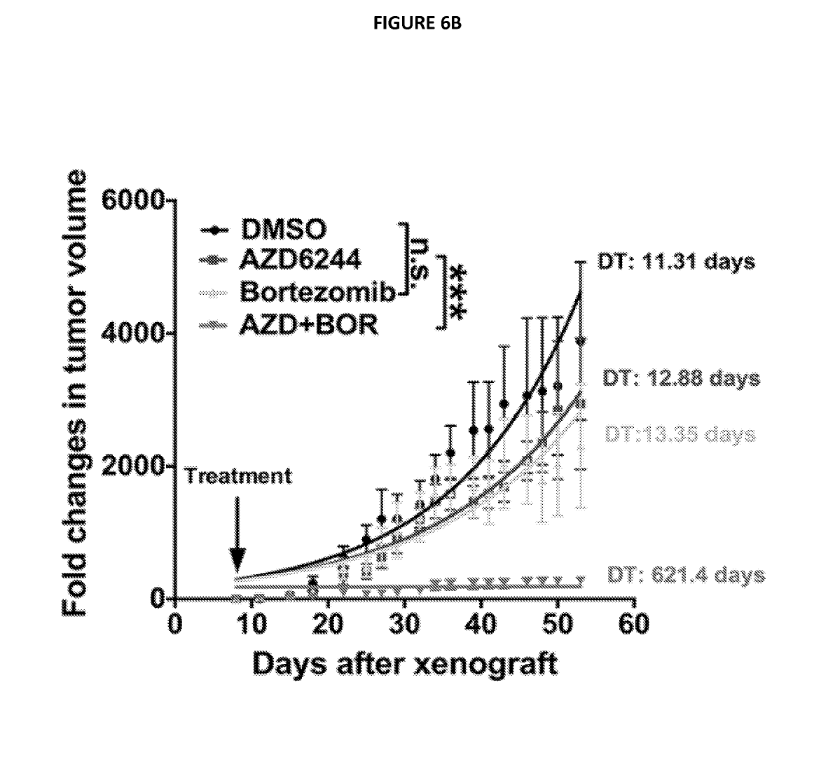

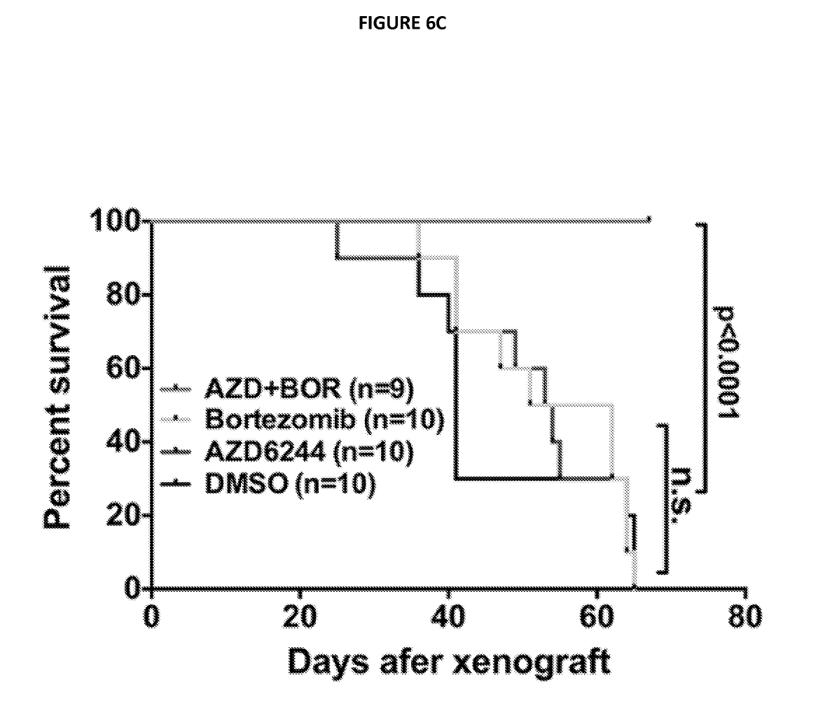

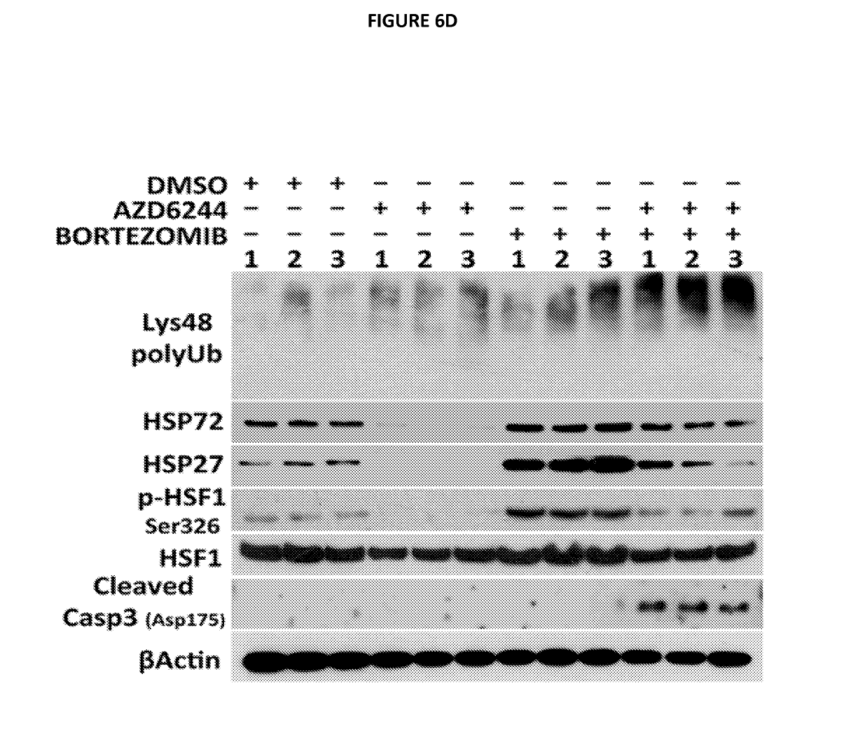

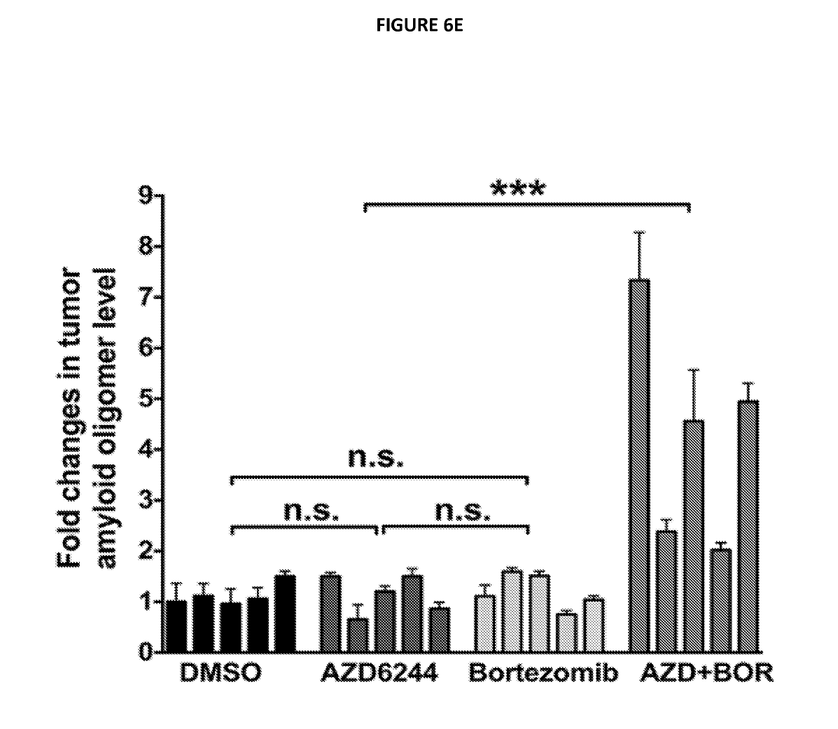

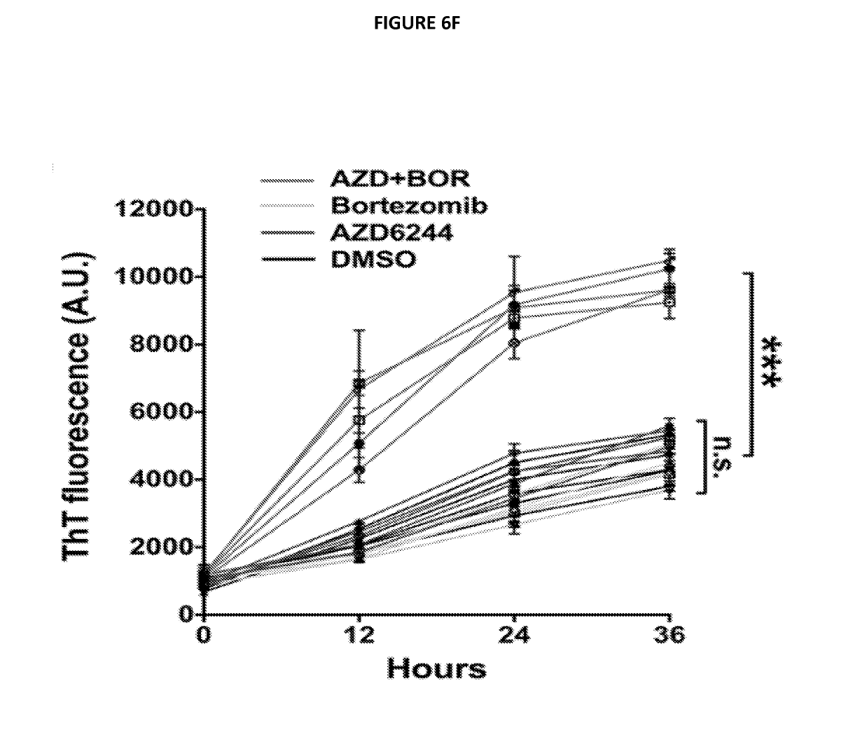

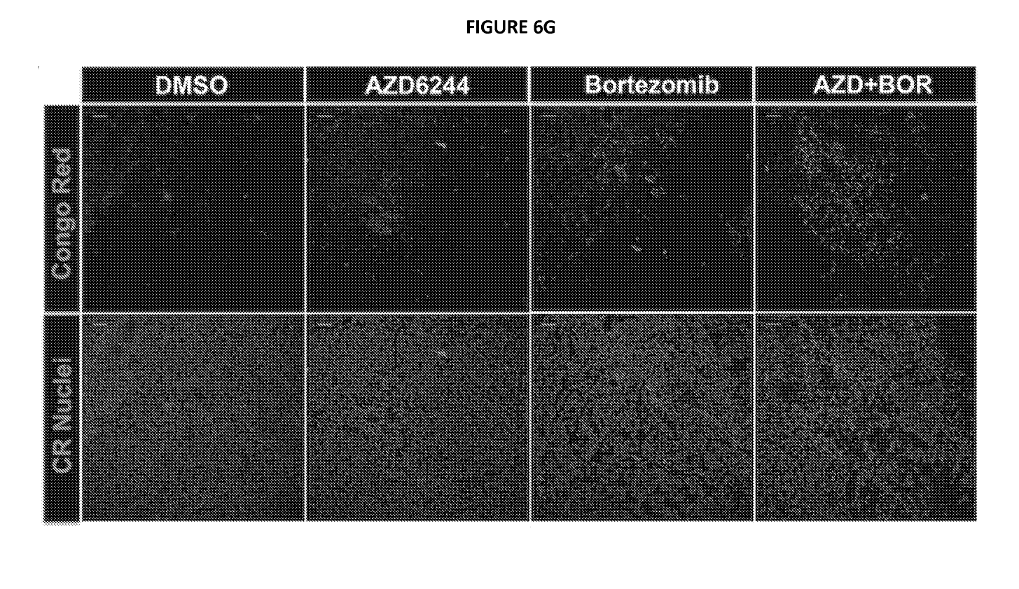

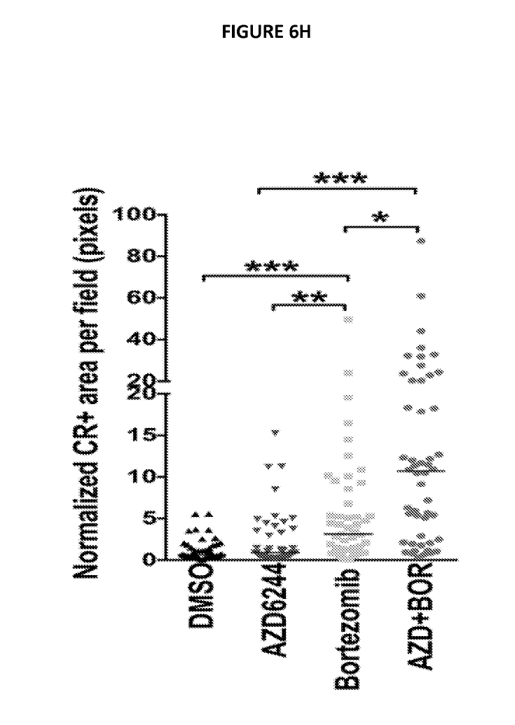

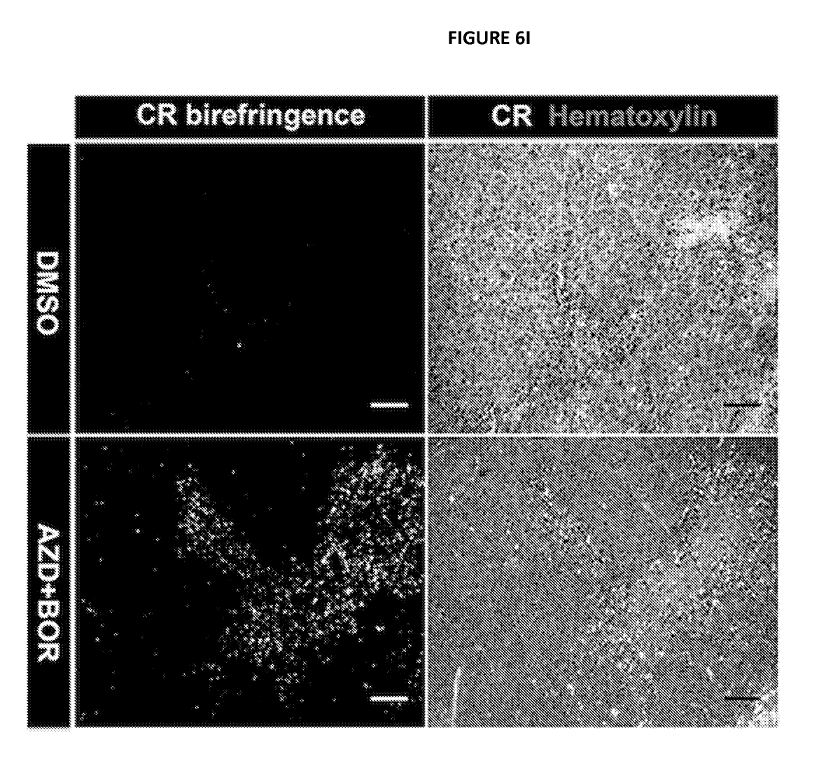

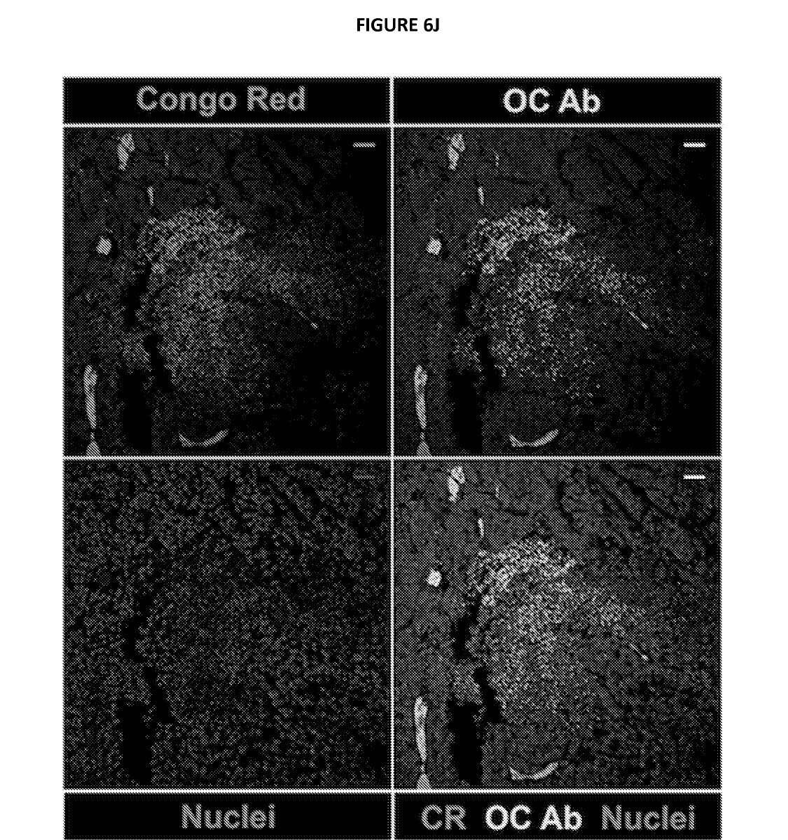

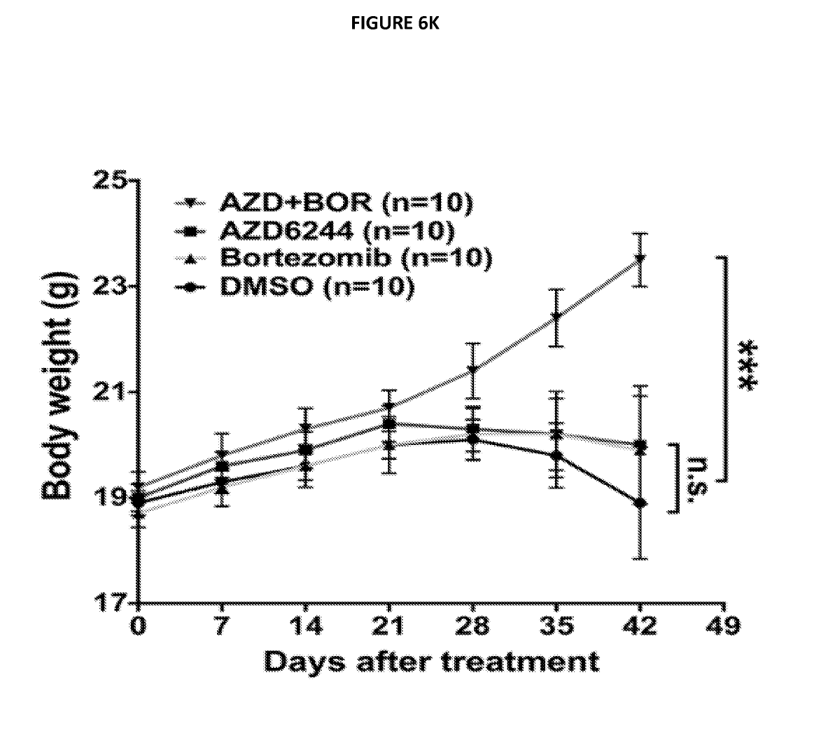

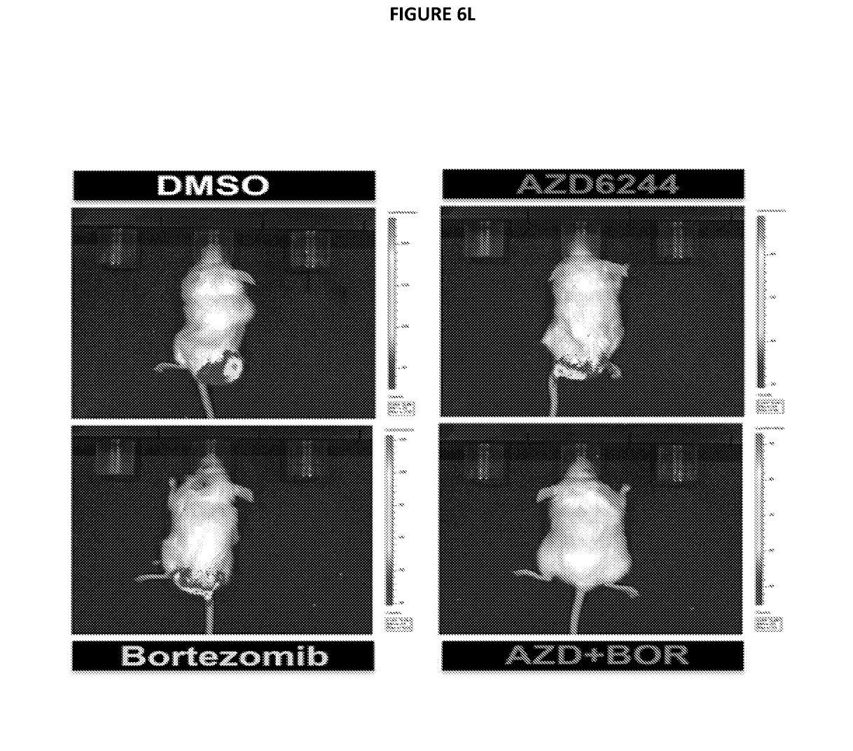

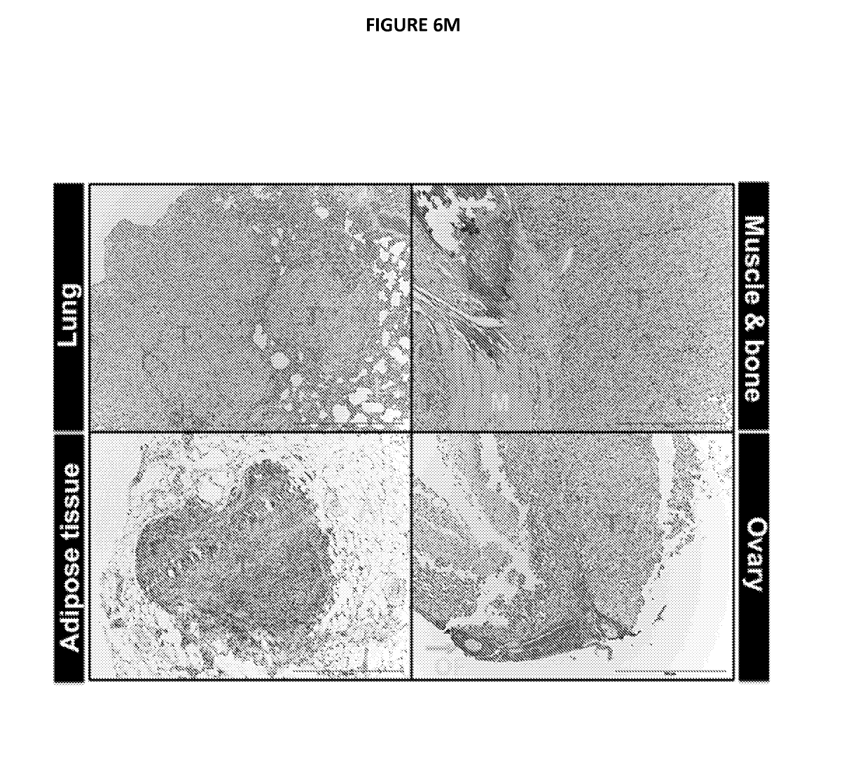

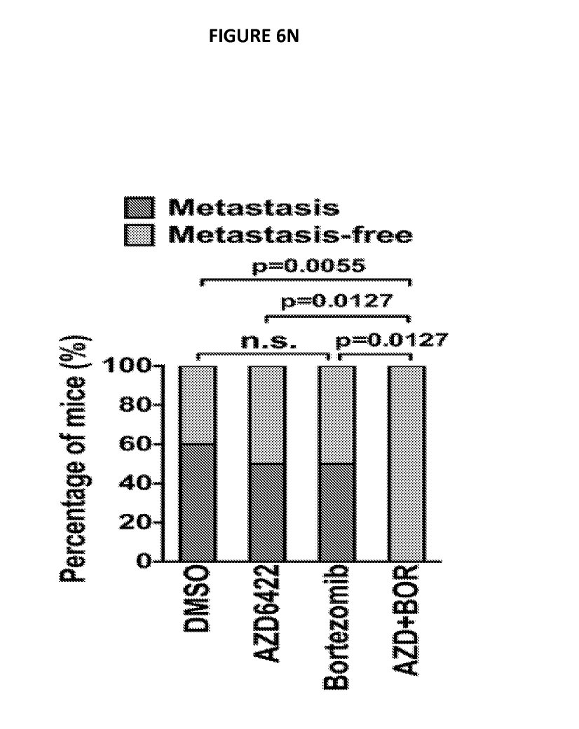

MEK and proteasome inhibition, individually, disturbed proteostasis in tumor cells to certain degrees; however, the combination of both augmented this effect and, accordingly, markedly impaired the growth and survival of human tumor cell lines (FIG. 6A). Of note, this combination did not impact primary cells (FIG. 6A).

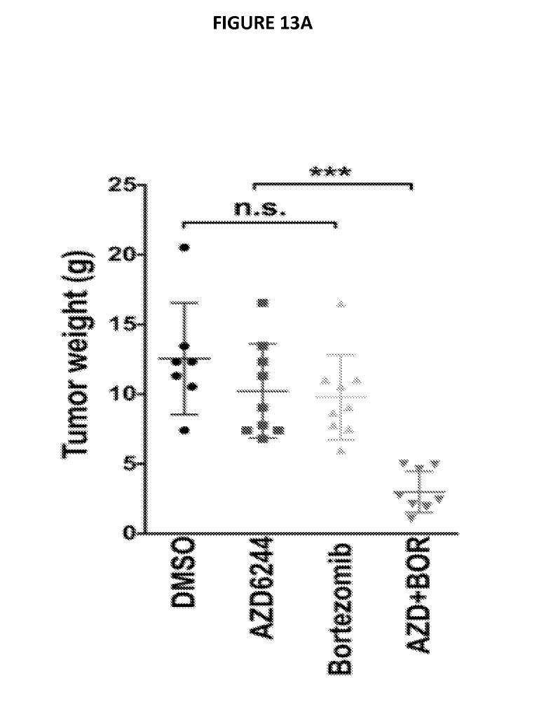

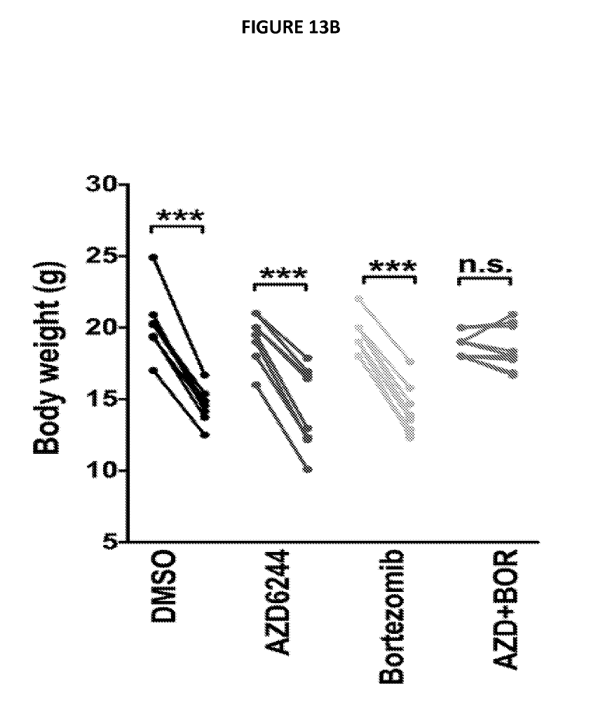

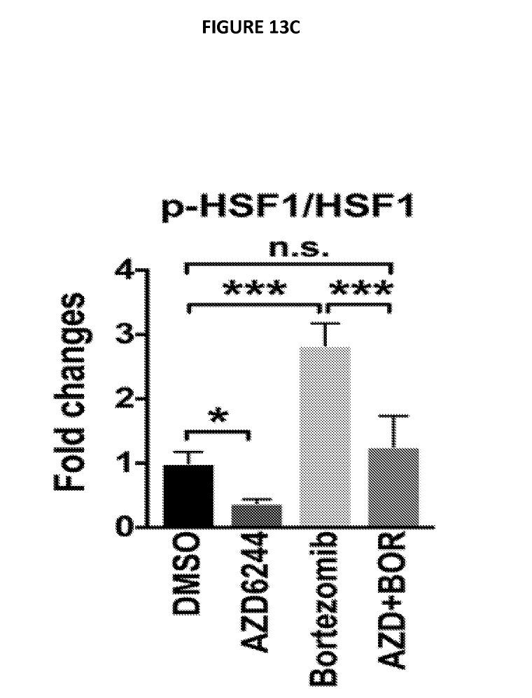

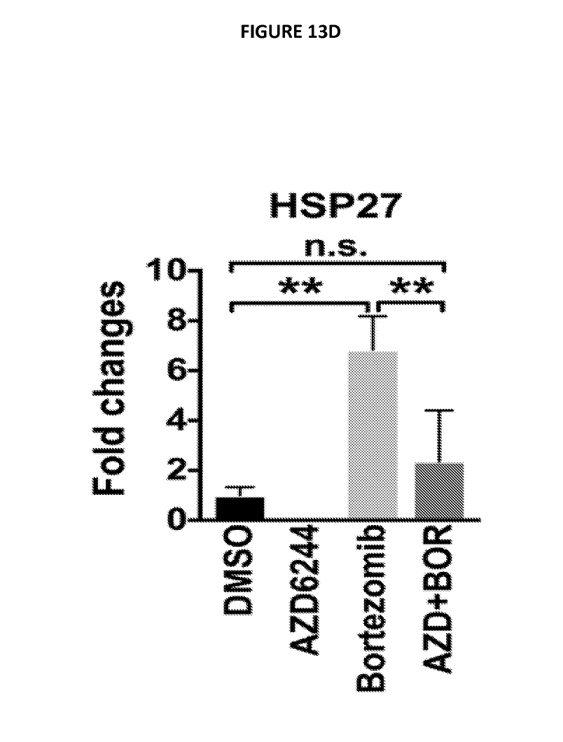

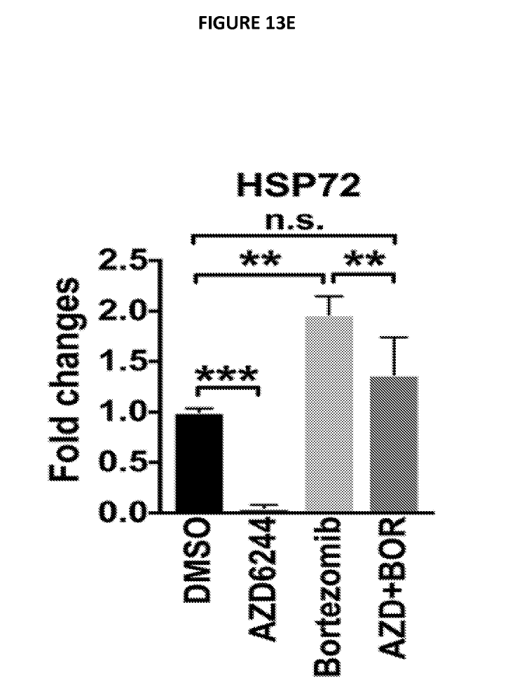

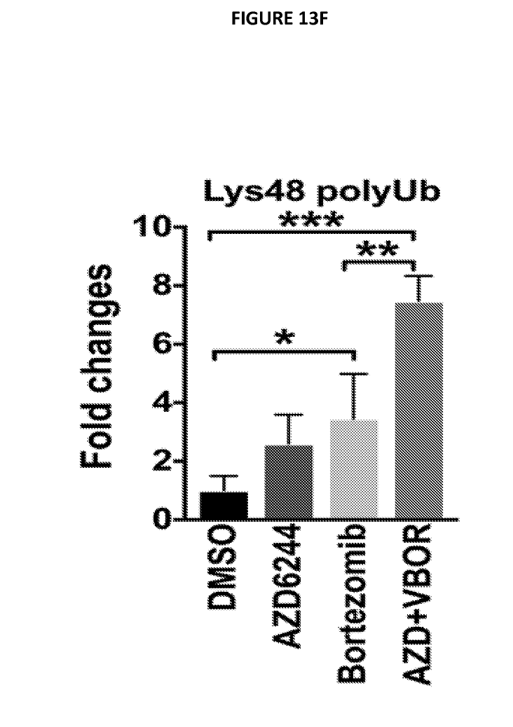

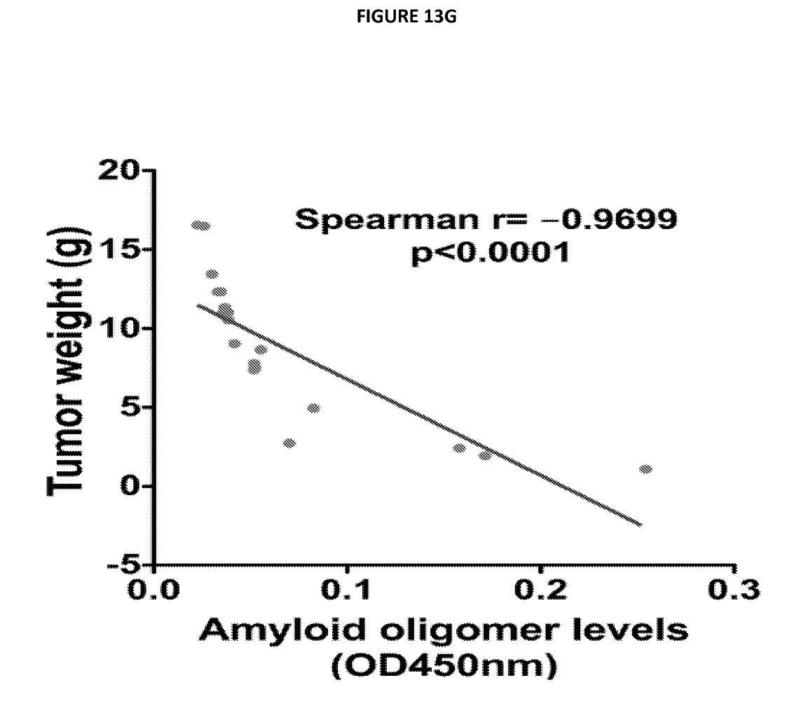

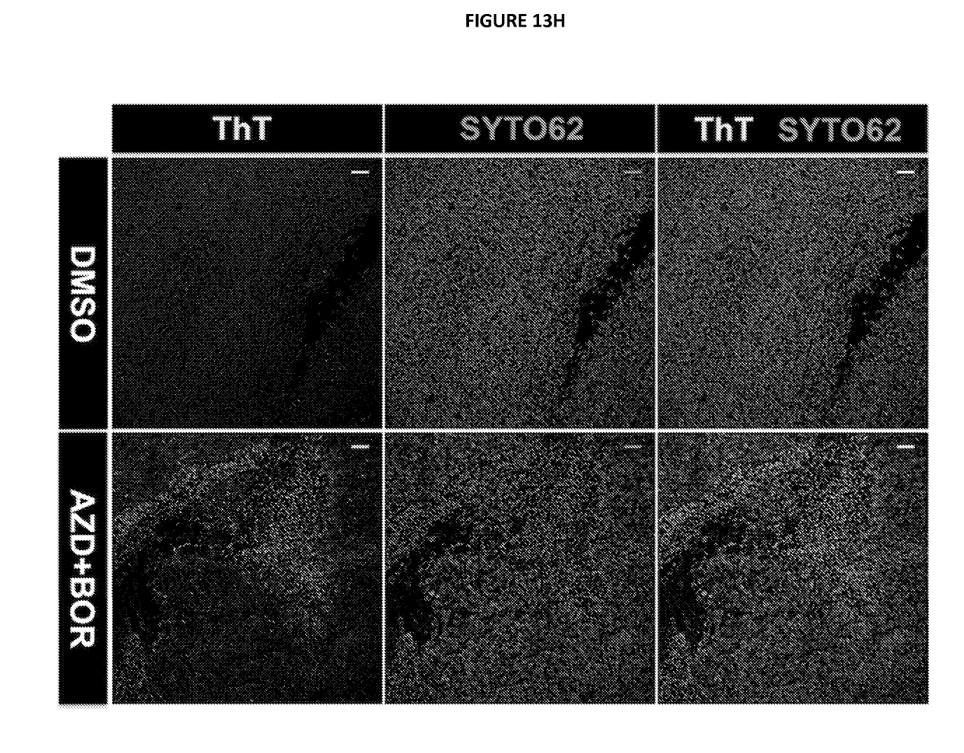

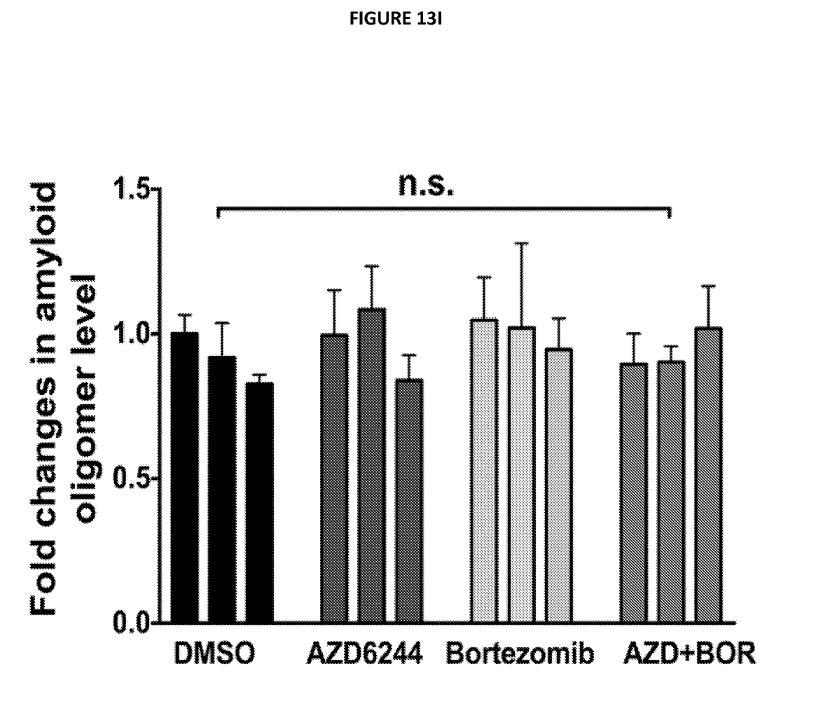

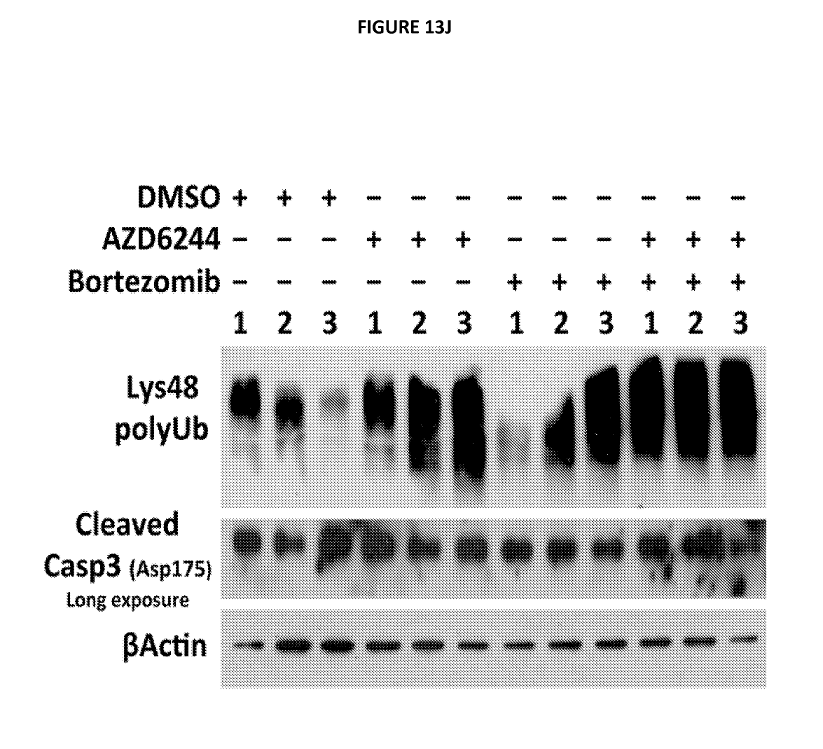

In vivo, whereas low doses of AZD6244 or Bortezomib alone exhibited no significant impacts on xenografted melanomas, the combination potently retarded their growth (FIGS. 6B and 13A). All mice receiving the combined treatment remained alive and their body weights remained constant; in contrast, all mice in the other groups died and lost about 25% of body weight (FIGS. 6C and 13B). AZD6244 or Bortezomib alone slightly elevated ubiquitination in tumors; however, the combination markedly aggravated this effect (FIGS. 6D and 13C-F). While Bortezomib induced HSF1 Ser326 phosphorylation and HSP expression, AZD6244 co-treatment suppressed this stress response and induced caspase 3 cleavage (FIGS. 6D and 13C-F). Accordingly, AOs were evidently elevated in tumors receiving combined treatment (FIG. 6E). Of particular interest is an inverse correlation between amounts of AOs and tumor masses (FIG. 13G), supporting an adverse impact of AOs on malignant growth. Congruent with amyloidogenesis, tumors receiving combined treatment displayed potent seeding capacities and enhancement of CR staining (FIGS. 6F and 6G-H). Intratumoral AFs were further demonstrated by the hallmark birefringence of CR staining (FIG. 6I), ThT labeling (FIG. 13H), and immunostaining with OC antibodies (FIG. 6J). In contrast, the combined treatment did not induce AOs and apoptosis in primary tissues of the same tumor-bearing mice, despite elevated ubiquitination (FIGS. 13I and 13J).

To investigate whether the combined treatment impedes experimental metastasis, we intravenously injected melanoma cells expressing a luciferase transgene into NOD/SCID mice. During a 6-week period, only mice receiving combined treatment gained body weight (FIG. 6K), suggesting improved health. In vivo imaging detected luminescence in 40% of mice treated with DMSO, AZD6244, or Bortezomib alone (FIG. 6L). Histological examination confirmed melanoma metastases to the lung, skeletal muscle, adipose tissue, and ovary (FIG. 6M and Table 2). In contrast, none of the mice receiving combined treatment displayed discernible luminescence or metastases (FIGS. 6L and 6N). Together, these results demonstrate that combined MEK and proteasome inhibition provokes proteotoxic stress and amyloidogenesis within tumors, and exerts robust anti-neoplastic effects.

TABLE 2
Summary of the melanoma metastasis study, Related to FIG. 6

| Mouse | | Lumines- | |
|---|---|---|---|
| ID# | Treatment | cence | Major histological findings |
| 1 | DMSO | positive | A large invasive melanoma invading |
| | | | muscle and adjacent to bone and |
| | | | cartilage |
| 2 | DMSO | negative | Medium-sized melanomas invading |
| | | | and partially replacing the lung |
| 3 | DMSO | negative | No evidence of neoplastic cells |
| 4 | DMSO | positive | A large melanoma, no defined tissue |
| | | | location |
| 5 | DMSO | positive | A large invasive melanoma invading |
| | | | muscle and pelvic adipose tissue |
| 6 | DMSO | positive | A large subcutaneous melanoma |
| 7 | DMSO | negative | No evidence of neoplastic cells |
| 8 | DMSO | negative | A large thymic lymphoma |
| 9 | DMSO | negative | No evidence of neoplastic cells |
| 10 | DMSO | negative | Small focus of melanoma invading |
| | | | the lung |
| 1 | AZD6244 | negative | No evidence of neoplastic cells |
| 2 | AZD6244 | negative | No evidence of neoplastic cells |
| 3 | AZD6244 | negative | No evidence of neoplastic cells |
| 4 | AZD6244 | positive | A large invasive melanoma invading |
| | | | skeletal muscle, adjacent to cartilage |
| 5 | AZD6244 | positive | A large subcutaneous melanoma |
| 6 | AZD6244 | negative | No evidence of neoplastic cells |
| 7 | AZD6244 | negative | No evidence of neoplastic cells |
| 8 | AZD6244 | positive | A large invasive melanoma invading |
| | | | muscle and adjacent to bone |
| 9 | AZD6244 | positive | A large melanoma, no defined tissue |
| | | | location |
| 10 | AZD6244 | negative | A large invasive melanoma invading |
| | | | and replacing most of the ovary |
| 1 | VELCADE | positive | A large invasive melanoma invading |
| | | | muscle and pelvic adipose tissue |
| 2 | VELCADE | negative | One medium-sized melanoma |
| | | | invading the lung |
| 3 | VELCADE | negative | No evidence of neoplastic cells |
| 4 | VELCADE | positive | A large subcutaneous melanoma |
| 5 | VELCADE | positive | A large invasive melanoma invading |
| | | | muscle and adjacent to bone |
| 6 | VELCADE | negative | No evidence of neoplastic cells |
| 7 | VELCADE | positive | A large invasive melanoma invading |
| | | | skeletal muscle |
| 8 | VELCADE | negative | No evidence of neoplastic cells |
| 9 | VELCADE | negative | No evidence of neoplastic cells |
| 10 | VELCADE | negative | No evidence of neoplastic cells |
| 1 | AZD + VEL | negative | No evidence of neoplastic cells |
| 2 | AZD + VEL | negative | No evidence of neoplastic cells |
| 3 | AZD + VEL | negative | No evidence of neoplastic cells |
| 4 | AZD + VEL | negative | No evidence of neoplastic cells |
| 5 | AZD + VEL | negative | No evidence of neoplastic cells |
| 6 | AZD + VEL | negative | No evidence of neoplastic cells |
| 7 | AZD + VEL | negative | No evidence of neoplastic cells |
| 8 | AZD + VEL | negative | No evidence of neoplastic cells |
| 9 | AZD + VEL | negative | No evidence of neoplastic cells |
| 10 | AZD + VEL | negative | No evidence of neoplastic cells |

### Example 9. Amyloidogenesis is Tumor-Suppressive

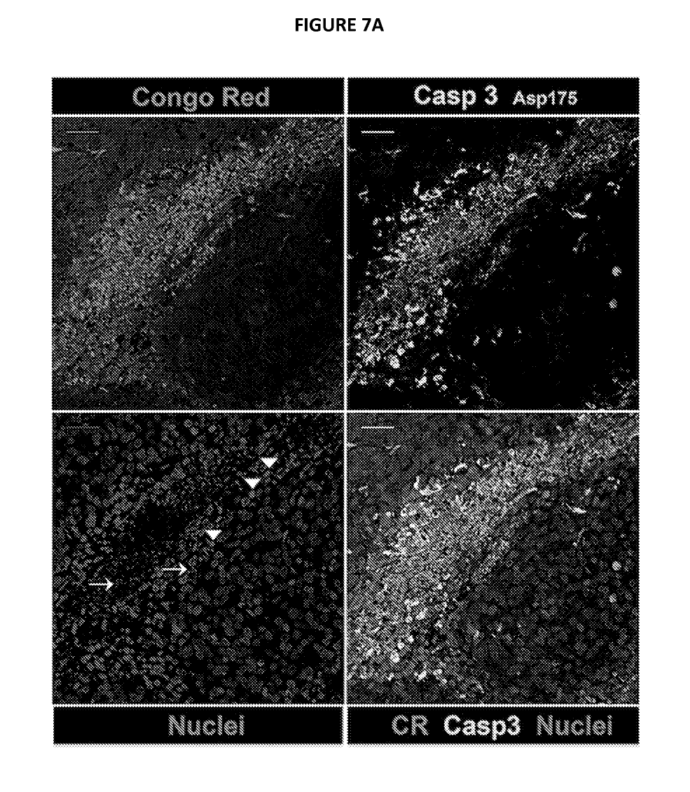

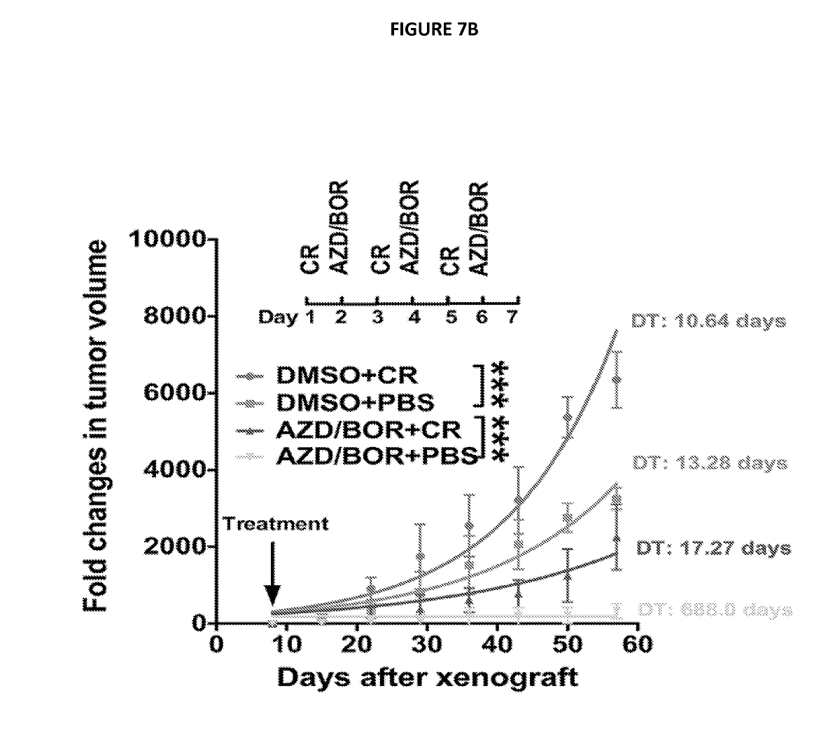

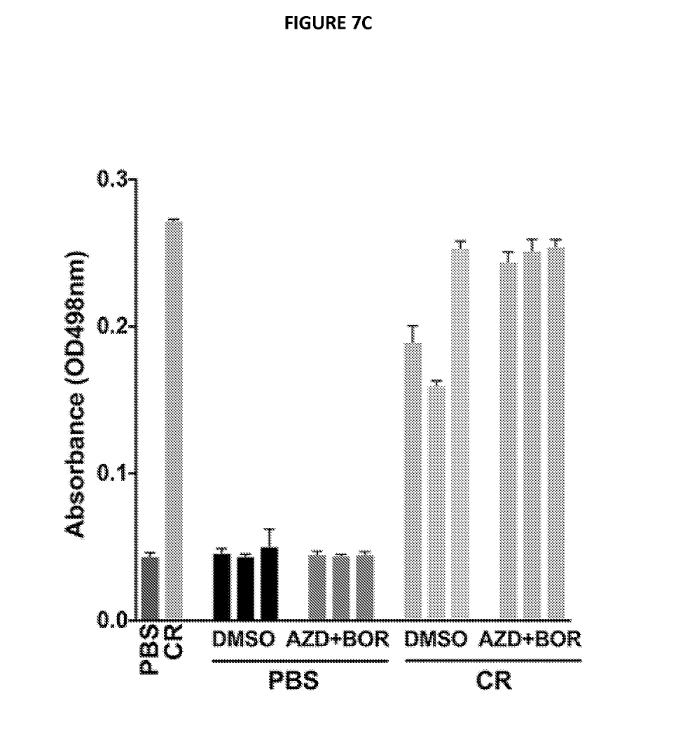

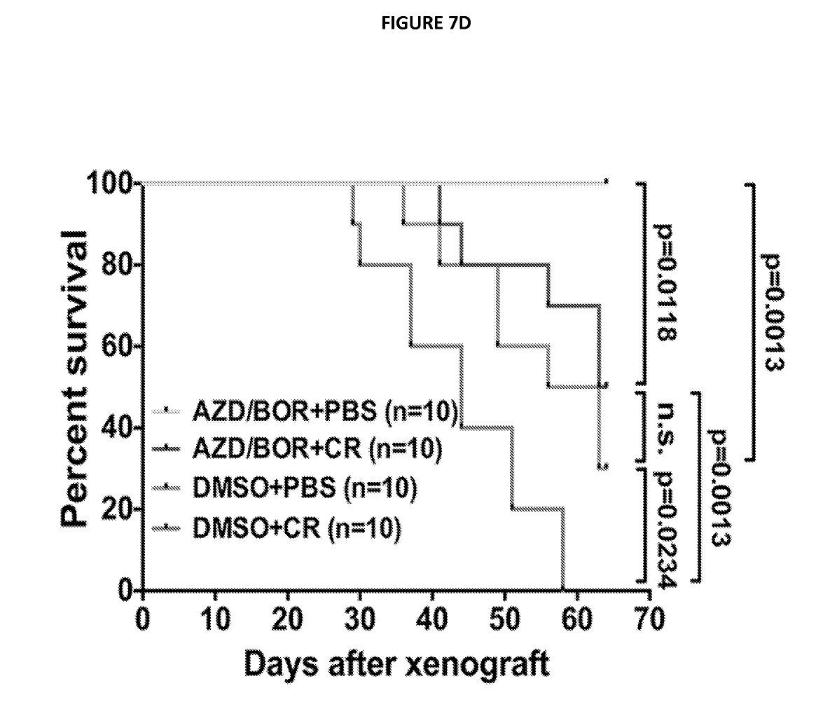

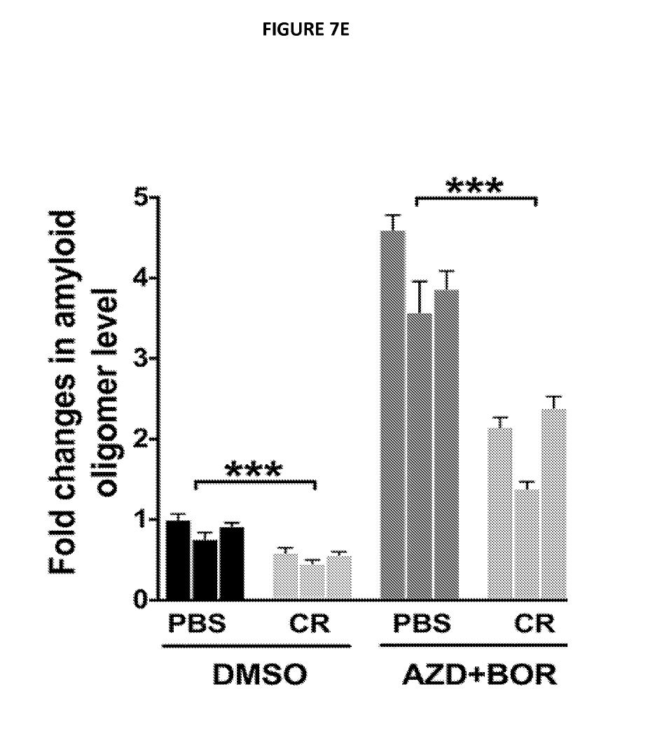

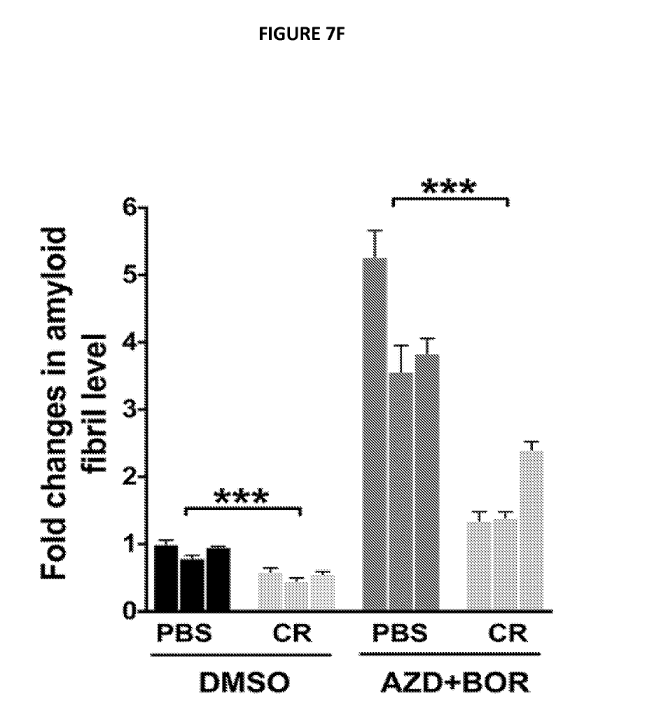

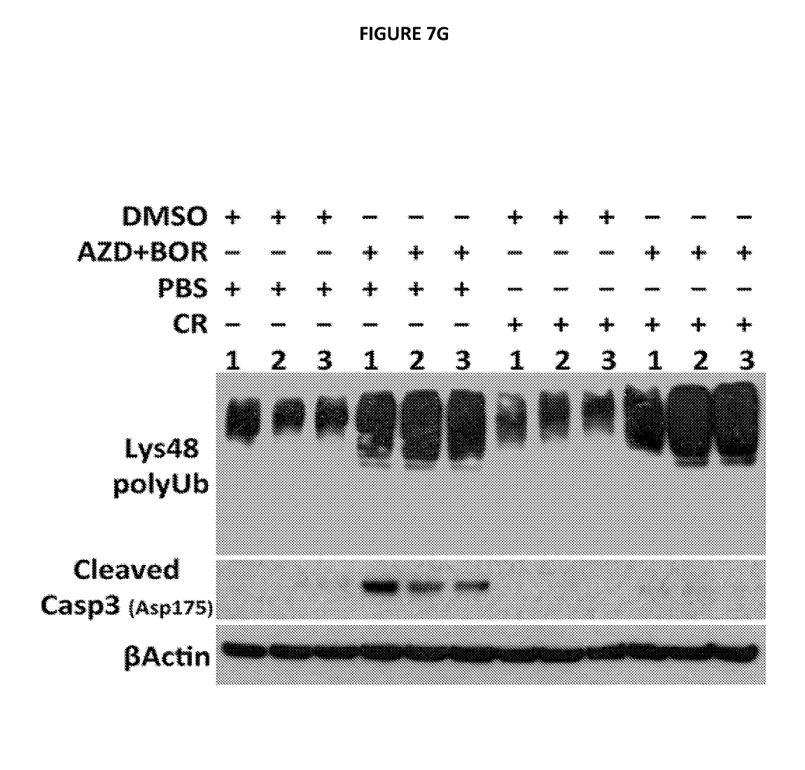

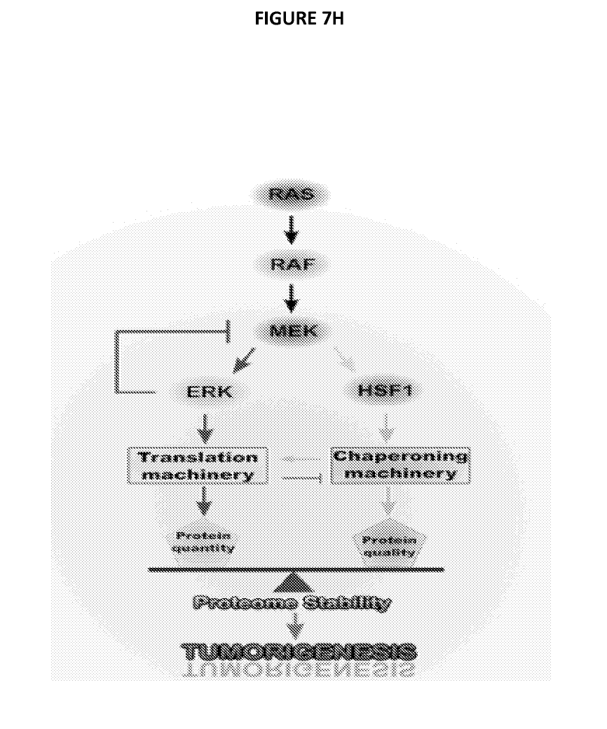

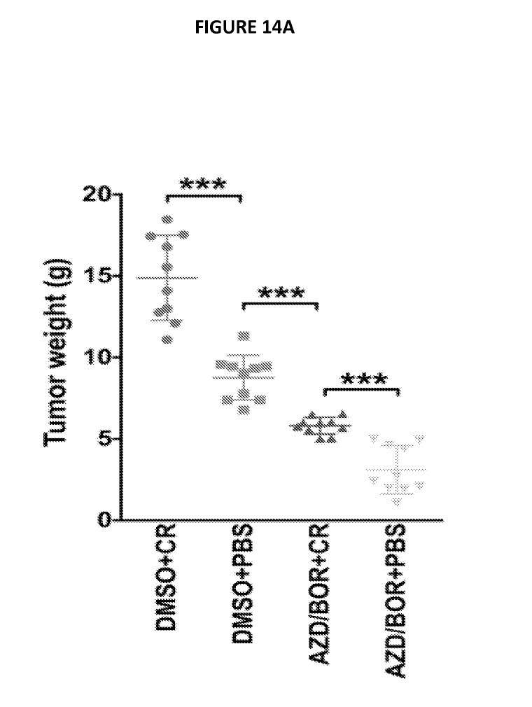

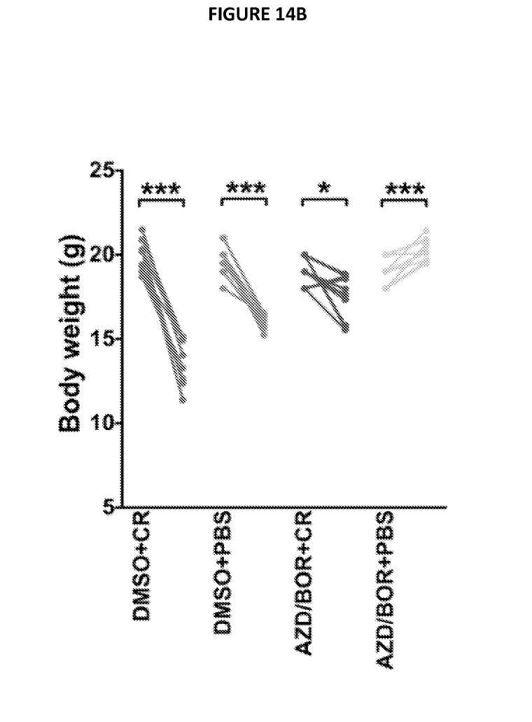

Evident apoptosis in tumor regions showing intense CR staining suggests a causative role of amyloidogenesis in treatment-induced toxicity (FIG. 7A). To confirm this, we blocked intratumoral amyloid induction via in vivo CR administration. CR not only accelerated melanoma growth but also potently antagonized the tumor suppression imposed by combined MEK and proteasome inhibition (FIG. 7B). Penetration of CR into tumor tissues was indicated by intense light absorption of tumor lysates at 498 nm (FIG. 7C), a characteristic of this amyloid stain (Sladewski et al., 2006). Congruent with enhanced malignancy, CR treatment enlarged tumor masses, deteriorated body conditions, and shortened animal survival (FIGS. 14A, 14B, and 7D).

While CR reduced amyloids in tumor tissues, it did not diminish ubiquitination (FIGS. 7E, 7F, and 7G). These results indicate no interference of CR with MEK and proteasome inhibitors, and further support a specific action of CR in blocking amyloid genesis. In accordance with accelerated growth, CR-treated tumors displayed reduced caspase 3 cleavage (FIG. 7G). Collectively, these results strongly suggest that amyloidogenesis is tumor-suppressive and evidently contributes to the anti-neoplastic effects of combined MEK and proteasome inhibition.

### Example 10. Discussion

A. HSF1 is a New MEK Substrate

Unexpectedly, our results reveal HSF1 as a physiological substrate for MEK, challenging the prevailing paradigm wherein ERK exclusively instigates the effects of RAS-RAF-MEK signaling. Our results further show that MEK activates but ERK inactivates HSF1. Importantly, our findings integrate these two seemingly contradictory actions and support the assembly of a ternary ERK-MEK-HSF1 protein complex. In aggregate, our findings propose a bifurcated, rather than a linear, RAS-RAF-MEK cascade. MEK, as a central nexus, both conveys upstream stimuli and governs two discrete but interconnected downstream effector pathways, of which one is mediated by ERK and the other by HSF1 (FIG. 7H). In a negative feedback fashion, ERK finely attunes HSF1 activation via inhibitory phosphorylation of MEK (FIG. 7H). While our studies focused on MEK-mediated Ser326 phosphorylation, other kinases can also regulate HSF1.

B. Guarding of Proteostasis by RAS-RAF-MEK Signaling

Our findings uncover a new function of RAS-RAF-MEK signaling in regulating proteostasis. Diverse proteotoxic stressors commonly activate MEK (FIG. 1A). Through HSF1 activation, RAS-RAF-MEK signaling heightens cellular chaperoning capacity to guard proteomic integrity.

MEK-HSF1 regulation could have key physiological implications. Mitogens stimulate RAS/MAPK signaling and downstream mTORC1 (Laplante and Sabatini, 2012). However, heightened protein synthesis driven by mTORC1 encumbers cellular protein quality-control machinery. It thus appears necessary for mitogens, via MEK, to concurrently mobilize the HSF1-controlled chaperone system to ensure productive protein synthesis and, thereby, avert proteomic imbalance. Interestingly, MEK also governs translation capacity via HSF1 (FIG. 4K). Thus, RAS-RAF-MEK signaling synchronizes protein quantity- and quality-control machineries to support cellular growth.

It is also tempting to speculate that RAS-RAF-MEK signaling may antagonize protein-misfolding diseases, such as amyloidosis, via guarding proteostasis.

C. Proteomic Instability of Cancer

Our findings pinpoint a pro-amyloidogenic nature of malignant state. The susceptibility of malignant cells to amyloid genesis likely originates from their debilitated proteostatic state, which is particularly vulnerable to perturbations. Unlike non-transformed cells, malignant cells constantly endure proteomic imbalance, evidenced by elevated basal levels of amyloids (FIGS. 5X and 5Y). Accordingly, HSF1, otherwise latent in primary cells, is constitutively mobilized in tumor cells to retain the fragile proteomic equilibrium (FIG. 7H). Proteomic chaos inevitably ensues following either HSF1 inactivation or even mild proteotoxic insults. Hence, tumors cells rely on HSF1 to sustain their malignant phenotypes (Dai et al., 2007). In contrast, the lack of intrinsic proteotoxic stress empowers primary cells to effectively buffer intense proteomic fluctuations and thereby avert deleterious consequences—aggregation and amyloidogenesis.

Excitingly, the distinct susceptibilities to proteomic perturbation between primary and malignant cells may be exploited to combat malignancy. Our findings support important roles for proteotoxic stress and amyloidogenesis in the toxicity of MEK inhibition in malignancy. Through protein destabilization, MEK inhibitors act as a proteotoxic stressor, mechanistically distinct from proteasome inhibitors. When applied as single agent, a MEK or proteasome inhibitor is incompetent to distress tumor proteostasis. However, combinatorial application exerts a profound impact, eliciting amyloidogenesis. Importantly, our findings strongly suggest a tumor-suppressive nature of amyloidogenesis (FIGS. 5O-5R and 7B). These findings imply that amyloidogenesis, indicative of grave proteomic imbalance, may be of prognostic value in monitoring tumor progression and evaluating therapeutic responses. Conceptually, our findings suggest that proteomic instability is an intrinsic characteristic associated with malignant state and that, therefore, disruption of fragile tumor proteostasis may be a feasible therapeutic strategy.

### Example 11. A Method of Treatment

A patient having metastatic melanoma is treated with a combination of a MEK inhibitor and a proteasome inhibitor. Namely, the patient is treated with selumetinib as the MEK inhibitor and bortezomib as the proteasome inhibitor. The selumetinib is administered at 0.32 mg/Kg after reconstitution of a dry powder with water for daily intravenous injection or 0.64 mg/Kg with food through daily oral administration. The bortezomib is administered at 0.04 mg/Kg after reconstitution of a dry power with water for daily intravenous injection.

The patient is expected to show a reduction in the growth of the tumor, the size of the tumor, or other clinical signs and symptoms of melanoma.

### REFERENCES

Tang, Z., Dai, S., He, Y., Doty, R., Shultz, L. D., Sampson, S. B., and Dai, C. (2015). MEK Guards Proteome Stability and Inhibits Tumor-Suppressive Amyloidogenesis via HSF1. Cell 160, 729-744, which is incorporated by reference herein.
Ahn, N. G., Seger, R., Bratlien, R. L., Diltz, C. D., Tonks, N. K., and Krebs, E. G. (1991). Multiple components in an epidermal growth factor-stimulated protein kinase cascade. In vitro activation of a myelin basic protein/microtubule-associated protein 2 kinase J Biol. Chem. 266, 4220-4227.
Alavez, S., Vantipalli, M. C., Zucker, D. J., Kiang, I. M., and Lithgow, G. J. (2011). Amyloid-binding compounds maintain protein homeostasis during ageing and extend lifespan. Nature 472, 226-229.

Balch, W. E., Morimoto, R. I., Dillin, A., and Kelly, J. W. (2008). Adapting proteostasis for disease intervention. Science 319, 916-919.
Brunet, A., Pages, G., and Pouyssegur, J. (1994). Constitutively active mutants of MAP kinase kinase (MEK1) induce growth factor-relaxation and oncogenicity when expressed in fibroblasts. Oncogene 9, 3379-3387.
Brunet, A., Pages, G., and Pouyssegur, J. (1994). Growth factor-stimulated MAP kinase induces rapid retrophosphorylation and inhibition of MAP kinase kinase (MEK1). FEBS Lett. 346, 299-303.
Catalanotti, F., Reyes, G., Jesenberger, V., Galabova-Kovacs, G., de Matos Simoes, R., Carugo, O., and Baccarini, M. (2009). A Mek1-Mek2 heterodimer determines the strength and duration of the Erk signal. Nat. Struct. Mol. Biol. 16, 294-303.
Chiti, F., and Dobson, C. M. (2006). Protein misfolding, functional amyloid, and human disease. Annu. Rev. Biochem. 75, 333-366.
Chu, B., Zhong, R., Soncin, F., Stevenson, M. A., and Calderwood, S. K. (1998). Transcriptional activity of heat shock factor 1 at 37 degrees C. is repressed through phosphorylation on two distinct serine residues by glycogen synthase kinase 3 and protein kinases Calpha and Czeta. J. Biol. Chem. 273, 18640-18646.
Clausson, C. M., Allalou, A., Weibrecht, I., Mahmoudi, S., Farnebo, M., Landegren, U., Wahlby, C., and Soderberg, O. (2011). Increasing the dynamic range of in situ PLA. Nat. Methods 8, 892-893.
Dai, C., Dai, S., and Cao, J. (2012). Proteotoxic stress of cancer: implication of the heat-shock response in oncogenesis. J. Cell. Physiol. 227, 2982-2987.
Dai, C., Santagata, S., Tang, Z., Shi, J., Cao, J., Kwon, H., Bronson, R. T., Whitesell, L., and Lindquist, S. (2012). Loss of tumor suppressor NF1 activates HSF1 to promote carcinogenesis. J. Clin. Invest. 122, 3742-3754.
Dai, C., Whitesell, L., Rogers, A. B., and Lindquist, S. (2007). Heat shock factor 1 is a powerful multifaceted modifier of carcinogenesis. Cell 130, 1005-1018.
Dhillon, A. S., Hagan, S., Rath, O., and Kolch, W. (2007). MAP kinase signalling pathways in cancer. Oncogene 26, 3279-3290.
Eisenberg, D., and Jucker, M. (2012). The amyloid state of proteins in human diseases. Cell 148, 1188-1203.
Favata, M. F., Horiuchi, K. Y., Manos, E. J., Daulerio, A. J., Stradley, D. A., Feeser, W. S., Van Dyk, D. E., Pitts, W. J., Earl, R. A., Hobbs, F., et al. (1998). Identification of a novel inhibitor of mitogen-activated protein kinase kinase J Biol. Chem. 273, 18623-18632.
Glabe, C. G. (2008). Structural classification of toxic amyloid oligomers. J. Biol. Chem. 283, 29639-29643.
Guettouche, T., Boellmann, F., Lane, W. S., and Voellmy, R. (2005). Analysis of phosphorylation of human heat shock factor 1 in cells experiencing a stress. BMC Biochem. 6, 4.
Hsu, A. L., Murphy, C. T., and Kenyon, C. (2003). Regulation of aging and age-related disease by DAF-16 and heat-shock factor. Science 300, 1142-1145.
Isaacs, J. S., Jung, Y. J., Mimnaugh, E. G., Martinez, A., Cuttitta, F., and Neckers, L. M. (2002). Hsp90 regulates a von Hippel Lindau-independent hypoxia-inducible factor-1 alpha-degradative pathway. J. Biol. Chem. 277, 29936-29944.
Jin, X., Moskophidis, D., and Mivechi, N. F. (2011). Heat shock transcription factor 1 is a key determinant of HCC development by regulating hepatic steatosis and metabolic syndrome. Cell Metab. 14, 91-103.
Kawaguchi, Y., Kovacs, J. J., McLaurin, A., Vance, J. M., Ito, A., and Yao, T. P. (2003). The deacetylase HDAC6 regulates aggresome formation and cell viability in response to misfolded protein stress. Cell 115, 727-738.
Kayed, R., Head, E., Sarsoza, F., Saing, T., Cotman, C. W., Necula, M., Margol, L., Wu, J., Breydo, L., Thompson, J. L., et al. (2007). Fibril specific, conformation dependent antibodies recognize a generic epitope common to amyloid fibrils and fibrilar oligomers that is absent in prefibrillar oligomers. Mol. Neurodegener. 2, 18.
Kayed, R., Head, E., Thompson, J. L., McIntire, T. M., Milton, S. C., Cotman, C. W., and Glabe, C. G. (2003). Common structure of soluble amyloid oligomers implies common mechanism of pathogenesis. Science 300, 486-489.
Kim, W., Bennett, E. J., Huttlin, E. L., Guo, A., Li, J., Possemato, A., Sowa, M. E., Rad, R., Rush, J., Comb, M. J., et al. (2011). Systematic and quantitative assessment of the ubiquitin-modified proteome. Mol. Cell 44, 325-340.
Laplante, M., and Sabatini, D. M. (2012). mTOR signaling in growth control and disease. Cell 149, 274-293.
Lindquist, S. (1986). The heat-shock response. Annu. Rev. Biochem. 55, 1151-1191.
Massey, A. J., Williamson, D. S., Browne, H., Murray, J. B., Dokurno, P., Shaw, T., Macias, A. T., Daniels, Z., Geoffroy, S., Dopson, M., et al. (2010). A novel, small molecule inhibitor of Hsc70/Hsp70 potentiates Hsp90 inhibitor induced apoptosis in HCT116 colon carcinoma cells. Cancer Chemother. Pharmacol. 66, 535-545.
Meng, L., Gabai, V. L., and Sherman, M. Y. (2010). Heat-shock transcription factor HSF1 has a critical role in human epidermal growth factor receptor-2-induced cellular transformation and tumorigenesis. Oncogene 29, 5204-5213.
Min, J. N., Huang, L., Zimonjic, D. B., Moskophidis, D., and Mivechi, N. F. (2007). Selective suppression of lymphomas by functional loss of Hsf1 in a p53-deficient mouse model for spontaneous tumors. Oncogene 26, 5086-5097.
Morimoto, R. I. (2008). Proteotoxic stress and inducible chaperone networks in neurodegenerative disease and aging. Genes Dev. 22, 1427-1438.
Morris, E. J., Jha, S., Restaino, C. R., Dayananth, P., Zhu, H., Cooper, A., Carr, D., Deng, Y., Jin, W., Black, S., et al. (2013). Discovery of a novel ERK inhibitor with activity in models of acquired resistance to BRAF and MEK inhibitors. Cancer Discov. 3, 742-750.
Muller, L., Schaupp, A., Walerych, D., Wegele, H., and Buchner, J. (2004). Hsp90 regulates the activity of wild type p53 under physiological and elevated temperatures. J. Biol. Chem. 279, 48846-48854.
Neckers, L., and Workman, P. (2012). Hsp90 molecular chaperone inhibitors: are we there yet? Clin. Cancer Res. 18, 64-76.
Nikolaev, S. I., Rimoldi, D., Iseli, C., Valsesia, A., Robyr, D., Gehrig, C., Harshman, K., Guipponi, M., Bukach, O., Zoete, V., et al. (2012). Exome sequencing identifies recurrent somatic MAP2K1 and MAP2K2 mutations in melanoma. Nat. Genet. 44, 133-139.
Oerlemans, R., Franke, N. E., Assaraf, Y. G., Cloos, J., van Zantwijk, I., Berkers, C. R., Scheffer, G. L., Debipersad, K., Vojtekova, K., Lemos, C., et al. (2008). Molecular basis of bortezomib resistance: proteasome subunit beta5 (PSMB5) gene mutation and overexpression of PSMB5 protein. Blood 112, 2489-2499.
Ohori, M., Kinoshita, T., Okubo, M., Sato, K., Yamazaki, A., Arakawa, H., Nishimura, S., Inamura, N., Nakajima, H., Neya, M., et al. (2005). Identification of a selective ERK inhibitor and structural determination of the inhibitor-ERK2 complex. Biochem. Biophys. Res. Commun. 336, 357-363.
Orr, H. T., and Zoghbi, H. Y. (2007). Trinucleotide repeat disorders. Annu Rev Neurosci 30, 575-621.

Pickart, C. M., and Eddins, M. J. (2004). Ubiquitin: structures, functions, mechanisms. Biochim Biophys. Acta 1695, 55-72.
Roux, P. P., and Blenis, J. (2004). ERK and p38 MAPK-activated protein kinases: a family of protein kinases with diverse biological functions. Microbiol. Mol. Biol. Rev. 68, 320-344.
Sanchez, I., Mahlke, C., and Yuan, J. (2003). Pivotal role of oligomerization in expanded polyglutamine neurodegenerative disorders. Nature 421, 373-379.
Sladewski, T. E., Shafer, A. M., and Hoag, C. M. (2006). The effect of ionic strength on the UV-vis spectrum of congo red in aqueous solution. Spectrochim Acta A. Mol. Biomol. Spectrosc. 65, 985-987.
Taipale, M., Jarosz, D. F., and Lindquist, S. (2010). HSP90 at the hub of protein homeostasis: emerging mechanistic insights. Nat. Rev. Mol. Cell Biol. 11, 515-528.
Xiao, X., Zuo, X., Davis, A. A., McMillan, D. R., Curry, B. B., Richardson, J. A., and Benjamin, I. J. (1999). HSF1 is required for extra-embryonic development, postnatal growth and protection during inflammatory responses in mice. EMBO J. 18, 5943-5952.
Yeh, T. C., Marsh, V., Bernat, B. A., Ballard, J., Colwell, H., Evans, R. J., Parry, J., Smith, D., Brandhuber, B. J., Gross, S., et al. (2007). Biological characterization of ARRY-142886 (AZD6244), a potent, highly selective mitogen-activated protein kinase kinase 1/2 inhibitor. Clin. Cancer Res. 13, 1576-1583.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A method of treating cancer comprising administering a MEK inhibitor in combination with a proteasome inhibitor.

2. The method of claim 1, wherein the cancer is a solid tumor.

3. The method of claim 2, wherein the solid tumor is biliary (cholangiocarcinoma), bladder cancer, brain cancer, breast cancer, cervical cancer, colorectal cancer, endometrial cancer, epidermoid carcinoma, esophageal carcinoma, gallbladder cancer, gastric (stomach) cancer, glioblastoma, glioma, head and neck cancers, hepatocellular (liver) carcinoma, kidney cancer, lung cancer, mesothelioma, non-small cell lung cancer, ovarian, pancreatic cancer, pediatric malignancies, prostate cancer, renal cancer, sarcomas, skin cancer (including melanoma), small bowel adenocarcinoma, small cell lung cancer, testicular cancer, or thyroid cancer.

4. The method of claim 2, wherein the solid tumor is melanoma.

5. The method of claim 1, wherein the cancer has at least one mutation chosen from a NF1, RAS (including N-, K-, and H-RAS), RAF (including A-, B-, and C-RAF), and MEK (including MEK1 and MEK2) mutation.

6. The method of claim 5, wherein the cancer has at least a RAS mutation.

7. The method of claim 6, wherein the RAS mutation is in at least codon 12, 13, or 61.

8. The method of claim 5, wherein the cancer has at least a RAF mutation.

9. The method of claim 8, wherein the RAF mutation is in at least codon 600.

10. The method of claim 5, wherein the cancer has at least a MEK1 or MEK2 mutation.

11. The method of claim 10, wherein the MEK1 mutation is at least P124S or S203K, or wherein the MEK2 mutation is at least Q60P.

12. (canceled)

13. The method of claim 1, wherein the MEK inhibitor is selumetinib (AZD6244), trametinib (GSK1120212), binimetinib (MEK162), PD-325901, cobimetinib, PD184352 (CI-1040), U0126-EtOH, refametinib (RDEA119), PD98059, BIX 02189, pimasertib (AS-703026), SL-327, BIX 02188, AZD8330, TAK-733, honokiol, or PD318088, PD0325901, WX-554, GDC-0623, E6201, RO4987655, RO5126766.

14. The method of claim 13, wherein the MEK inhibitor is selumetinib.

15. The method of claim 1, wherein the proteasome inhibitor is bortezomib, lactacystin, disulfiram, epigallocatcechin-3-gallate, salinosporamide A, carfilzomib, oprozomib (ONX 0912), delanzomib (CEP-18770), MLN9708, epoxomicin, MG132, ixazomib (MLN2238), PI-1840, or celastrol.

16. The method of claim 15, wherein the proteasome inhibitor is bortezomib.

17. The method of claim 1, wherein the proteasome inhibitor and the MEK inhibitor are administered at a dosage that does not create a therapeutic benefit when either agent is administered alone.

18. The method of claim 1, wherein selumetinib is administered at about 5 mg/Kg and bortezomib is administered at about 0.5 mg/Kg.

19. The method of claim 1, wherein the cancer is resistant to treatment with at least one of a proteasome inhibitor or a MEK inhibitor.

20. The method of claim 1, wherein the combination therapy produces a synergistic effect.

21. The method of claim 1, wherein the cancer is resistant to treatment with at least one of a proteasome inhibitor or a MEK inhibitor.
